(19) United States
(12) Patent Application Publication
(10) Pub. No.: US 2025/0115545 A1
(43) Pub. Date: Apr. 10, 2025

(54) TERPENE-CONTAINING LIPIDOIDS FOR GENE DELIVERY

(71) Applicant: University of San Diego, San Diego, CA (US)

(72) Inventors: Peter M. Iovine, San Diego, CA (US); Gopi Nath Vemuri, San Diego, CA (US)

(21) Appl. No.: 18/730,688

(22) PCT Filed: Jan. 20, 2023
(86) PCT No.: PCT/US2023/011251

§ 371 Date: Jul. 19, 2024

Related U.S. Application Data
(60) Provisional application No. 63/301,801, filed on Jan. 21, 2022.
(60) Provisional application No. 63/409,686, filed on Sep. 23, 2022.

(51) Int. Cl. C07C 229/34; C07C 219/12; C07D 211/26; C07D 241/04

(52) U.S. Cl. CPC C07C 229/34; C07C 219/12; C07D 211/26; C07D 241/04; C07C 2601/14; C07C 2601/16

(57) ABSTRACT

Terpene-containing lipidoid compounds, methods of preparing such compositions, and the use of these, compositions in gene delivery applications are disclosed.

### FIELD OF THE INVENTION

The present invention relates generally to novel terpene-derived compounds, compositions containing such compounds, methods of preparing these compounds, and the use of these compositions in gene delivery.

### BACKGROUND OF THE INVENTION

Efficient and safe delivery is one of the most significant challenges in the field of biological therapeutics. In the delivery of mRNA, for example, efficient transfection involves not only target specificity but also cell entry, endosomal escape, and translation. Lipid nanoparticles (LNPs) have proven effective for the delivery of biological therapeutics but especially nucleic acid-based therapies. A typical LNP contains a structural lipid, a PEG-containing lipid, and a phospholipid (often unsaturated). In addition, a pH sensitive (ionizable) lipid is included and serves a functional role in the delivery of the cargo. Typically, pH-sensitive lipids contain one or more amine functionalities that can be protonated (cationic) under certain physiological conditions. It is widely reported that the protonation of the amine-containing lipid facilitates endosomal escape thereby releasing the therapeutic agent to the interior of the cell.

Although a large number of cationic lipids have been discovered through high throughput screens, there remains a significant need for novel structures with strong safety profiles, efficient delivery characteristics, and high target specificity. Studies that correlate cationic lipid structure with desirable delivery characteristics are lacking. Lipid tail branching and unsaturation are two structural parameters that impact delivery efficiency

Terpenes are an abundant class of naturally occurring unsaturated hydrocarbon. The use of terpenes, chemically modified terpenes, or terpene-containing structures in the field of gene delivery is extremely limited. Given their abundance and structural diversity, terpenes represent an attractive feedstock for the construction of gene delivery agents.

The compositions and methods of the present disclosure have wide applicability to a diverse number of fields, including gene therapy and the production of cell-based therapeutics.

### SUMMARY OF THE INVENTION

In one aspect, the present composition can be a terpene-derived cationic lipidoid compound represented by Formula (I), Formula (II) or Formula (III):

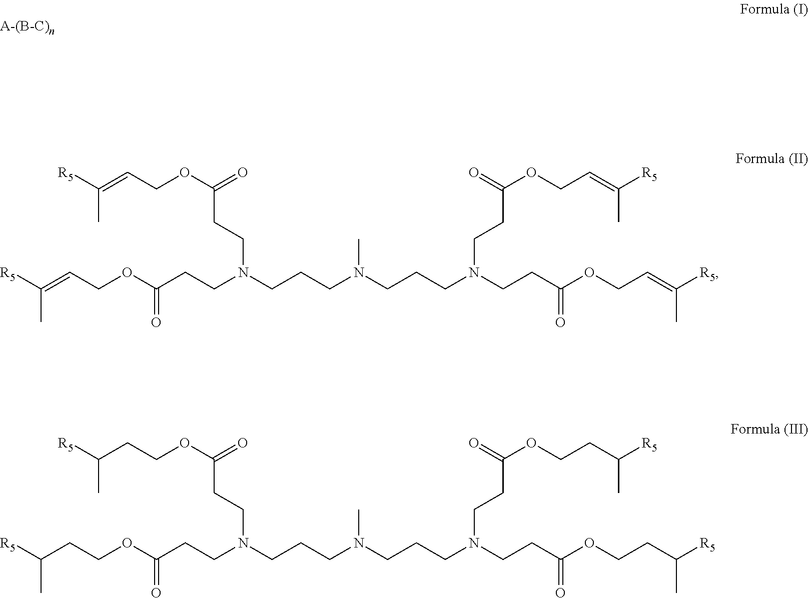

wherein:

A is A′ or A″,

A′ is:

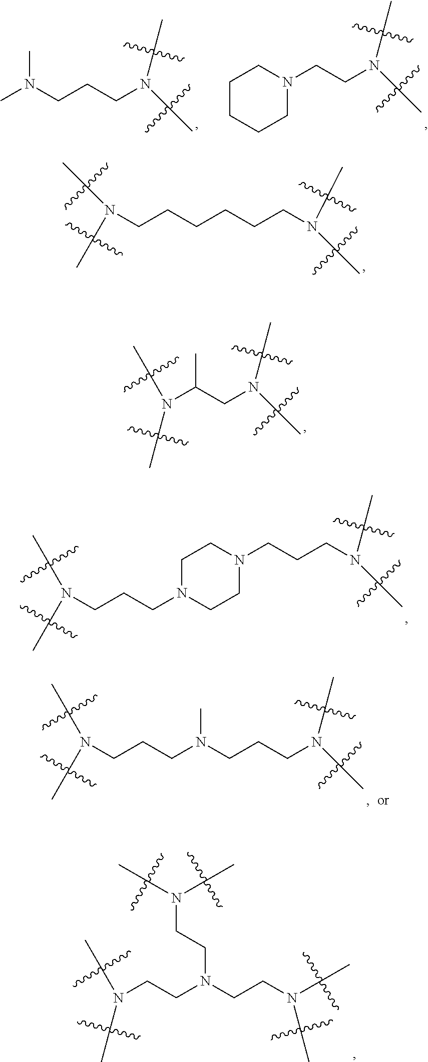

and

A″ is:

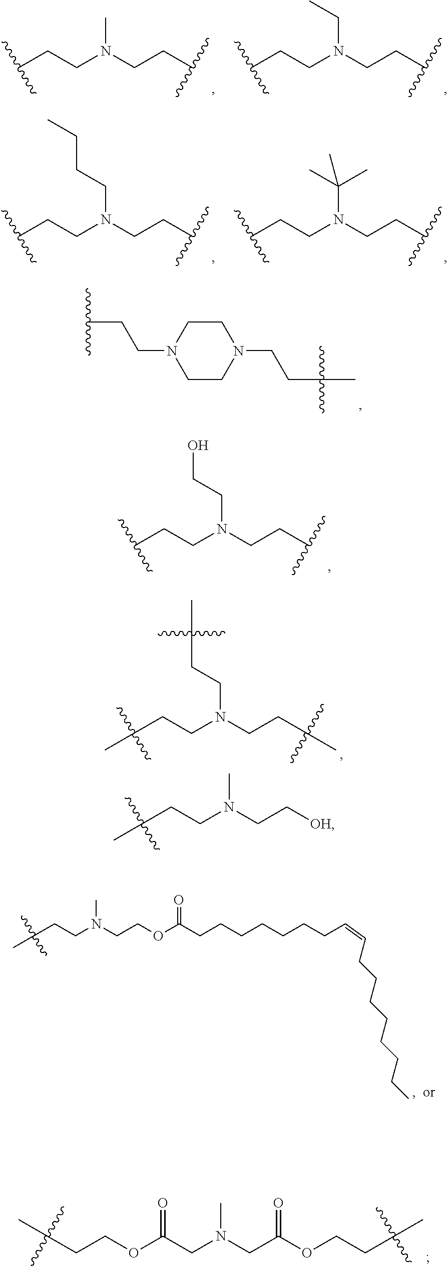

B is B′ or B″,

B′ is:

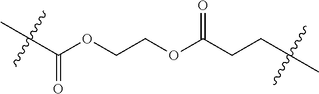 [SMILES: *CCC(=O)OCCOC(*)=O]

and

B″ is:

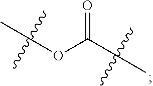 [SMILES: *OC(*)=O]

wherein when A is A′, then B is B′, and wherein when A is A″, then B is B″;

C is:

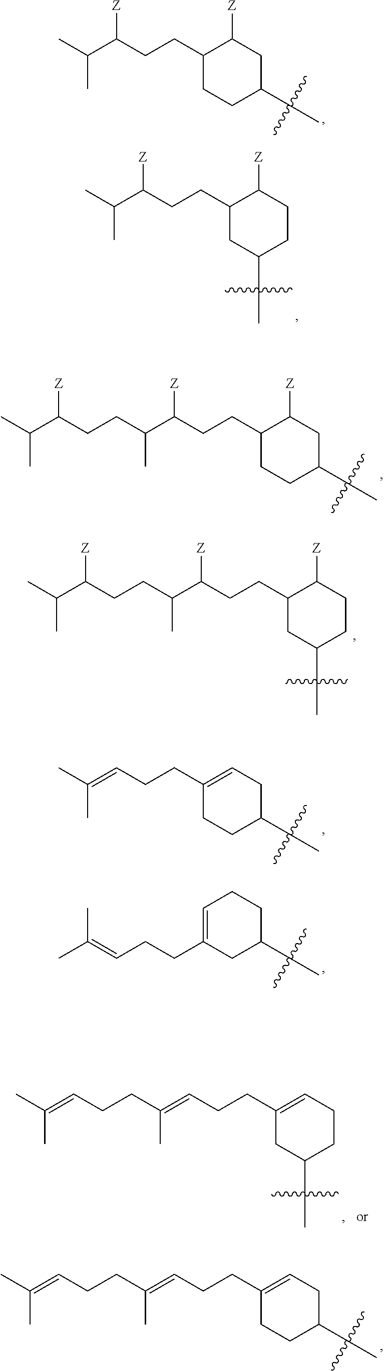

n is an integer between 1 to 6;

each Z is independently H or —S—R₁;

each R₁ is independently selected from: alkyl,

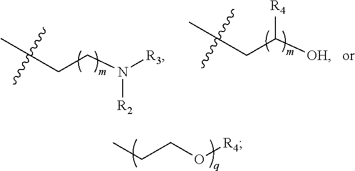

each R₂ or R₃ is independently selected from hydrogen, C₁-C₆ alkyl, C₁-C₆ alkenyl, aralkyl, or hydroxyalkyl;

each R₄ is hydrogen or C₁-C₃ alkyl;

each R₅ is

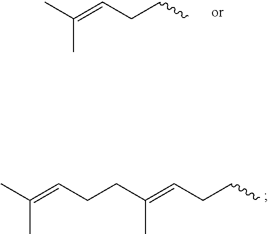

each R₆ is

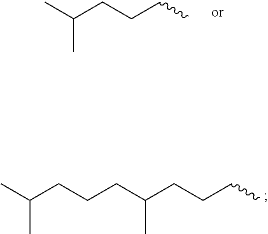

each m is an integer independently selected from 1-10; and

each q is an integer independently selected from 1-200.

In one aspect, the invention provides the following compounds:

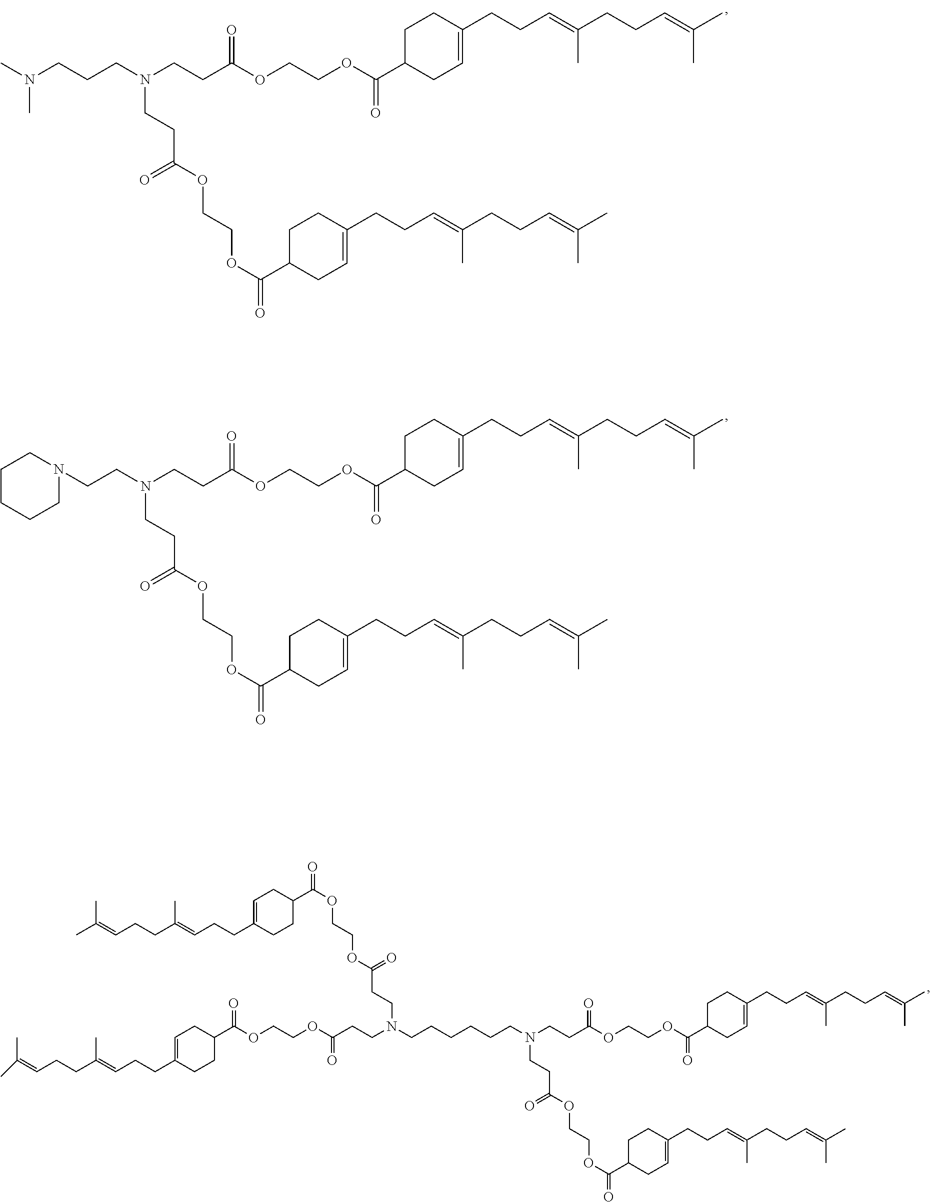

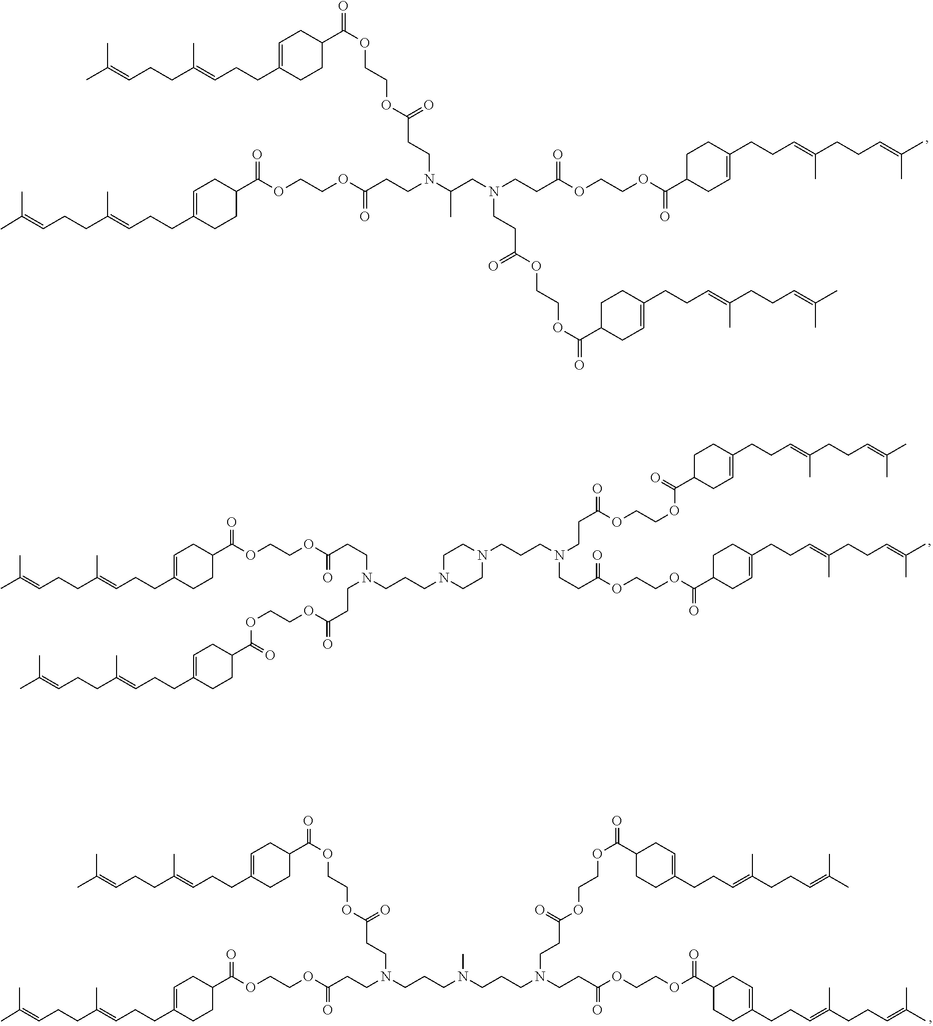

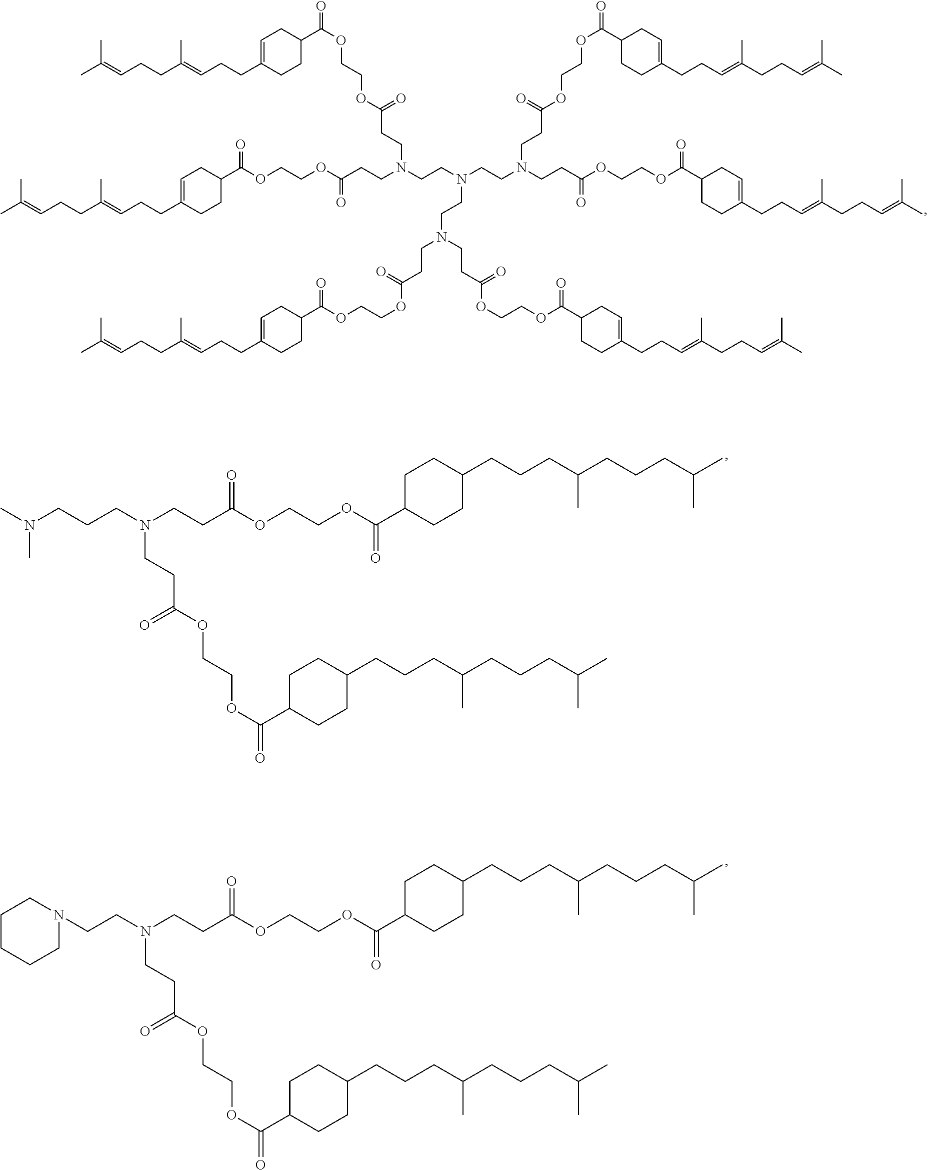

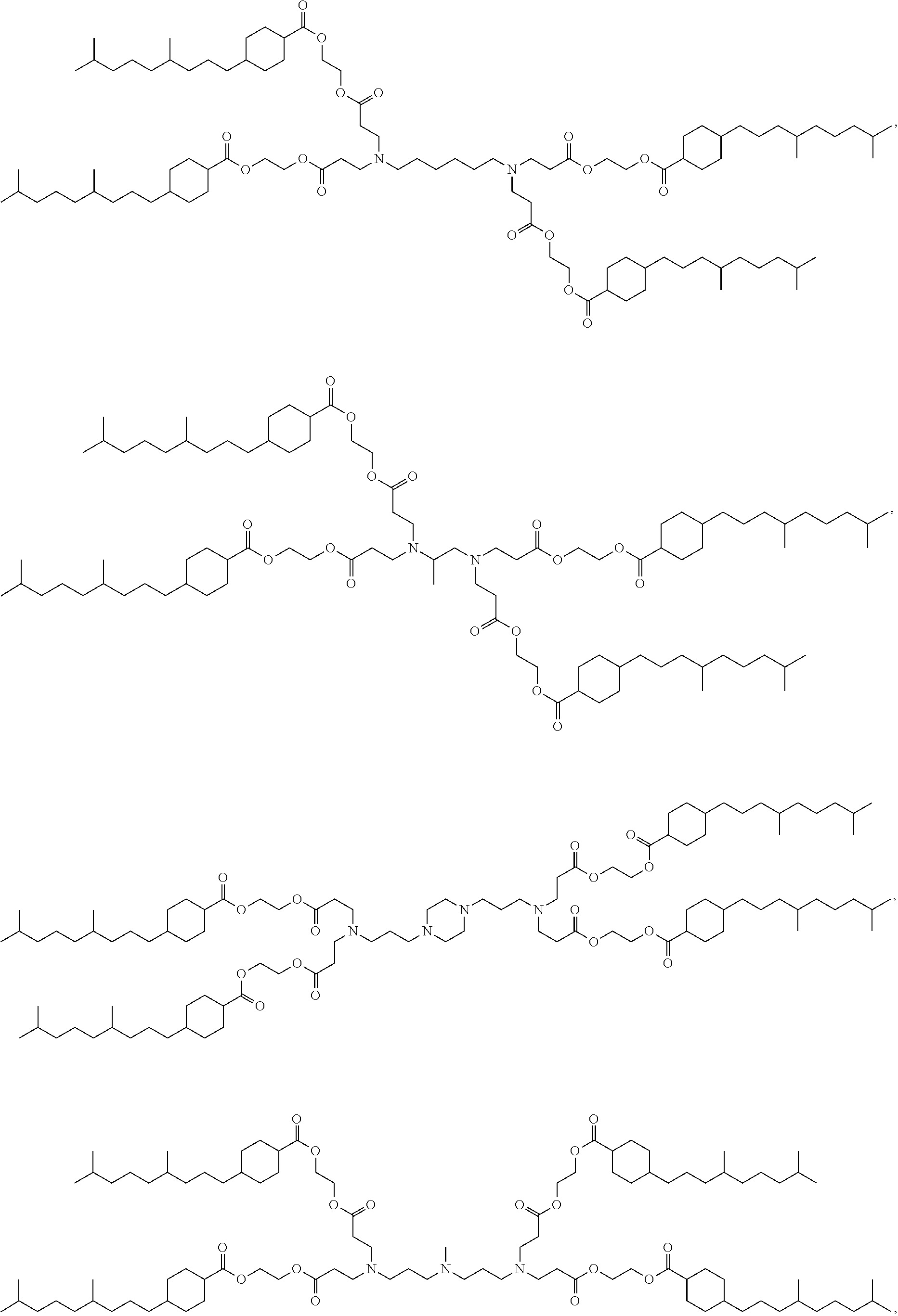

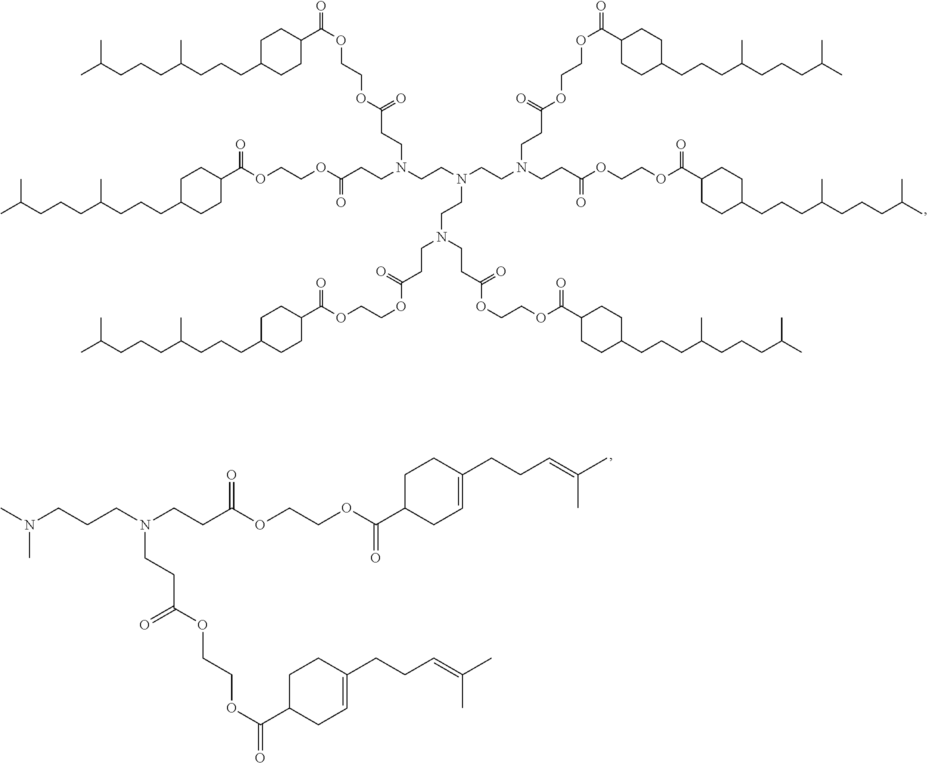

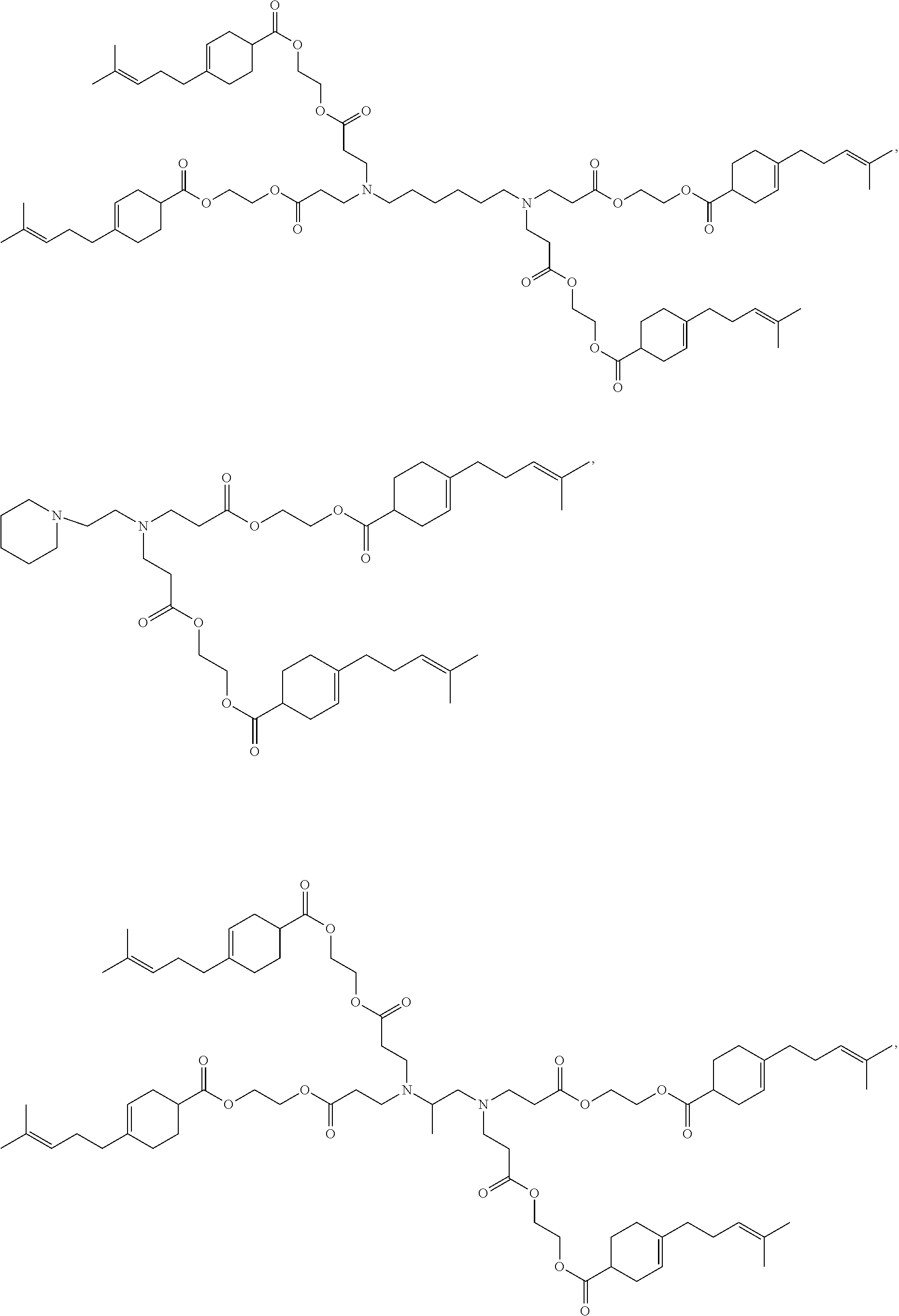

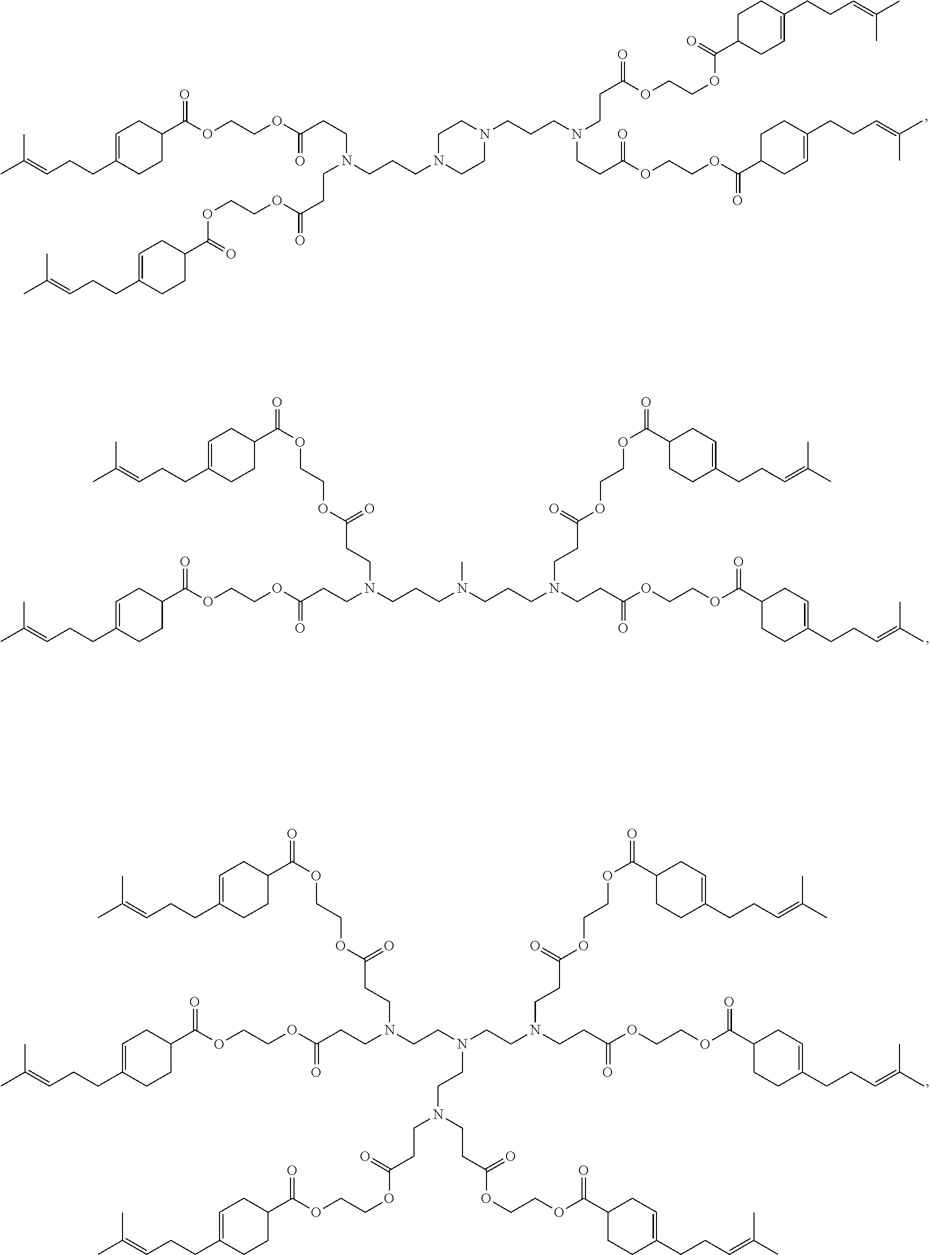

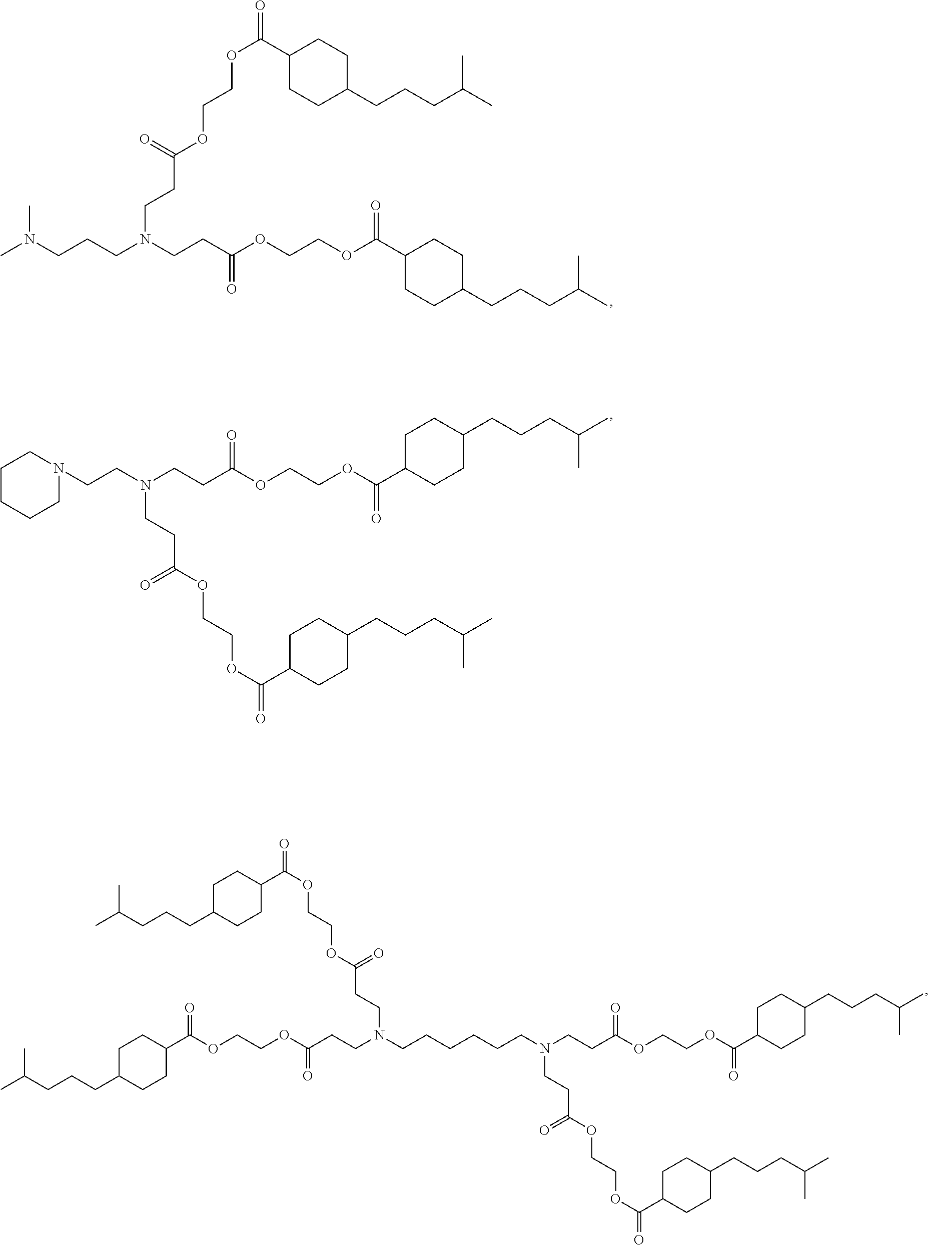

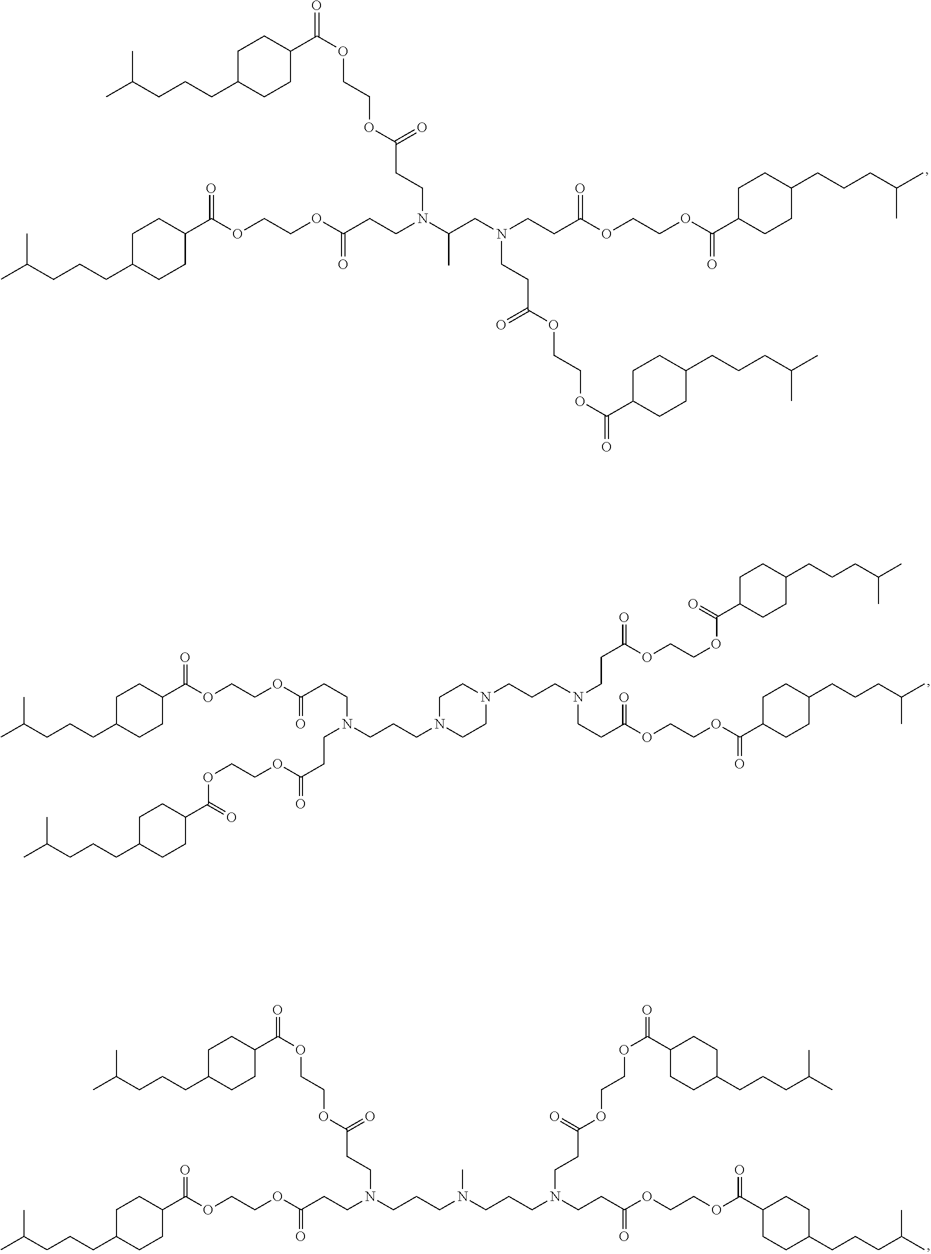

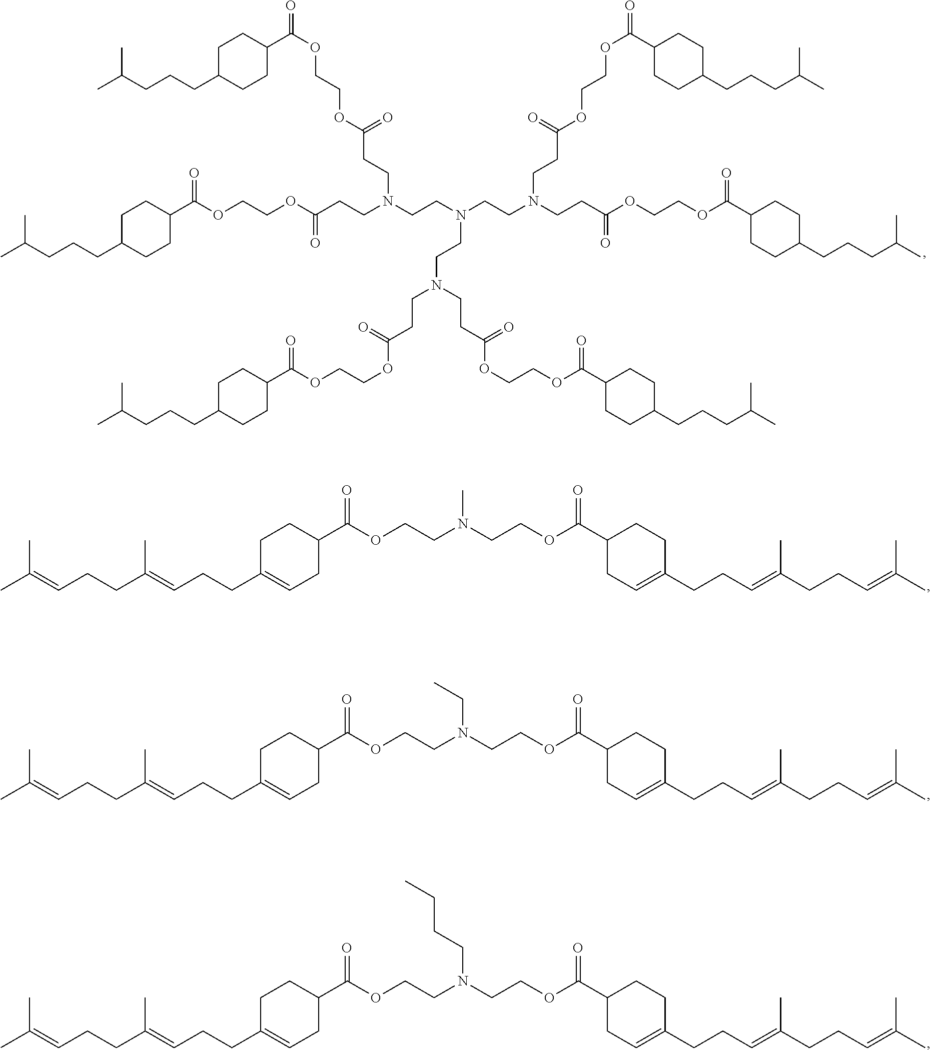

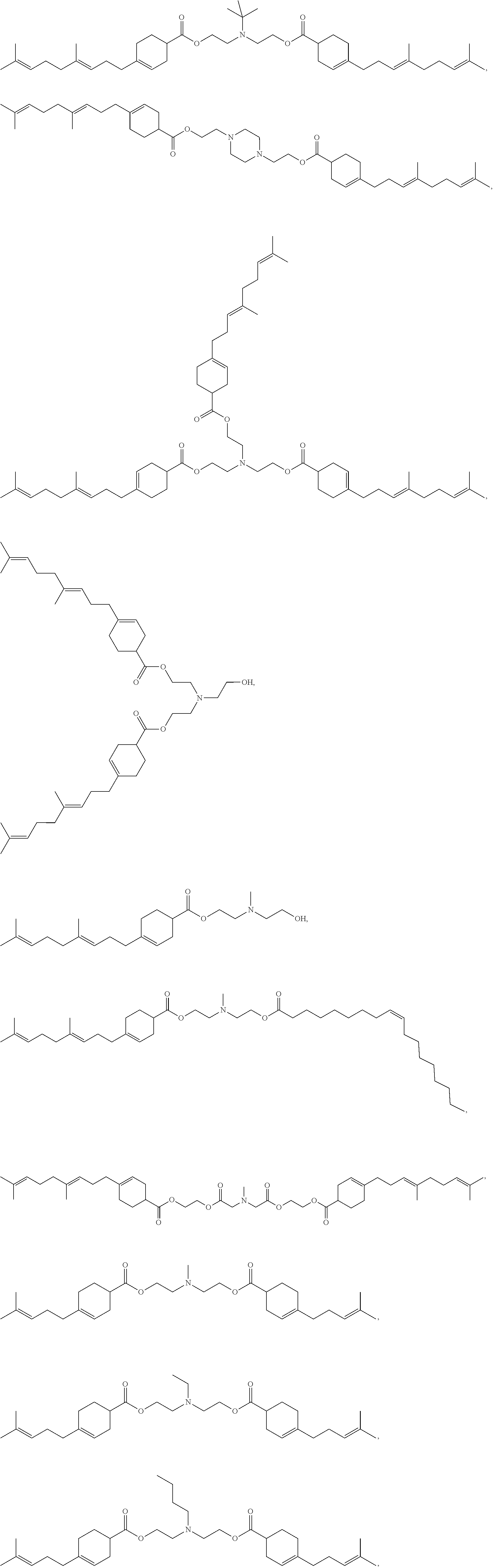

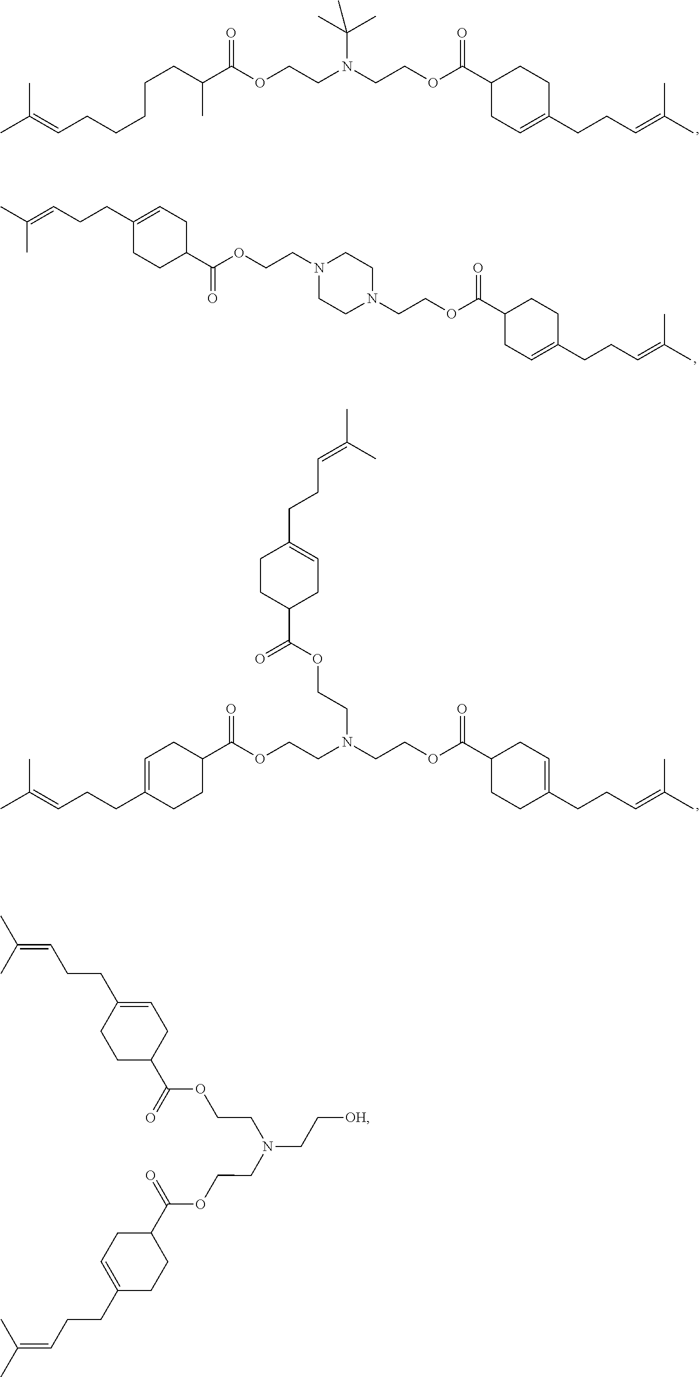

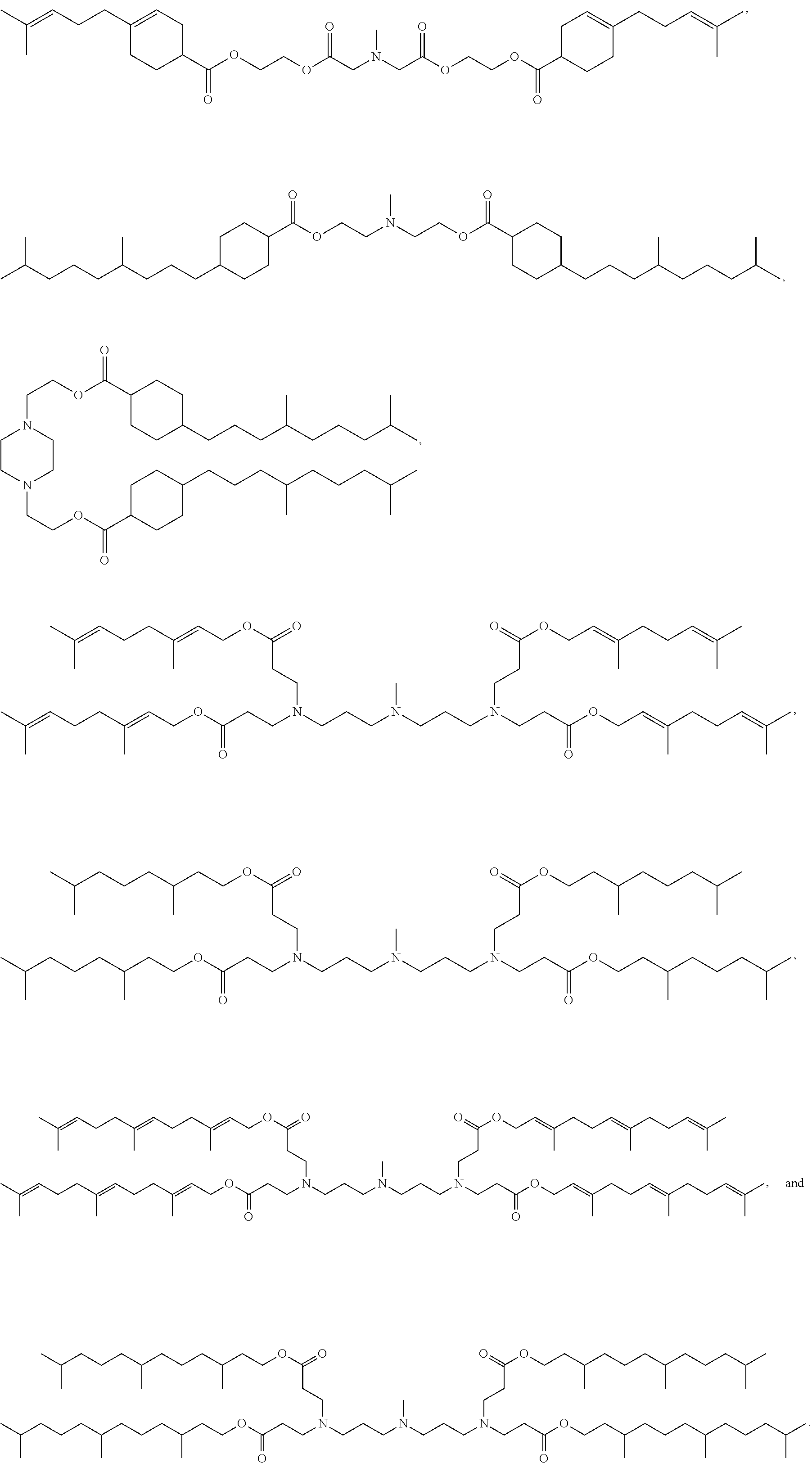

In addition to the structural formulas shown above, the exemplified compounds include compounds which are 1,3 isomers of the exemplified compounds (which are 1,4 isomers). In other words, all exemplified compounds include versions of the same compounds where the C unit is a 1,3 isomer. For example, when the invention describes compound:

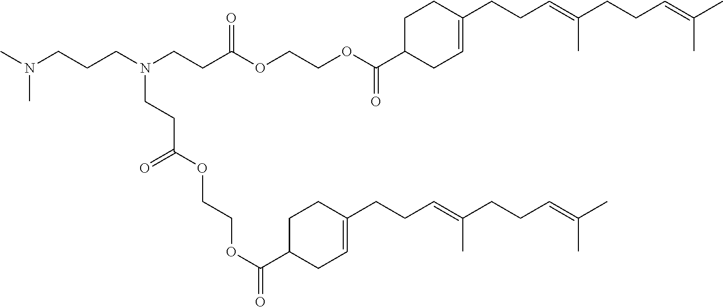 [SMILES: CC(C)=CCC/C(C)=C/CCC1=CCC(C(=O)OCCOC(=O)CCN(CCCN(C)C)CCC(=O)OCCOC(=O)C2CC=C(CC/C=C(\C)CCC=C(C)C)CC2)CC1]

it is understood that it also exemplifies compound with the following structure:

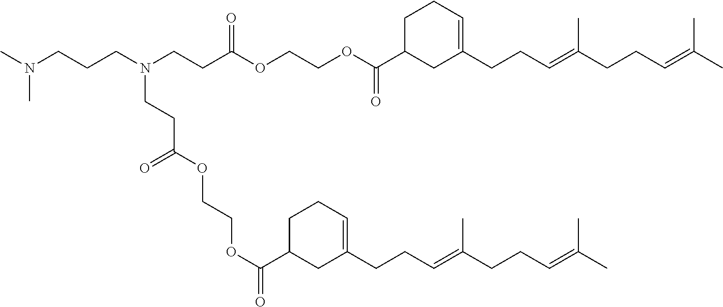 [SMILES: CC(C)=CCC/C(C)=C/CCC1=CCCC(C(=O)OCCOC(=O)CCN(CCCN(C)C)CCC(=O)OCCOC(=O)C2CCC=C(CC/C=C(\C)CCC=C(C)C)C2)C1]

In fact, usually the exemplified compounds exist as mixtures of 1,4 isomers and 1,3 isomers.

In one aspect, the compositions can be a lipoplex composition comprising a compound of Formula (I), Formula (II) or Formula (III) and at least one nucleic acid.

In some aspects, the compositions can be pharmaceutical compositions comprising a compound of Formula (I), Formula (II) or Formula (III), at least one nucleic acid and one or more pharmaceutically-acceptable excipient or diluent.

In one aspect, the present compositions can be used to deliver at least one nucleic acid to at least one cell by contacting the at least one cell with the present composition.

Unless otherwise defined, all technical and scientific terms used herein have the same meaning as commonly understood by one of ordinary skill in the art to which this disclosure belongs. In the specification, the singular forms also include the plural unless the context clearly dictates otherwise. The terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The term “about” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Ranges which are described as being “between” two values include the indicated values.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present specification, including definitions, will control. In addition, the materials, methods, and examples are illustrative only and are not intended to be limiting. Other features and advantages of the disclosure will be apparent from the following detailed description and claims.

### DETAILED DESCRIPTION OF THE INVENTION

### Definitions

In the chemical formulas shown herein, the marking

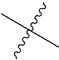

indicates the position where a functional group bonds to another portion of a molecule. As used herein, the term “alkyl” refers to straight and branched chain aliphatic groups having from 1 to 12 carbon atoms, As such, “alkyl” encompasses C₁, C₂, C₃, C₄, C₅, C₆, C₇, C₈, C₉, C₁₀, C₁₁ and C₁₂ groups. For instance, a C₁-C₆ alkyl group includes alkyl groups having 1 to 6 carbons. Examples of alkyl groups include, without limitation, methyl, ethyl, propyl, isopropyl, butyl, isobutyl, sec-butyl, tert-butyl, pentyl, and hexyl.

As used herein, the term “alkylene” is an alkyl, as defined hereinabove, that is positioned between and serves to connect two other chemical groups. Examples of alkylene groups include, without limitation, methylene, ethylene, propylene, butylene, pentylene and hexylene.

As used herein, the term “alkenyl” refers to an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

As used herein, the term “aralkyl” group comprises an aryl group covalently linked to an alkyl group, either of which can independently be optionally substituted or unsubstituted. An example of an aralkyl group is —(C₁-C₆)alkyl(C₆-C₁₀)aryl, including, without limitation, benzyl, phenethyl, and naphthylmethyl.

As used herein, the term “aryl” group is a C₆-C₁₄ aromatic moiety comprising one to three aromatic rings, which is optionally substituted. As such, “aryl” includes C₆, C₇, C₈, C₉, C₁₀, C₁₁, C₁₂ C₁₃, and C₁₄ cyclic hydrocarbon groups. An exemplary aryl group is a C.sub.6-C.sub.10 aryl group. Particular aryl groups include, without limitation, phenyl, naphthyl, anthracenyl, and fluorenyl.

As used herein, the term “C2-C100 hydrocarbon chain” refers to straight or branched chain, saturated or unsaturated comprising 2 to 100 carbon atoms. Examples of C2-C100 hydrocarbon chains groups include ethyl, propyl, isopropyl, butyl, isobutyl, sec-butyl, tert-butyl, pentyl, hexyl, heptanyl, octanyl, nonanyl, decanyl, undecanyl, dodecanyl, tridecanyl, icosanyl, triacontanyl, and tetracontanyl, and unsaturated counterparts thereof, e.g., propenyl and propynyl counterparts of propyl.

As used herein, the term “cycloalkyl” as employed herein includes saturated and partially unsaturated cyclic hydrocarbon groups having 3 to 12 carbons. As such, “cycloalkyl” includes C₃, C₄, C₅, C₆, C₇, C₈, C₉, C₁₀, C₁₁ and C₁₂ cyclic hydrocarbon groups. Representative cycloalkyl groups include, without limitation, cyclopropyl, cyclobutyl, cyclopentyl, cyclopentenyl, cyclohexyl, cyclohexenyl, cycloheptyl, and cyclooctyl.

As used herein, the terms “halo” and “halogen” refer to fluoro, chloro, bromo and iodo.

As used herein, the term “hydroxyalkyl” refers to -alkyl-OH or an alkyl chain substituted with at least one —OH.

### Compositions of the Present Disclosure—Terpene-Derived Cationic Lipidoids

### Compounds

In one aspect, the present disclosure provides novel terpene-derived cationic lipidoid compounds of Formula (I).

In one aspect, the terpene-derived lipidoid compounds are represented by Formula (I):

A-(B—C)ₙ Formula (I)

wherein:

A is A′ or A″,

A′ is:

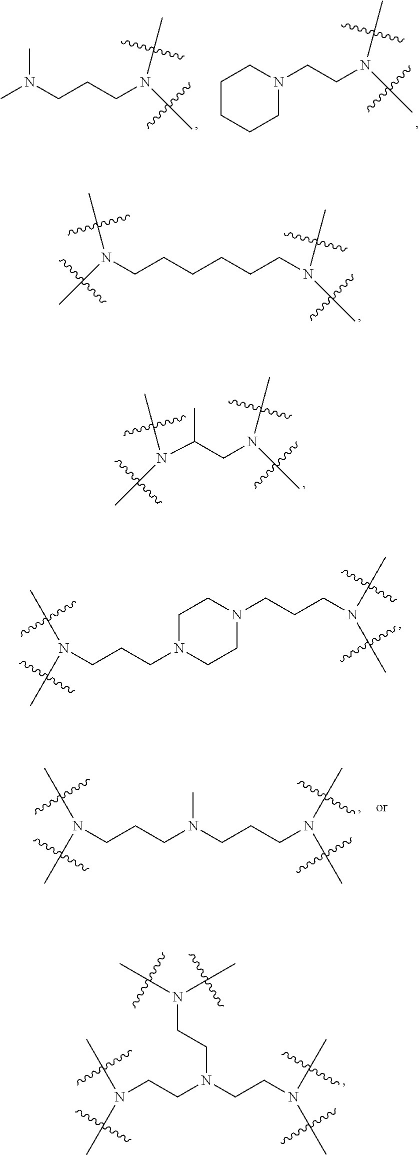

and

A″ is:

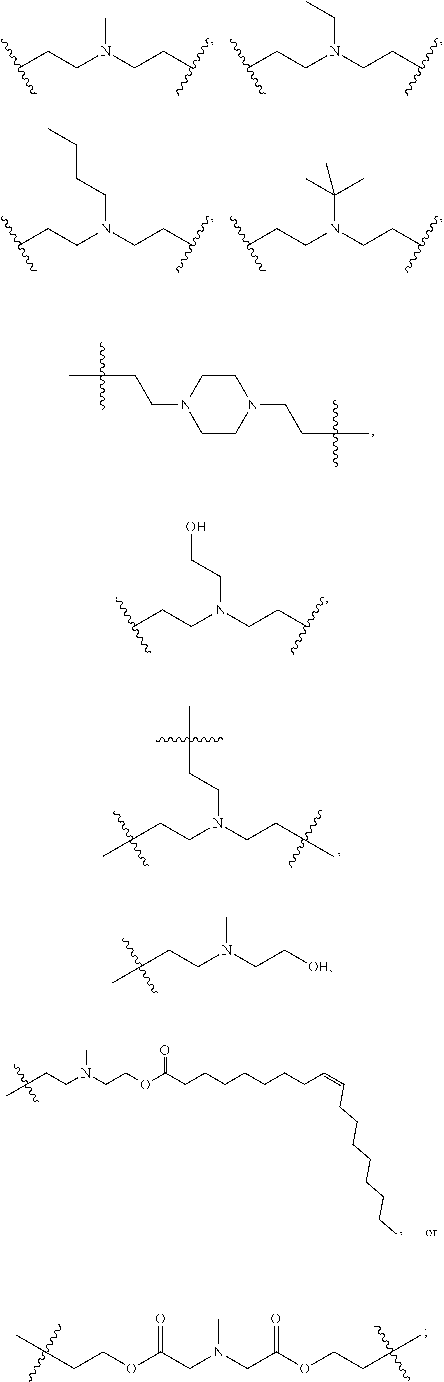

B is B′ or B″,

B′ is:

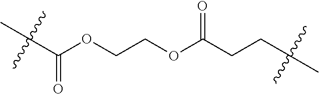 [SMILES: *CCC(=O)OCCOC(*)=O]

and

B′ is:

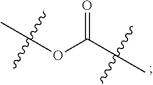 [SMILES: *OC(*)=O]

wherein when A is A′, then B is B′, and wherein when A is A″, then B is B″;

C is:

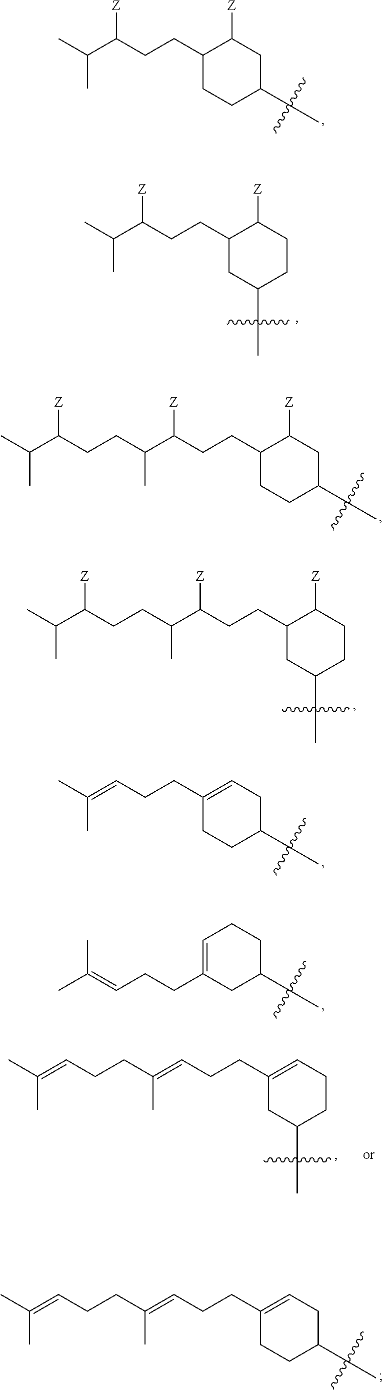

n is an integer between 1 to 6;

each Z is independently H or —S—R₁;

each R₁ is independently selected from: alkyl,

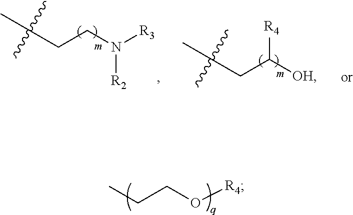

each R₂ or R₃ is independently selected from hydrogen, C₁-C₆ alkyl, C₁-C₆ alkenyl, aralkyl, or hydroxyalkyl;

each R₄ is hydrogen or C₁-C₃ alkyl;

each m is an integer independently selected from 1-10; and

each q is an integer independently selected from 1-200.

In some aspects, n is 1.

In some aspects, n is 2.

In some aspects, A is A′.

In some aspects, B is B′.

In some aspects, A′ is

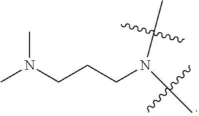 [SMILES: *N(*)CCCN(C)C]

In some aspects, A′ is

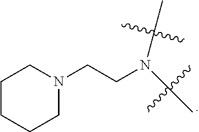 [SMILES: *N(*)CCN1CCCCC1]

In some aspects, A′ is

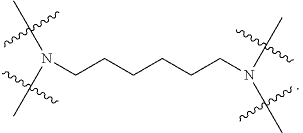 [SMILES: *N(*)CCCCCCN(*)*]

In some aspects, A′ is

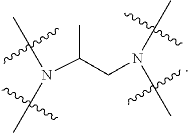 [SMILES: *N(*)CC(C)N(*)*]

In some aspects, A′ is

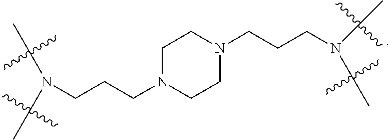 [SMILES: *N(*)CCCN1CCN(CCCN(*)*)CC1]

In some aspects, A′ is

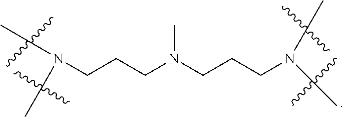 [SMILES: *N(*)CCCN(C)CCCN(*)*]

In some aspects, A′ is

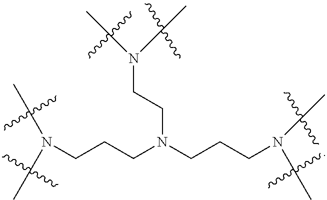 [SMILES: *N(*)CCCN(CCCN(*)*)CCN(*)*]

In some aspects, A″ is

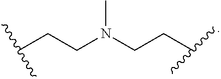 [SMILES: *CCN(C)CC*]

In some aspects, A″ is

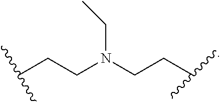 [SMILES: *CCN(CC)CC*]

In some aspects, A″ is

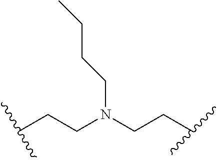 [SMILES: *CCN(CC*)CCCC]

In some aspects, A″ is

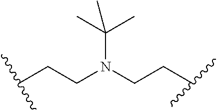 [SMILES: *CCN(CC*)C(C)(C)C]

In some aspects, A″ is

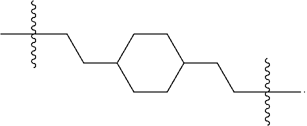 [SMILES: *CCC1CCC(CC*)CC1]

In some aspects, A″ is

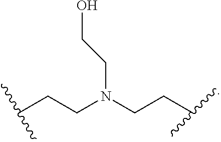 [SMILES: *CCN(CC*)CCO]

In some aspects, A″ is

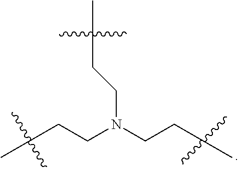 [SMILES: *CCN(CC*)CC*]

In some aspects, A″ is

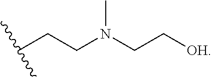 [SMILES: *CCN(C)CCO]

In some aspects, A″ is

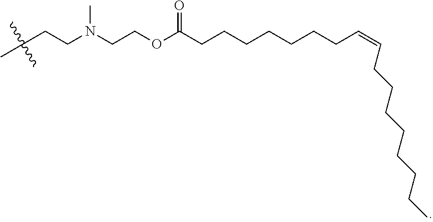 [SMILES: *CCN(C)CCOC(=O)CCCCCCC/C=C\CCCCCCCC]

In some aspects, A″ is

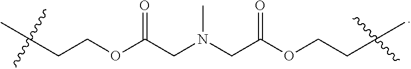 [SMILES: *CCOC(=O)CN(C)CC(=O)OCC*]

In some aspects, B″ is B′.

In some aspects, B is B″.

In some aspects, B′ is

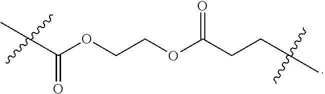 [SMILES: *CCC(=O)OCCOC(*)=O]

In some aspects, B″ is

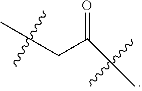 [SMILES: *CC(*)=O]

In some aspects, the carbonyl group of B′ or B″ is attached to “C” part of the molecule and the other end of B′ or B″ is attached to “A” part of the molecule.

In some aspects, each C segment is:

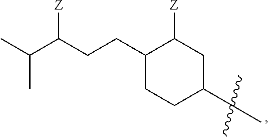

In some aspects, each C segment is:

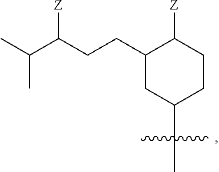

In some aspects, each C segment is:

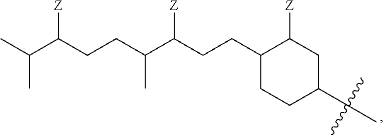

In some aspects, each C segment is:

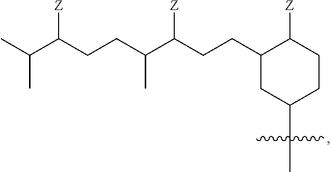

In some aspects, each C segment is:

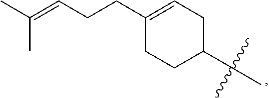 [SMILES: *C1CC=C(CCC=C(C)C)CC1]

In some aspects, each C segment is:

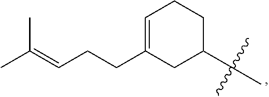 [SMILES: *C1CCC=C(CCC=C(C)C)C1]

In some aspects, each C segment is:

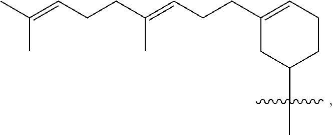 [SMILES: *C1CCC=C(CC/C=C(\C)CCC=C(C)C)C1]

In some aspects, each C segment is:

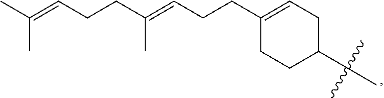 [SMILES: *C1CC=C(CC/C=C(\C)CCC=C(C)C)CC1]

In some aspects, at least one, at least two, or at least three, or at least four or more Z groups of the C segment is —S—R₁, wherein R₁ is alkyl.

In one such embodiment, m is one and R₂, and R₃ are each independently C₁-C₆ alkyl. In one embodiment, each of the C₁-C₆ alkyl groups is methyl. In this embodiment, 2-(dimethylamino)ethanethiol or 2-(dimethylamino)ethanethiol hydrochloride can be used as the derivatizing agent.

In some aspects, at least one, at least two, or at least three, or at least four or more Z groups of the C segment is —S—R₁, wherein R₁ is

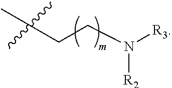

In one such embodiment, m is one and R₂, and R₃ are each independently C₁-C₆ alkyl. In one embodiment, each of the C₁-C₆ alkyl groups is methyl or ethyl. In this embodiment, 2-(dimethylamino)ethanethiol or 2-(dimethylamino)ethanethiol hydrochloride can be used as the derivatizing agent. In the case where each of the C1-C6 alkyl groups are ethyl, 2-diethylaminoethane thiol or 2-diethylaminoethane thiol hydrochloride can be used as the derivatizing agent.

In some aspects, each Z is —S—R₁, wherein at least one, at least two, or at least three, or at least four or more R₁ is:

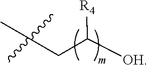

In one embodiment, m is one and R₄ is hydrogen. In this embodiment, ethane thiol can be used as the derivatizing agent.

In some aspects, each Z is —S—R₁, wherein at least one, at least two, or at least three, or at least four or more R₁ is:

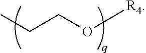

In one embodiment, q is one and R₄ is methyl.

In some aspects, the terpene-derived cationic lipidoid compound of Formula (I) is a compound selected from the following:

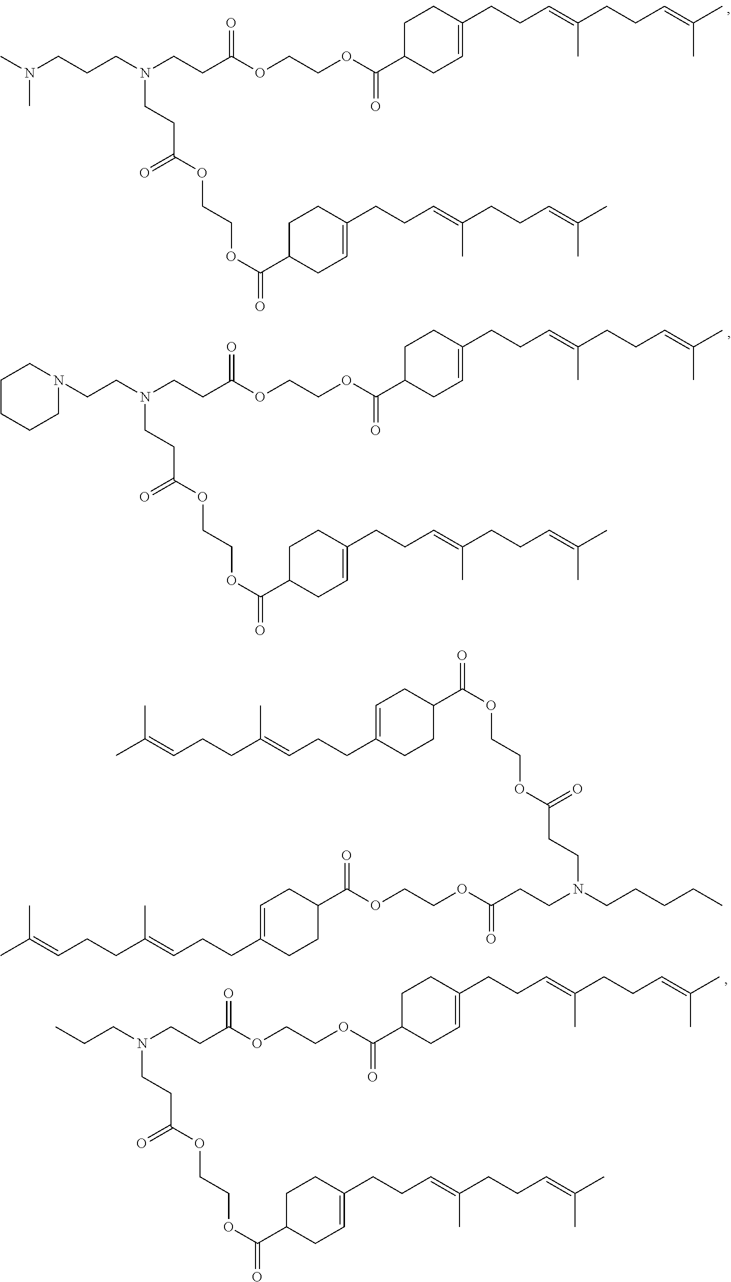

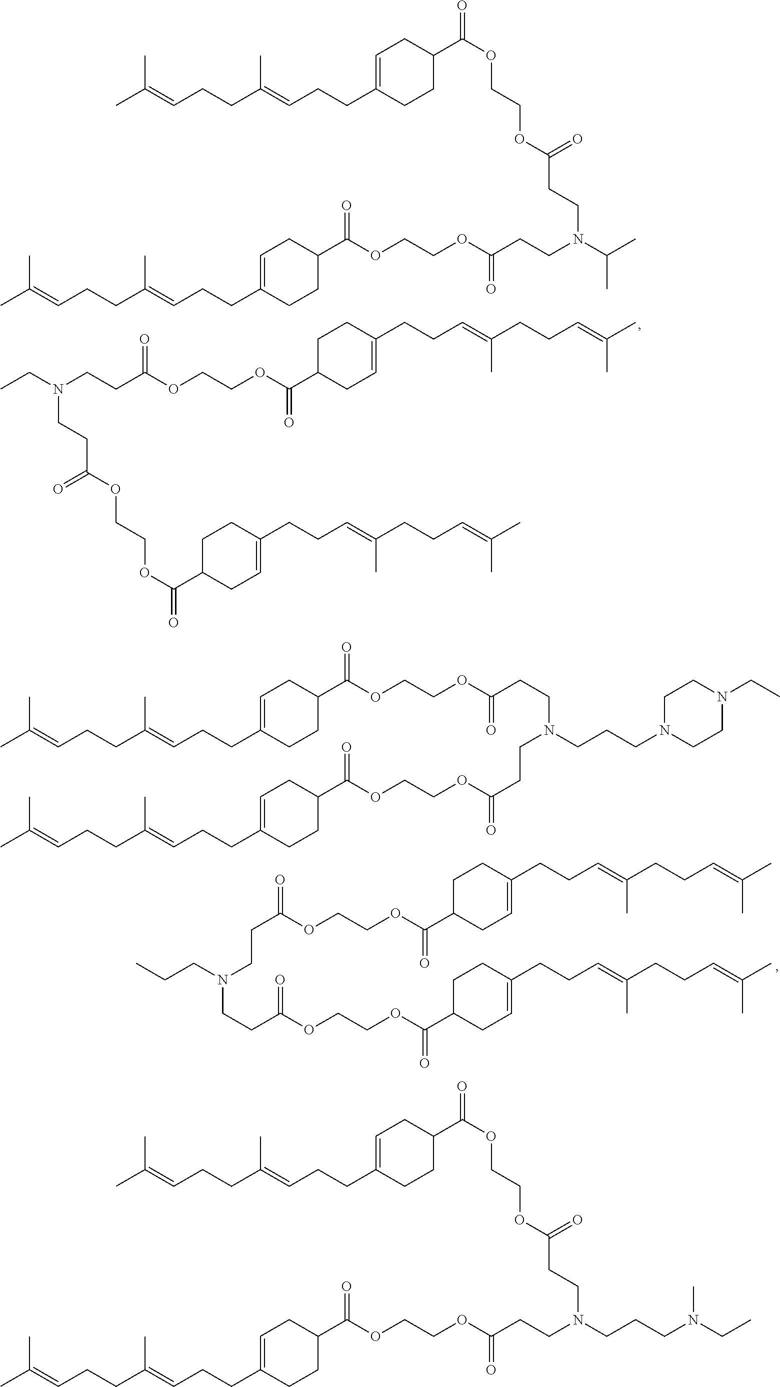

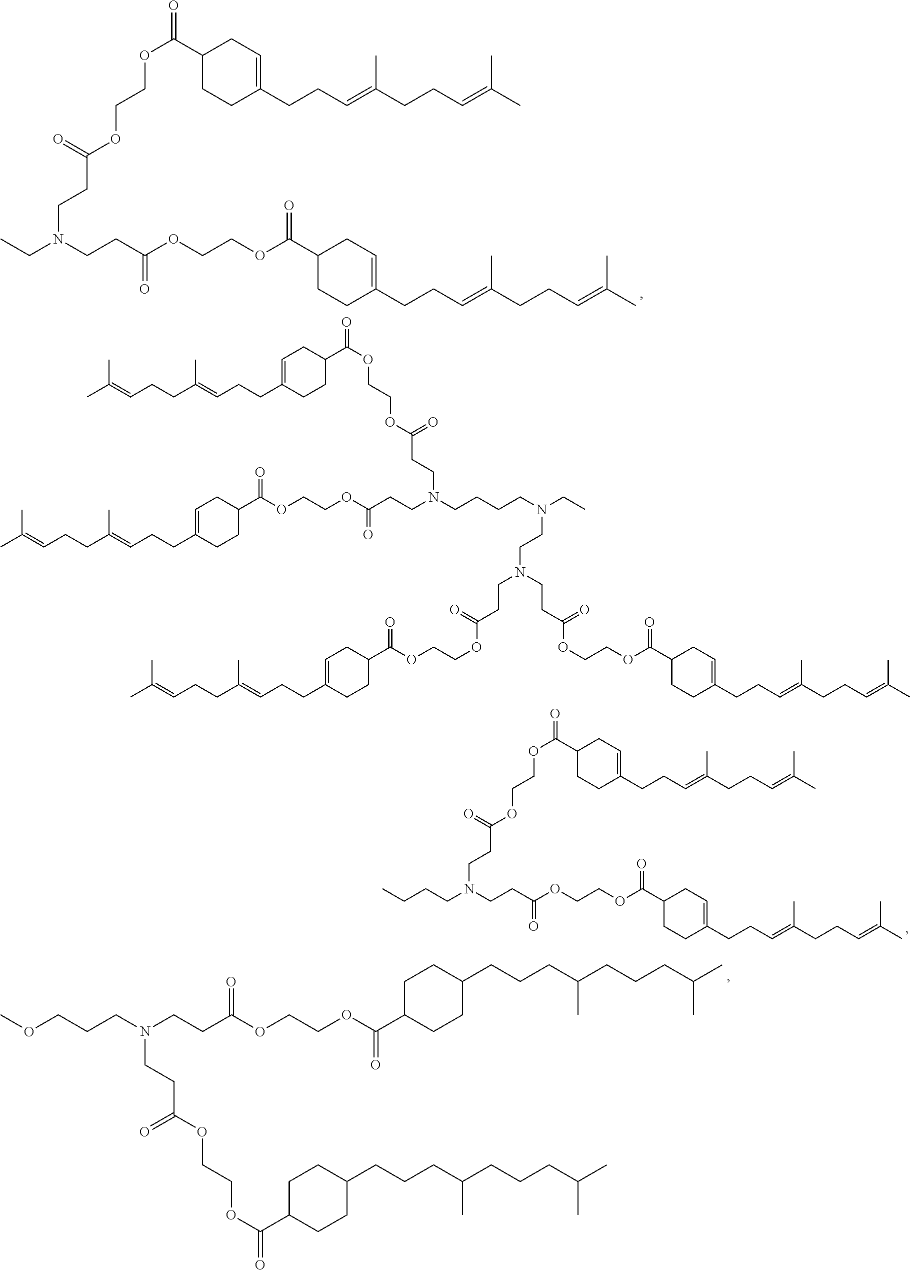

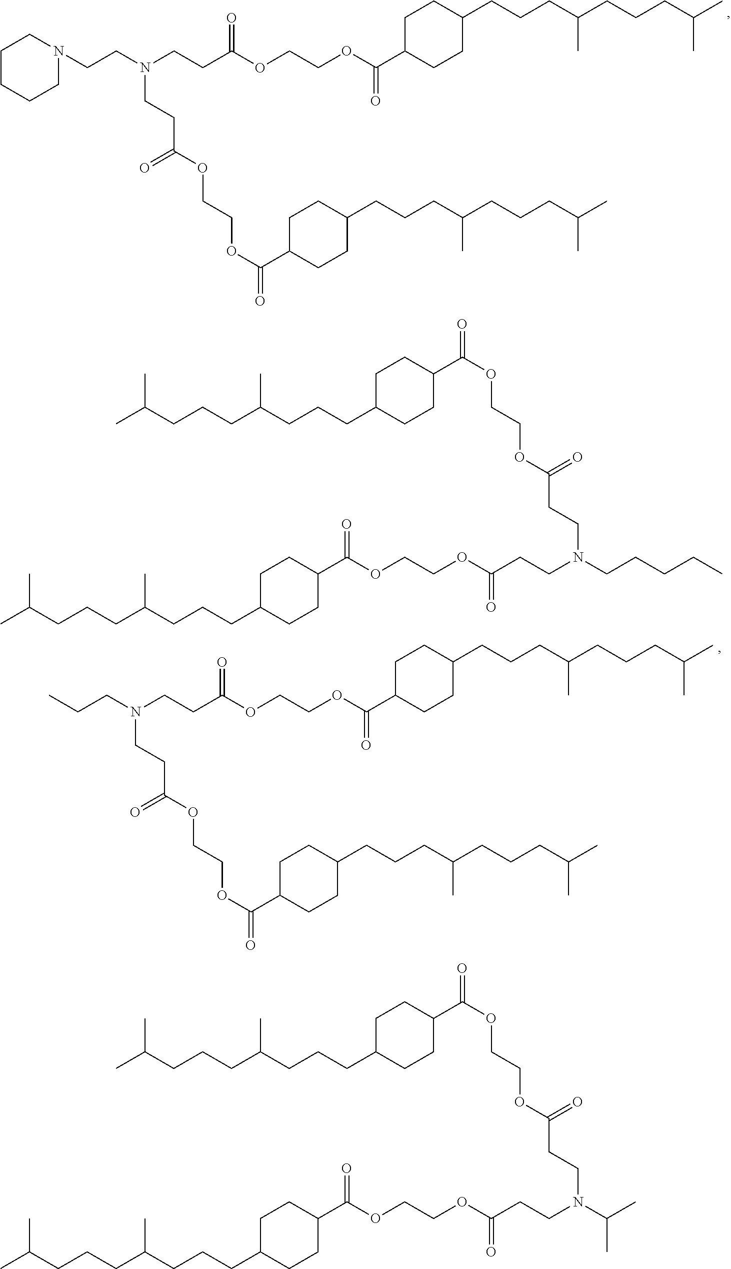

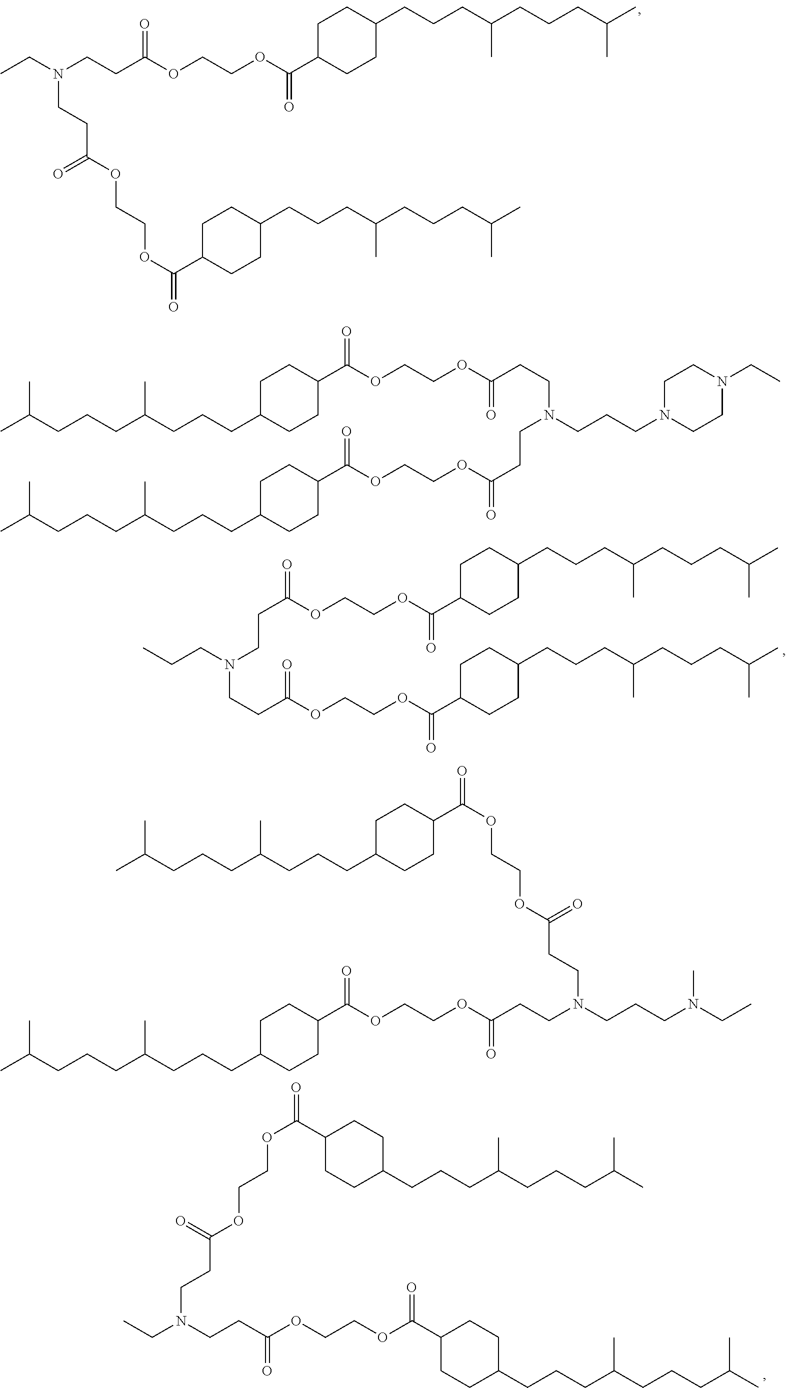

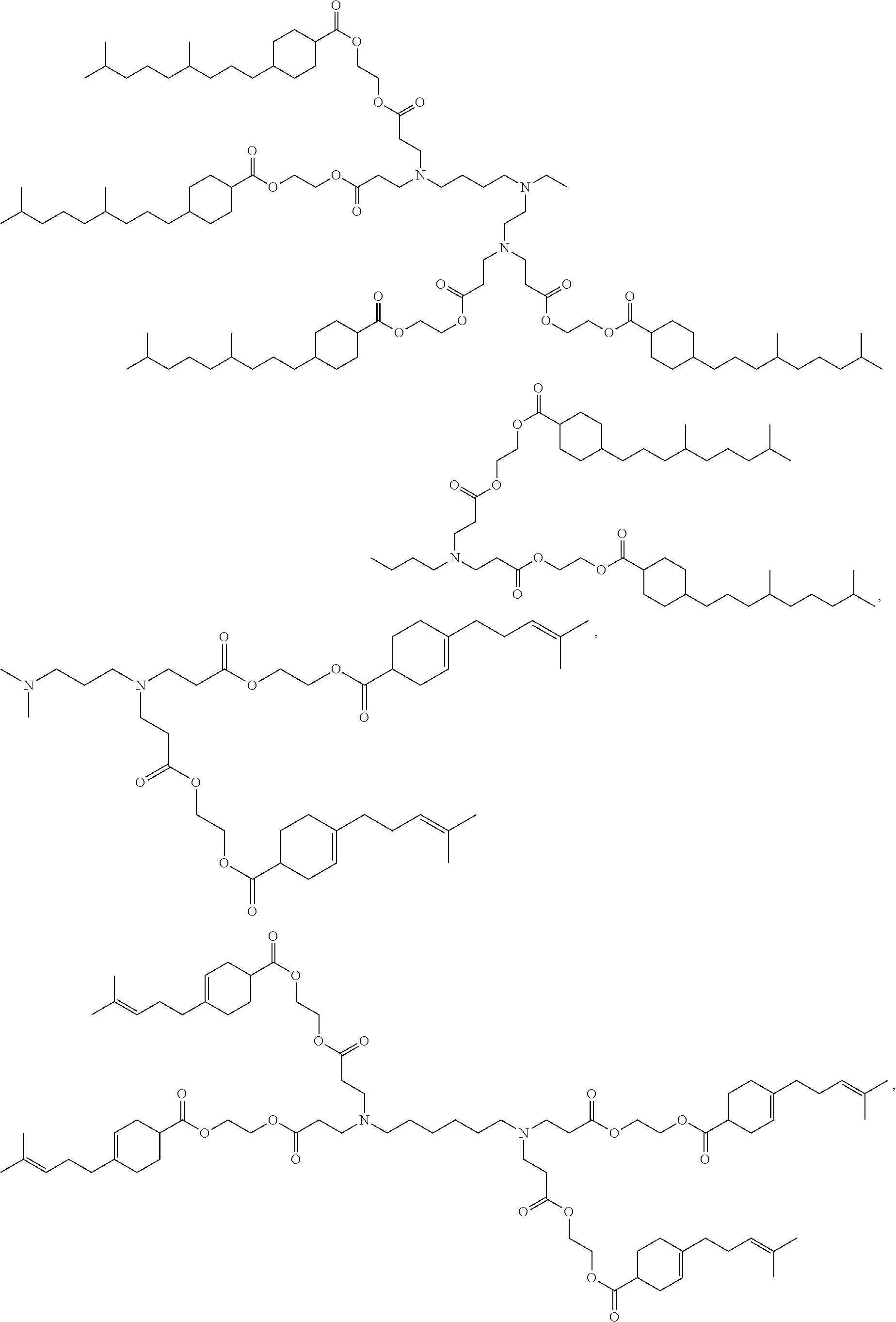

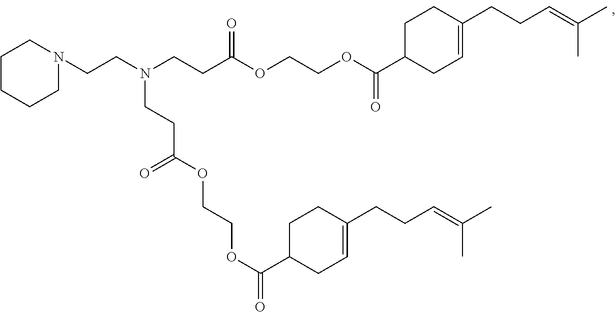 [SMILES: CC(C)=CCCC1=CCC(C(=O)OCCOC(=O)CCN(CCC(=O)OCCOC(=O)C2CC=C(CCC=C(C)C)CC2)CCN2CCCCC2)CC1]

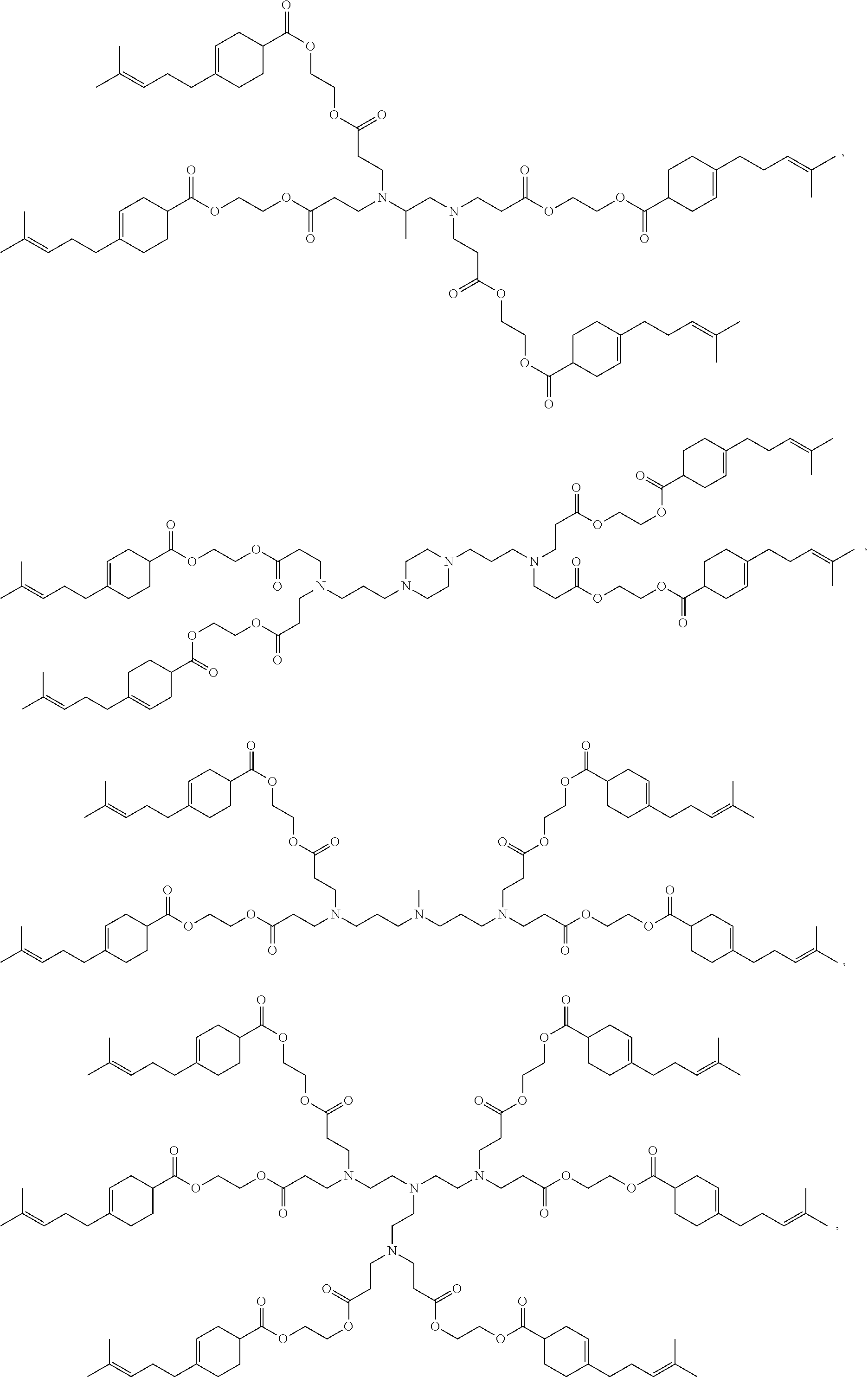

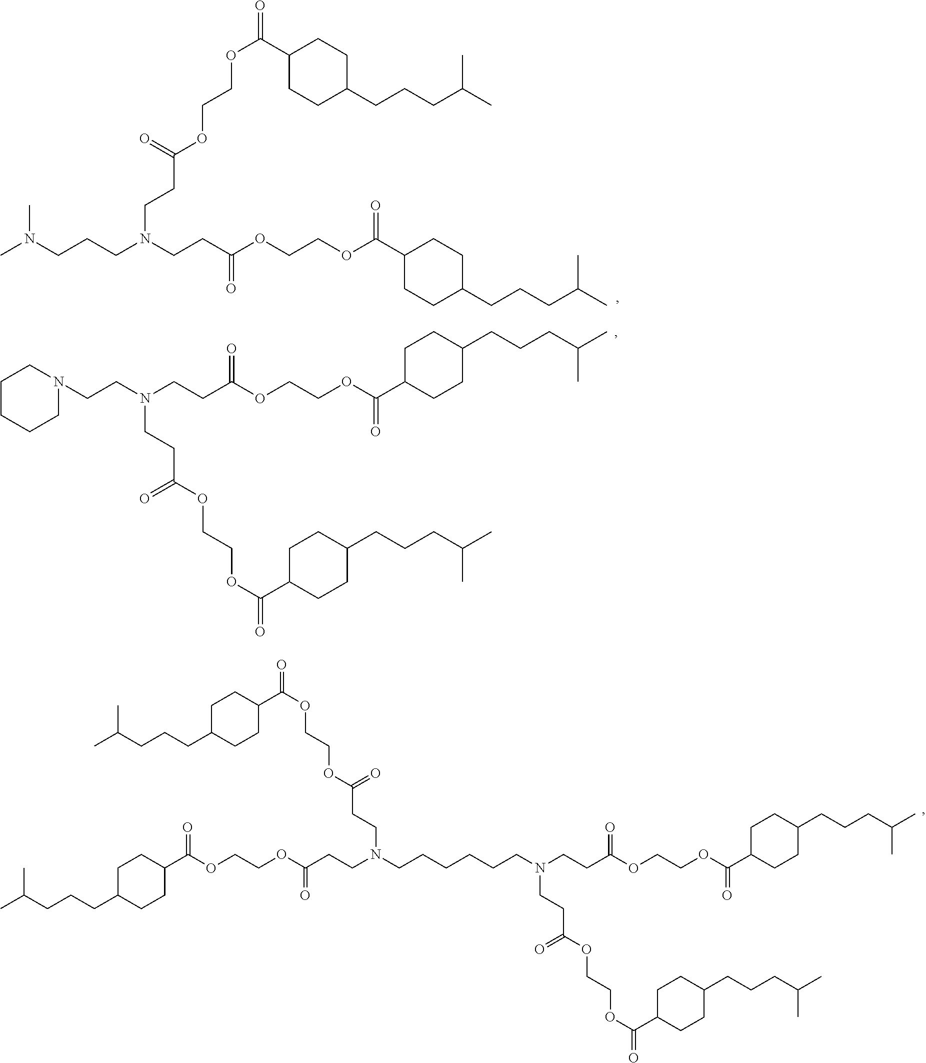

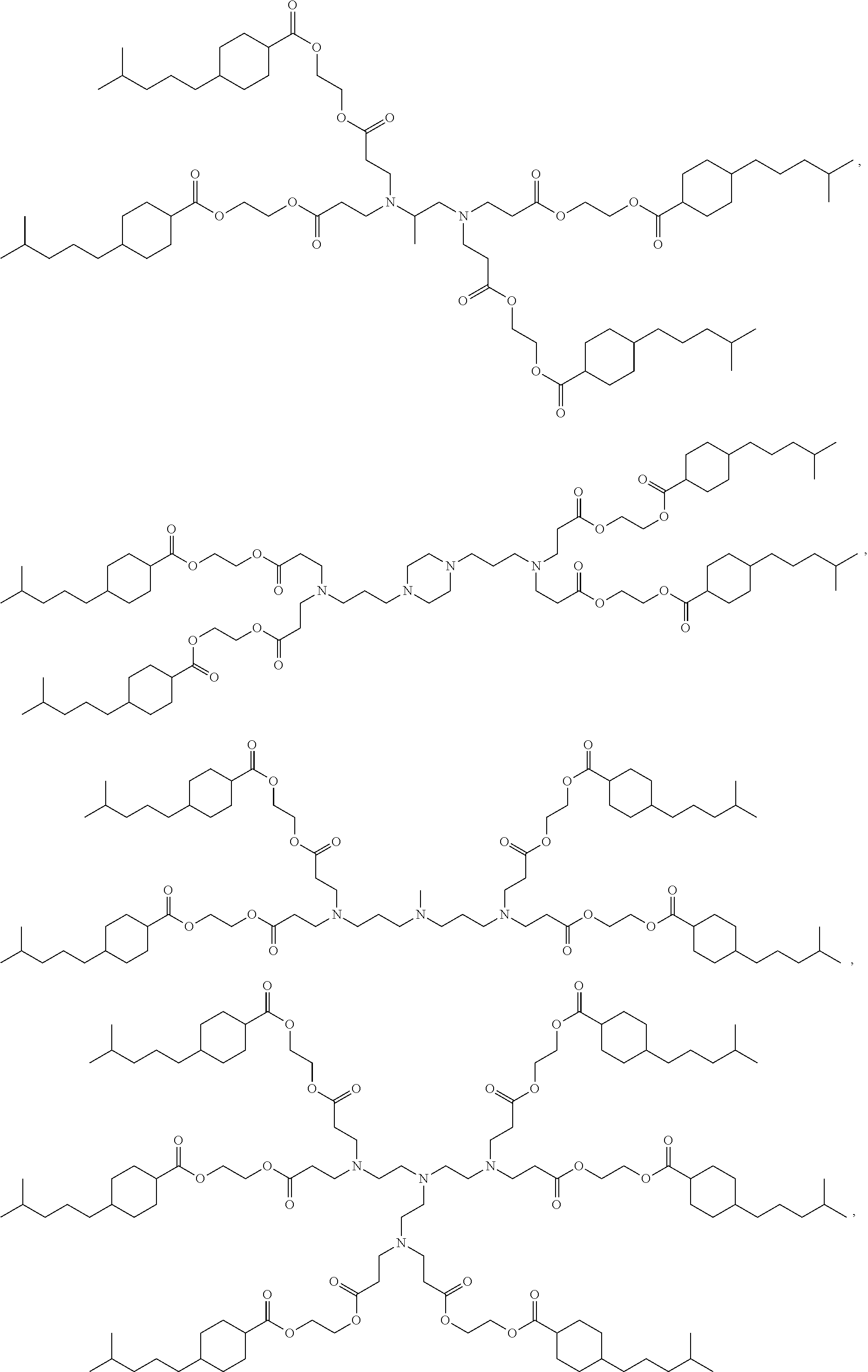

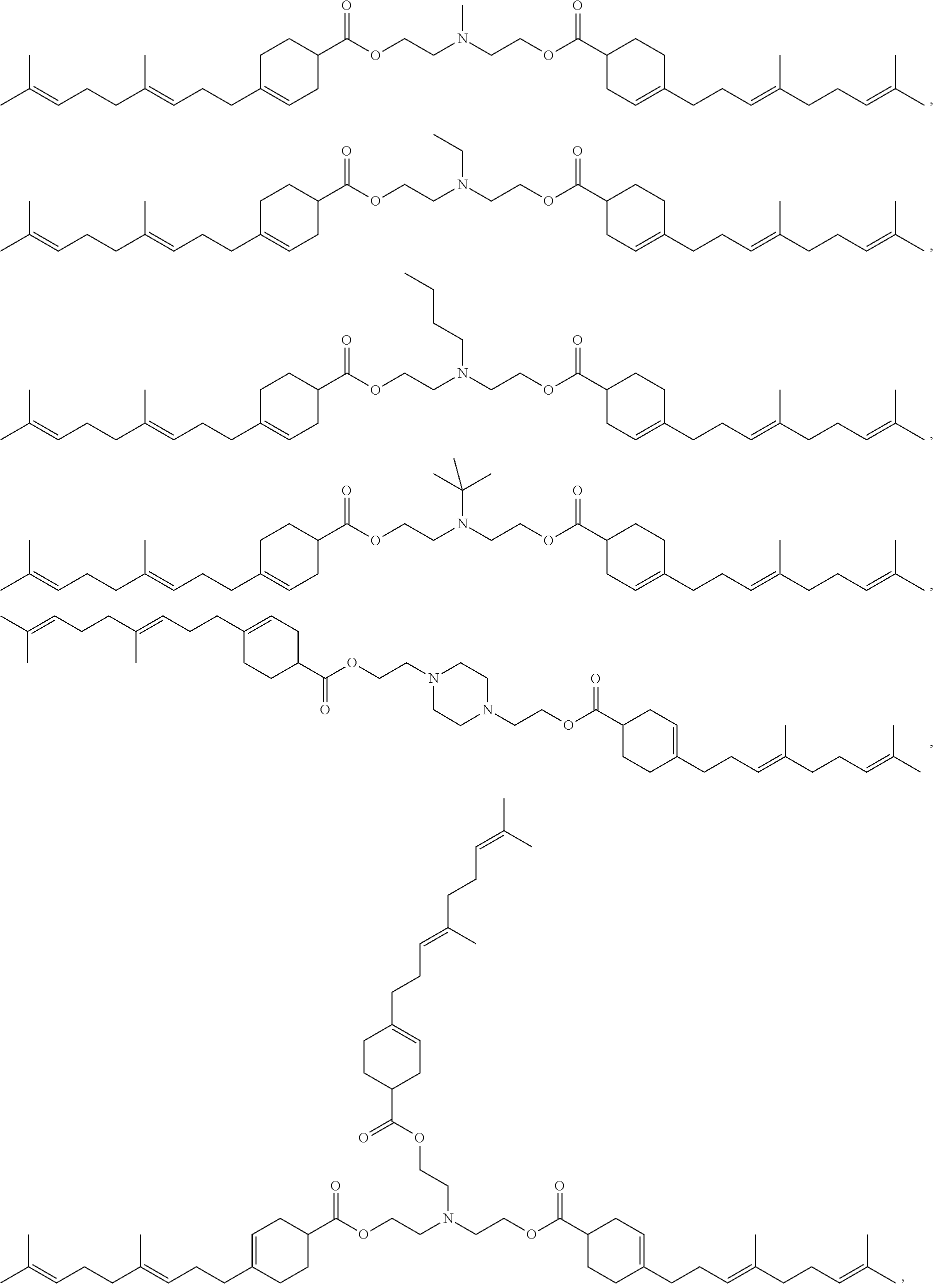

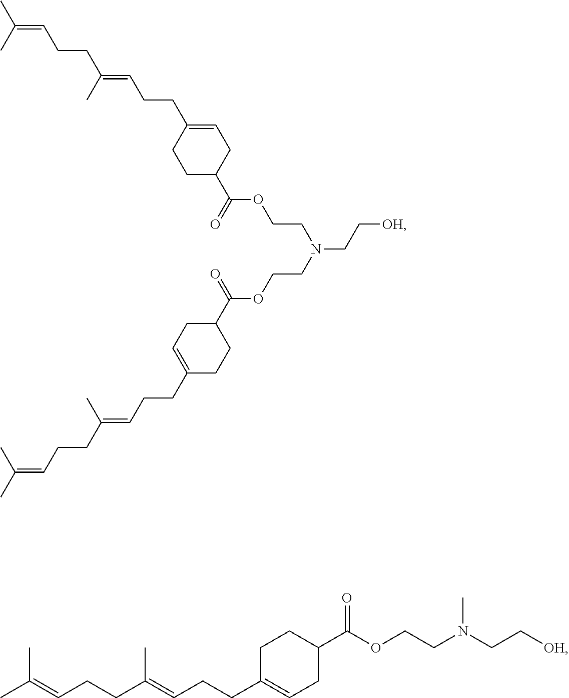 [SMILES: CC(C)=CCC/C(C)=C/CCC1=CCC(C(=O)OCCN(CCO)CCOC(=O)C2CC=C(CC/C=C(\C)CCC=C(C)C)CC2)CC1 ; CC(C)=CCC/C(C)=C/CCC1=CCC(C(=O)OCCN(C)CCO)CC1]

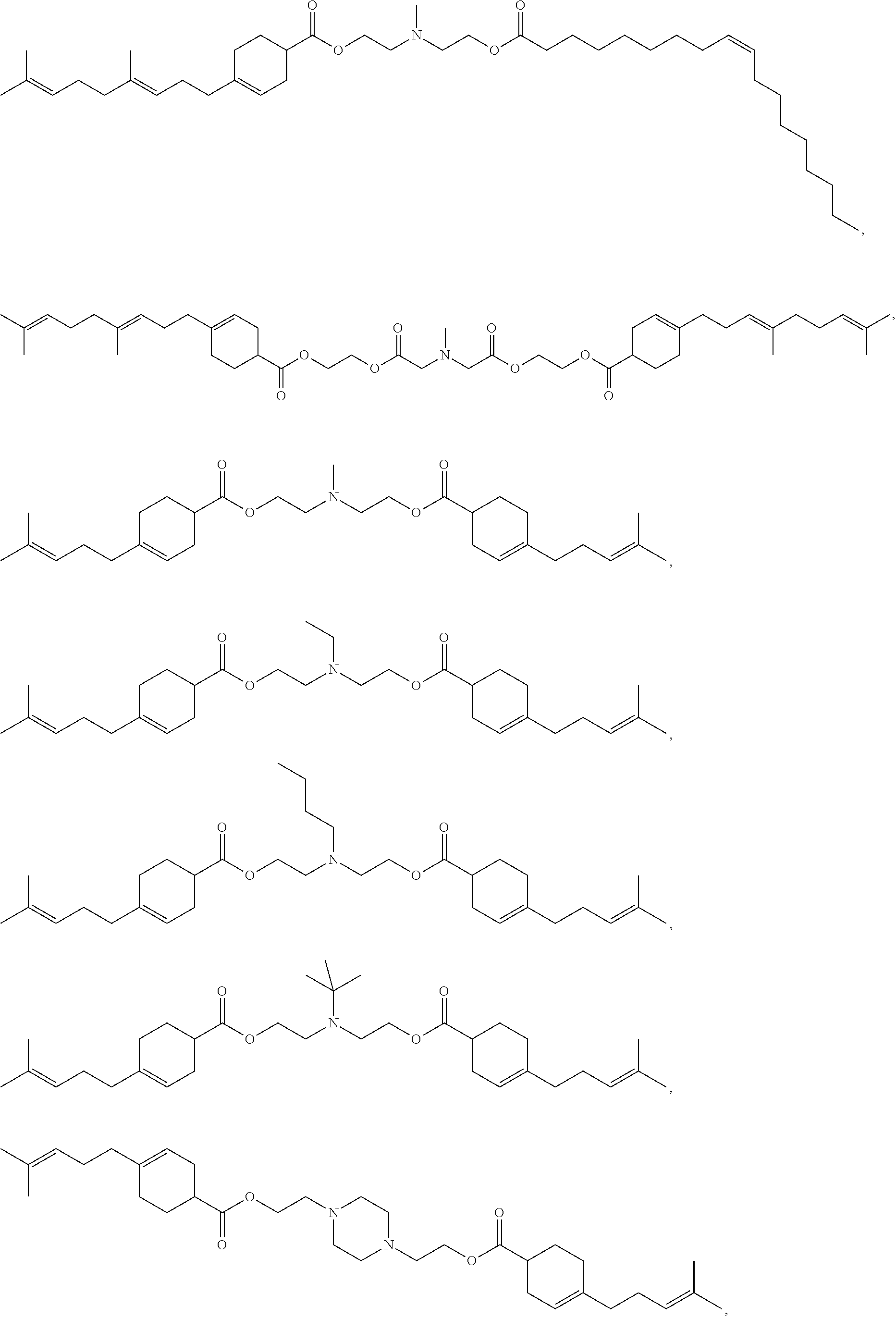

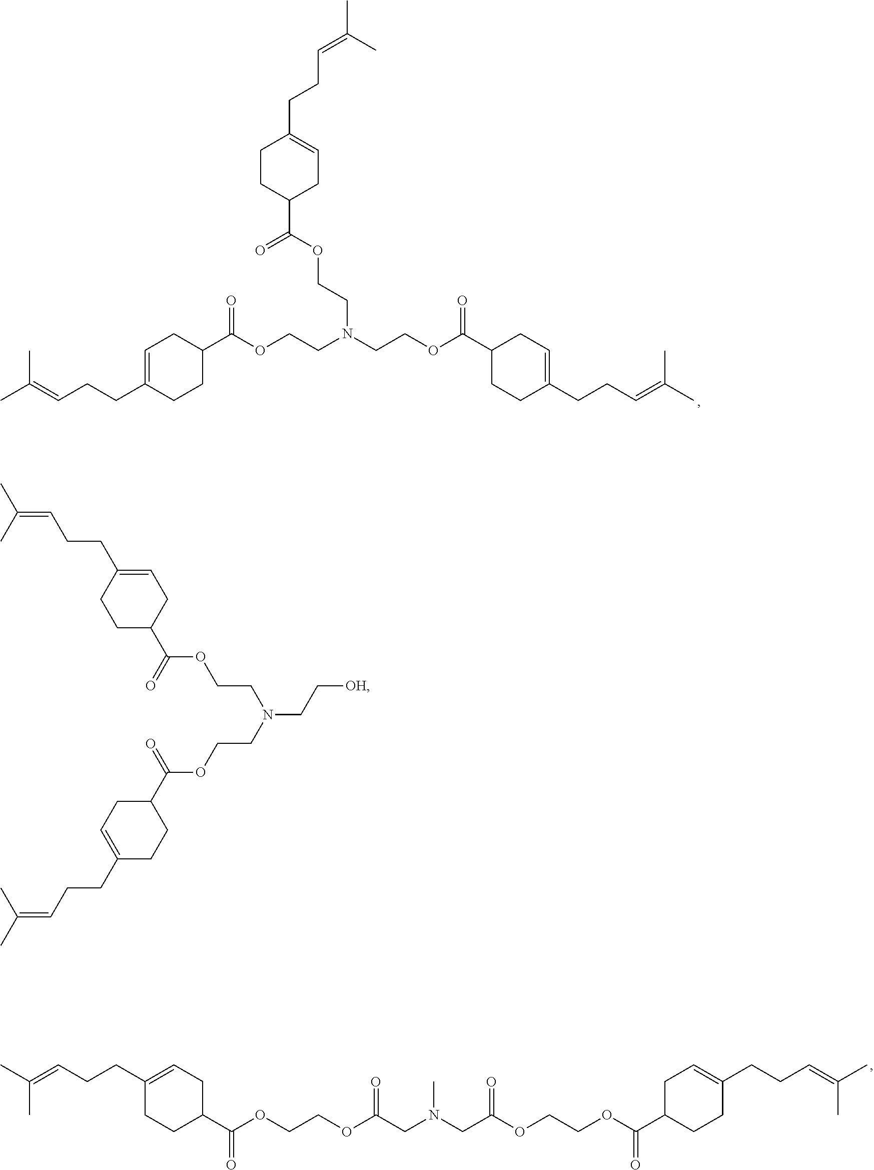

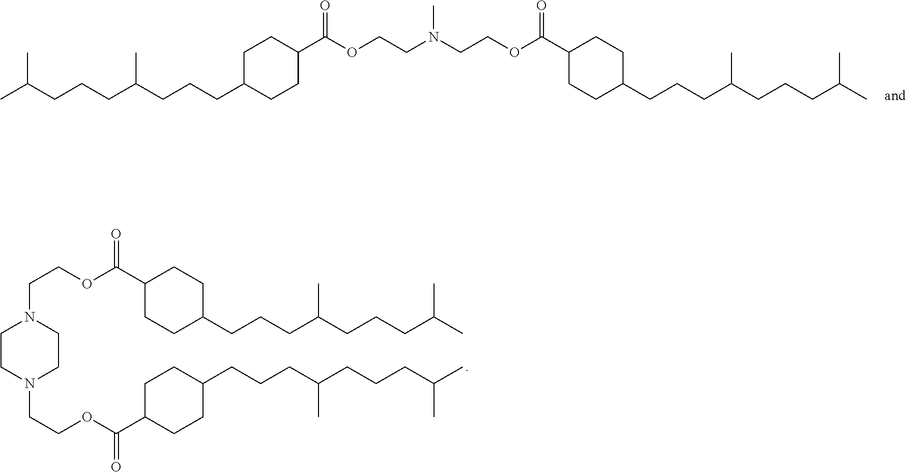 [SMILES: CC(C)CCCC(C)CCCC1CCC(C(=O)OCCN(C)CCOC(=O)C2CCC(CCCC(C)CCCC(C)C)CC2)CC1 ; CC(C)CCCC(C)CCCC1CCC(C(=O)OCCN2CCN(CCOC(=O)C3CCC(CCCC(C)CCCC(C)C)CC3)CC2)CC1]

In one aspect, the terpene-derived lipidoid compounds are represented by Formula (II):

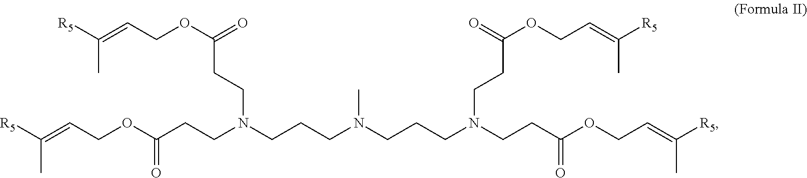

wherein each R₅ is

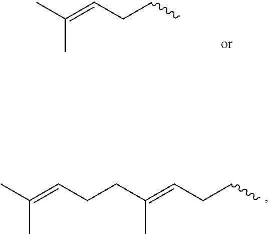

In some aspects, the terpene-derived cationic lipidoid compound of Formula (II) is a compound selected from the following:

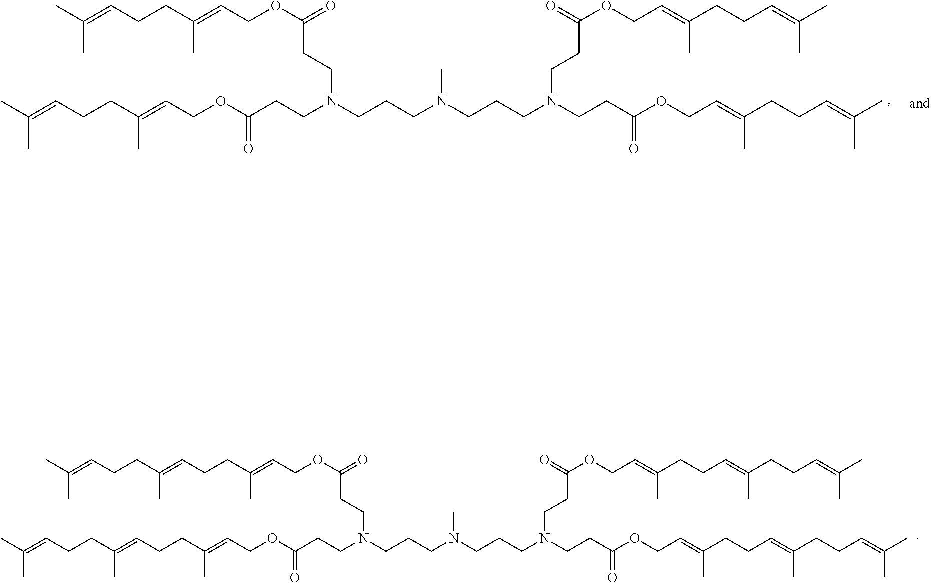

In one aspect, the terpene-derived lipidoid compounds are represented by Formula (III):

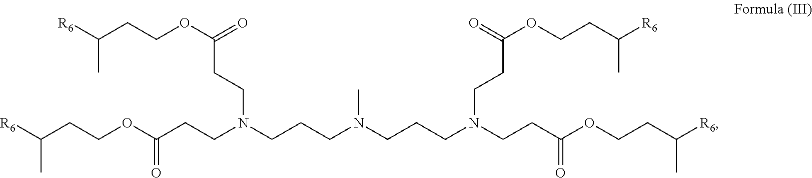

wherein each R₆ is

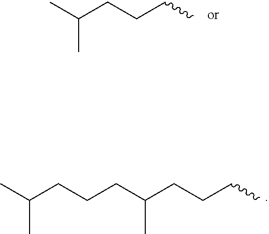

In some aspects, the terpene-derived cationic lipidoid compound of Formula (III) is a compound selected from the following:

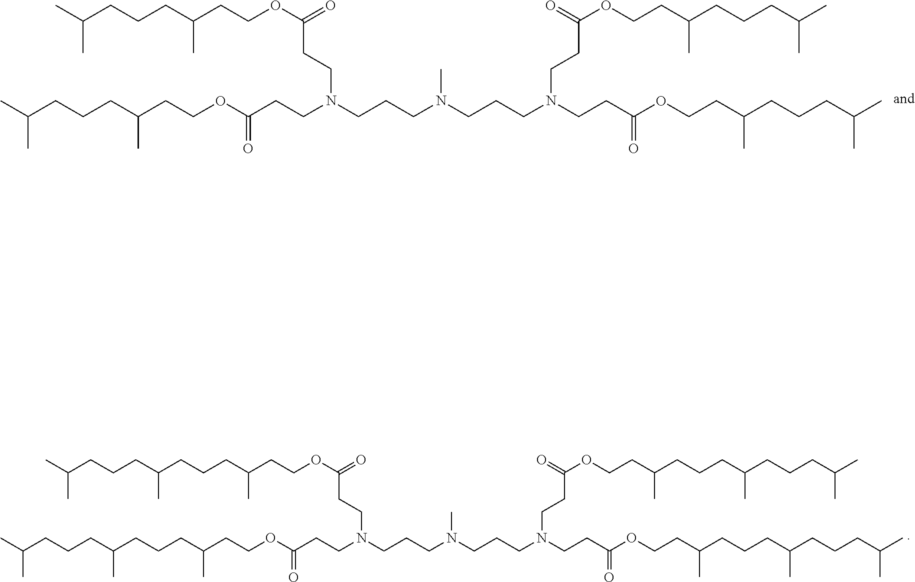

It will be understood that the compounds of any one of the Formulas disclosed herein and any pharmaceutically acceptable salts thereof, comprise stereoisomers, mixtures of stereoisomers, polymorphs of all isomeric forms of said compounds.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

It will be understood that in any of the formulae described herein, when a “—” is used to indicate linkage between two variables (e.g., A-B), the linkage could be one or more covalent bonds.

### General Methods for the Preparation of Compounds of Formula (I), Formula (II) or Formula (III) of the Present Disclosure

Compounds of Formula (I), Formula (II) or Formula (III) can be prepared using the reagents, intermediates, precursors, methods and schemes disclosed herein or using other commercially available reagents and methods known to those skilled in the art.

Some of the compounds of the invention, so called first generation lipidoids, can be prepared by the general schemes described below.

### Preparation of First Generation Lipidoids

In general, the first step in preparation of first generation cationic terpene-derived cationic compounds of Formula (I) of the present disclosure is the selection of an appropriate terpene, e.g., trans-beta-farnesene, beta-myrcene or other suitable biorenewable terpene, to prepare the multivalent “A” segment precursor. Mixtures of both 1,3-& 1,4-regioisomers are formed and the mixtures continued further without separation. For example:

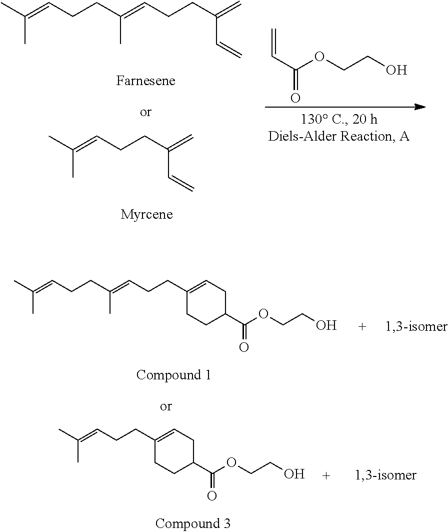

Suitable Diels-Alder products for use in the methods herein include:

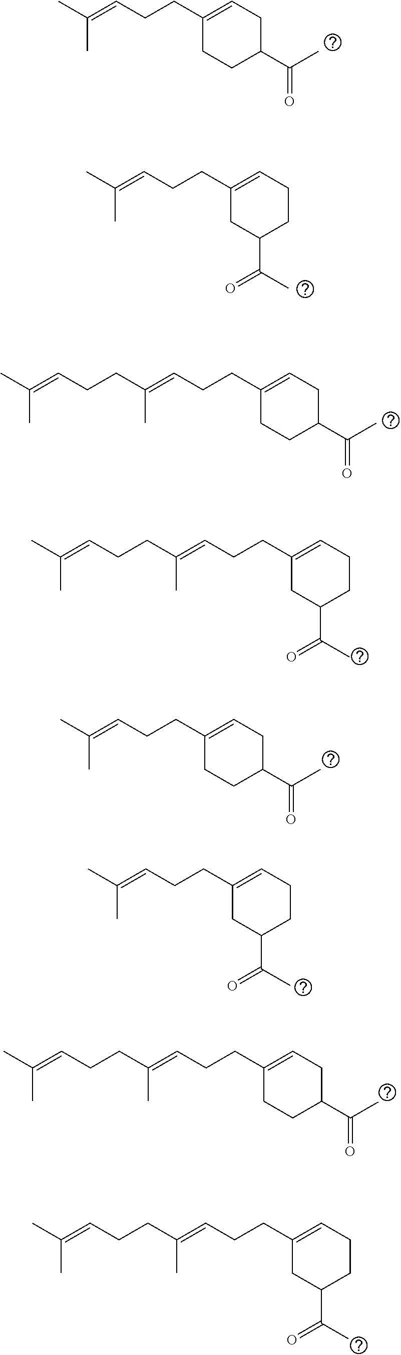

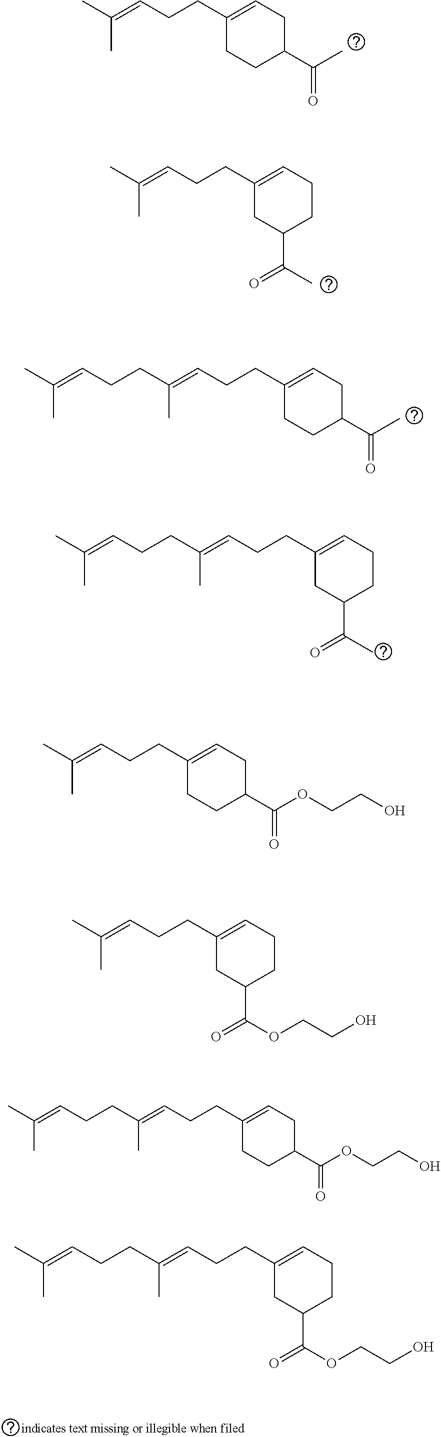

### General Procedure for Diels-Alder Reaction (A)

In a glass tube, trans-β-farnesene or myrcene (1.0 eq) and 2-hydroxyethyl acrylate (1.2 eq) were taken and the tube was sealed. The reaction was then stirred for 20 h at 130° C. The cooled reaction mixture was purified by silica gel flash chromatography (eluent: 20-30% EtOAc/hexane) to give compounds 1 or 3 as colorless oils.

Suitable temperatures are generally between 30° C. and 150° C. Higher temperatures are generally associated with higher yields.

Following the Diels-Alder Reaction, one optional step is hydrogenation which can be carried out as follows:

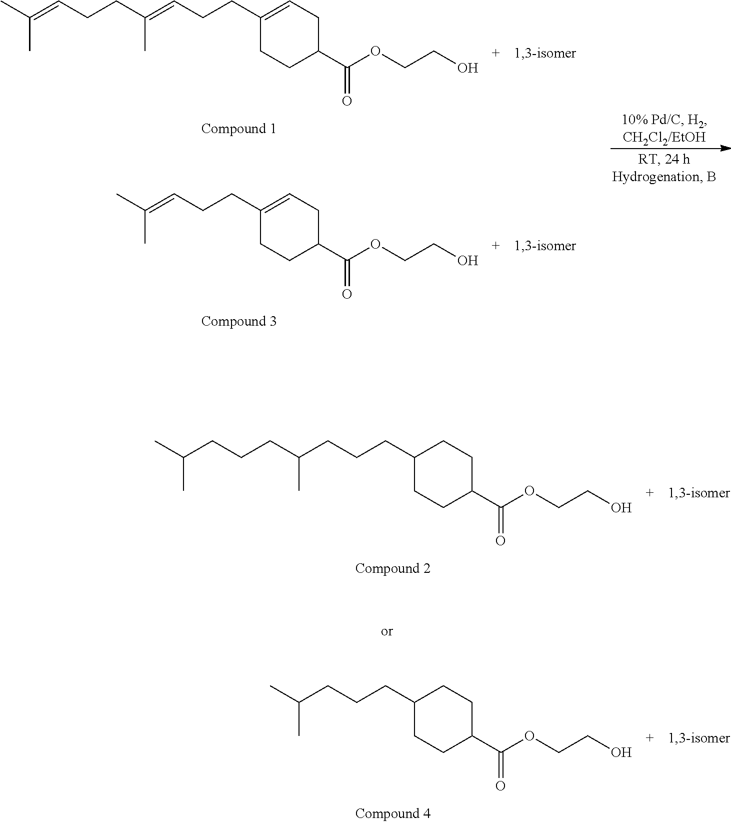 [SMILES: CC(C)=CCC/C(C)=C/CCC1=CCC(C(=O)OCCO)CC1 ; CC(C)=CCCC1=CCC(C(=O)OCCO)CC1 ; CC(C)CCCC(C)CCCC1CCC(C(=O)OCCO)CC1 ; CC(C)CCCC1CCC(C(=O)OCCO)CC1]

### General Procedure for Hydrogenation (B)

In a round-bottomed flask, compound 1 or 3 was dissolved in mixed solvents of ethanol and CH₂Cl₂ (4:1). To this solution, 10% Pd/C (10% w/w) was added in one portion and the resulting dark suspension was stirred under a hydrogen atmosphere for 24 h at room temperature. The reaction mixture was filtered through celite and rinsed with CH₂Cl₂ (3×). The filtrate was evaporated to give the hydrogenated compounds. The resulting residues proceeded to the next step without any purification.

Compounds which were prepared through either Diels-Alder reaction (A) or through hydrogenation (B) can then undergo acrylate installation (C) to provide myrcene acrylates (MA), farnesene acrylates (FA) (using products of Diels-Alder reaction) or hydrogenated myrcene acrylates (HMA) or hydrogenated farnesene acrylates (HFA) (using products of hydrogenation reaction):

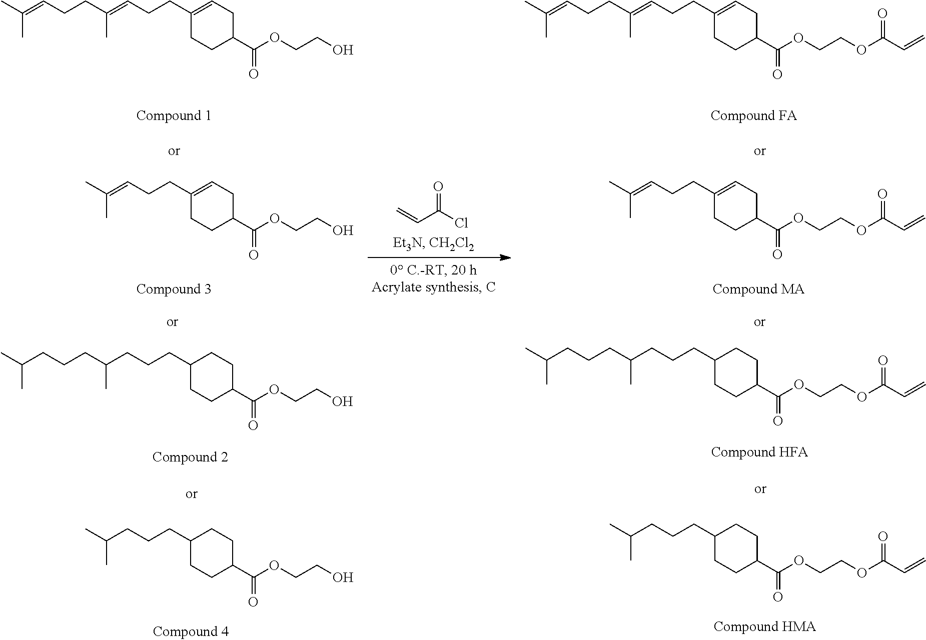

While the compounds in the scheme above are shown as 1,4 isomers, the compounds also include 1,3 isomers.

### General Procedure for Acrylate Synthesis (C)

In a round-bottomed flask, compound 1 or 2 or 3 or 4 (1.0 eq) was mixed with triethylamine (2.0 eq) in anhydrous CH₂Cl₂. The solution was cooled to 0° C. with an ice/water bath and acryloyl chloride (2.0 eq) was added dropwise over a period of 10 minutes. The resulting mixture was stirred for 20 h allowing the temperature to rise to room temperature. The reaction was quenched by adding a saturated aqueous solution of sodium bicarbonate (20 mL) followed by CH₂Cl₂ (20 mL). Both layers were separated and the aqueous layer was extracted with CH₂Cl₂ (3×). Combined organic extracts were washed with brine solution (20 mL); dried over sodium sulfate; and evaporated. The reaction crude was then purified using silica gel flash chromatography (eluent: 10-15% EtOAc/hexane).

Finally, first generation lipidoids can be prepared from these acrylate compounds through Aza-Michael Addition (D) as follows:

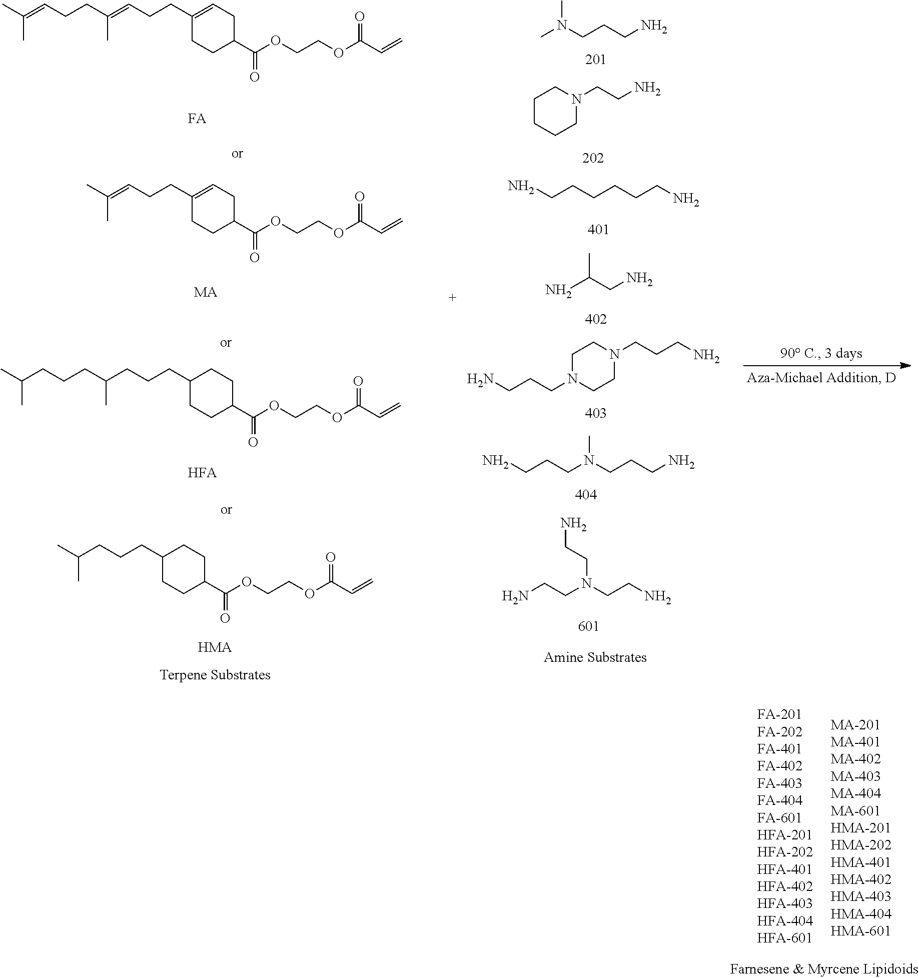

While the compounds in the scheme above are shown as 1,4 isomers, the compounds also include 1,3 isomers.

### General Procedure for Aza-Michael Addition (D)

In a 4-dram glass vial, acrylate (1.0 eq), and amine ([2n+1.0] eq; where n is the number of tails in the amine) were mixed and the vial was capped. The resulting reaction mixture was stirred for 3 days at 90° C. The reaction was cooled and purified by silica gel flash chromatography (eluent: 30-50% EtOAc/hexane or 0-10% MeOH/CH₂Cl₂).

Suitable temperatures are generally between 30° C. and 150° C.

### Further Derivatization of First Generation Lipidoids

Furthermore, the compounds of the invention can be functionalized further with alkyl chain thiols (dodecane thiol, and 3-methyl butyl thiol). In short, alkyl chains may be added to the unsaturation through thiol-ene reaction as follows.

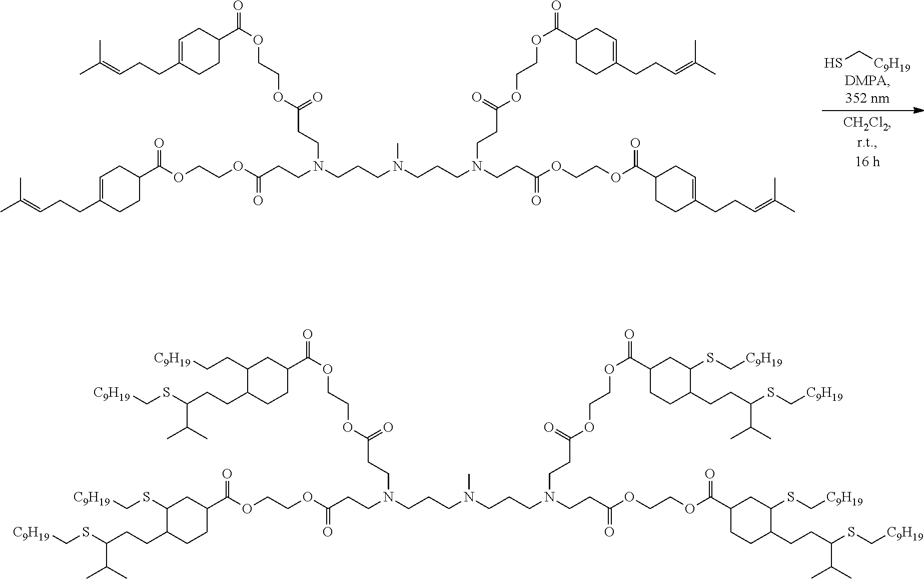

While the compounds in the scheme above are shown as 1,4 isomers, the compounds also include 1,3 isomers.

In a 4 mL quartz tube, MA-404 (50 mg, 1.0 eq), 1-dodecanethiol (63 mg, 10.0 eq), and DMPA (19 mg, 2.0 eq) were dissolved in anhydrous DCM (2.0 mL). The resulting solution was degassed with nitrogen for 5 minutes before irradiating with a UV light of wavelength 352 nm for 16 h at room temperature in a UV chamber. The tube was taken out of the UV chamber, reaction mixture was purified by silica gel column chromatography with 5% MeOH/DCM to afford 54 mg (53%) of MA-404-DT as a viscous oil. 1H NMR (400 MHz, Chloroform-d) δ 4.36-4.13 (m, 20H), 3.51-2.29 (m, 61H), 2.27-1.46 (m, 59H), 1.42-0.54 (m, 160H).

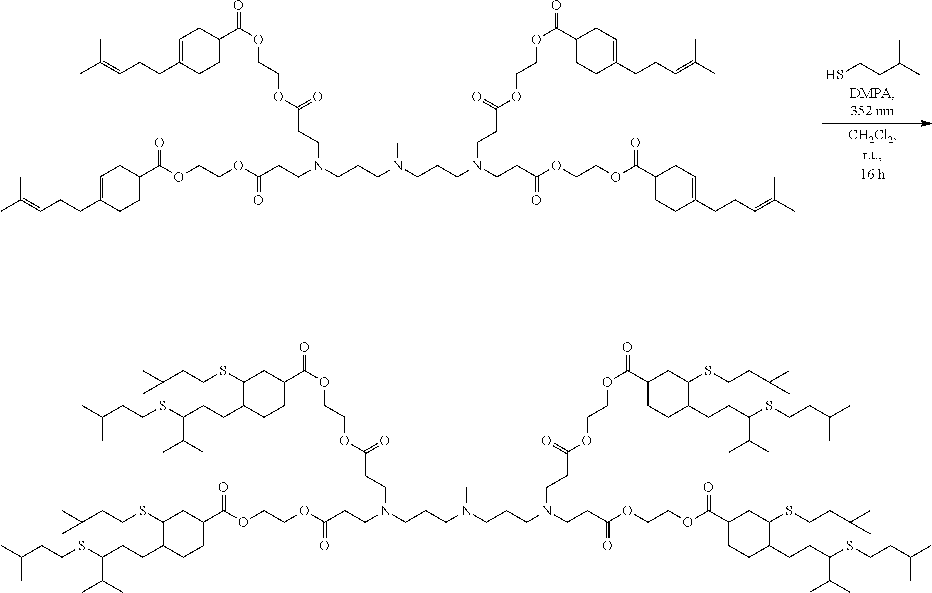

While the compounds in the scheme above are shown as 1,4 isomers, the compounds also include 1,3 isomers.

In a 4 mL quartz tube, MA-404 (50 mg, 1.0 eq), 3-methylbutyl thiol (38 mg, 10.0 eq), and DMPA (19 mg, 2.0 eq) were dissolved in anhydrous DCM (2.0 mL). The resulting solution was degassed with nitrogen for 5 minutes before irradiating with a UV light of wavelength 352 nm for 16 h at room temperature in a UV chamber. The tube was taken out of the UV chamber, reaction mixture was purified by silica gel column chromatography with 5% MeOH/DCM to afford 27 mg (34%) of MA-404-MBT as a viscous oil. ¹H NMR (400 MHz, Chloroform-d) δ 4.35-4.15 (m, 16H), 3.87-1.80 (m, 83H), 1.78-1.06 (m, 58H), 1.04-0.48 (m, 62H).

### Preparation of Second Generation Lipidoids

Second generation lipidoids of the invention (which also fall within the generic Formula I) can be prepared as follows.

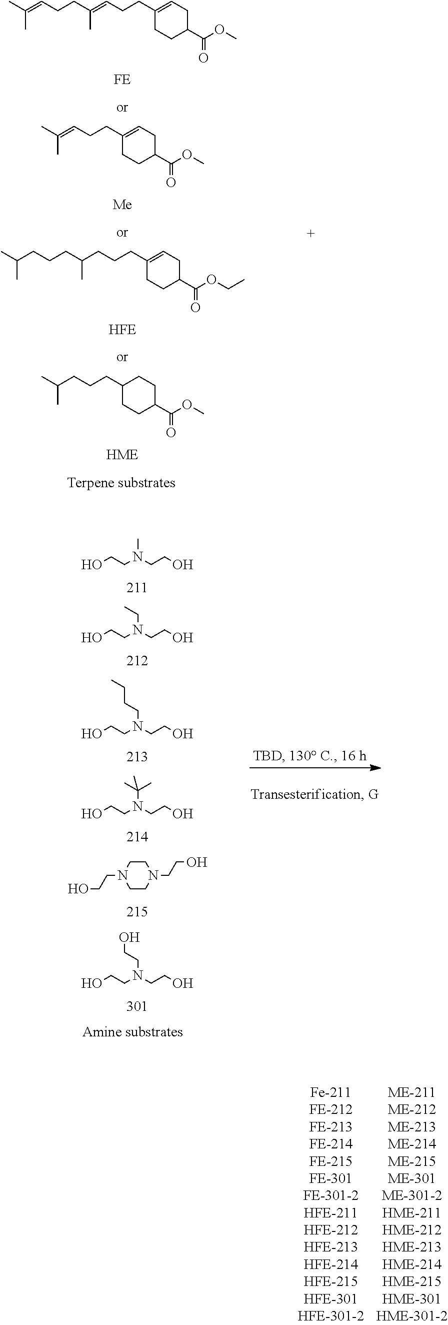

While the compounds in the scheme above are shown as 1,4 isomers, the compounds also include 1,3 isomers.

Structures of farnesene lipidoids that can be obtained by such scheme are as follows:

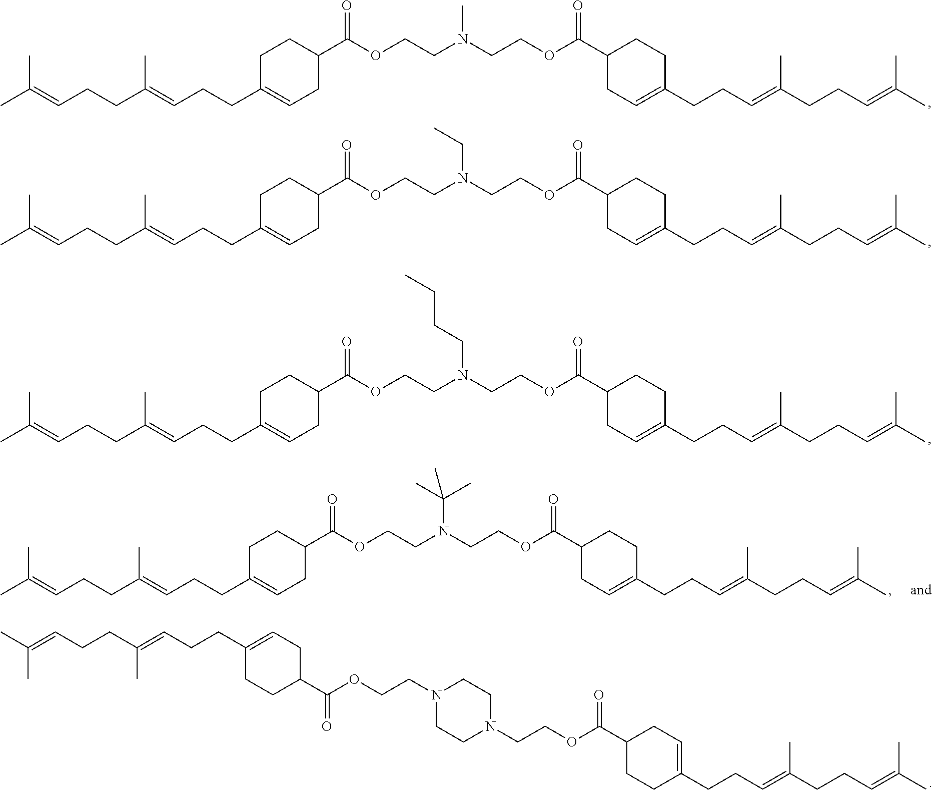

Structures of hybrid farnesene lipidoids that can be obtained by such scheme are as follows:

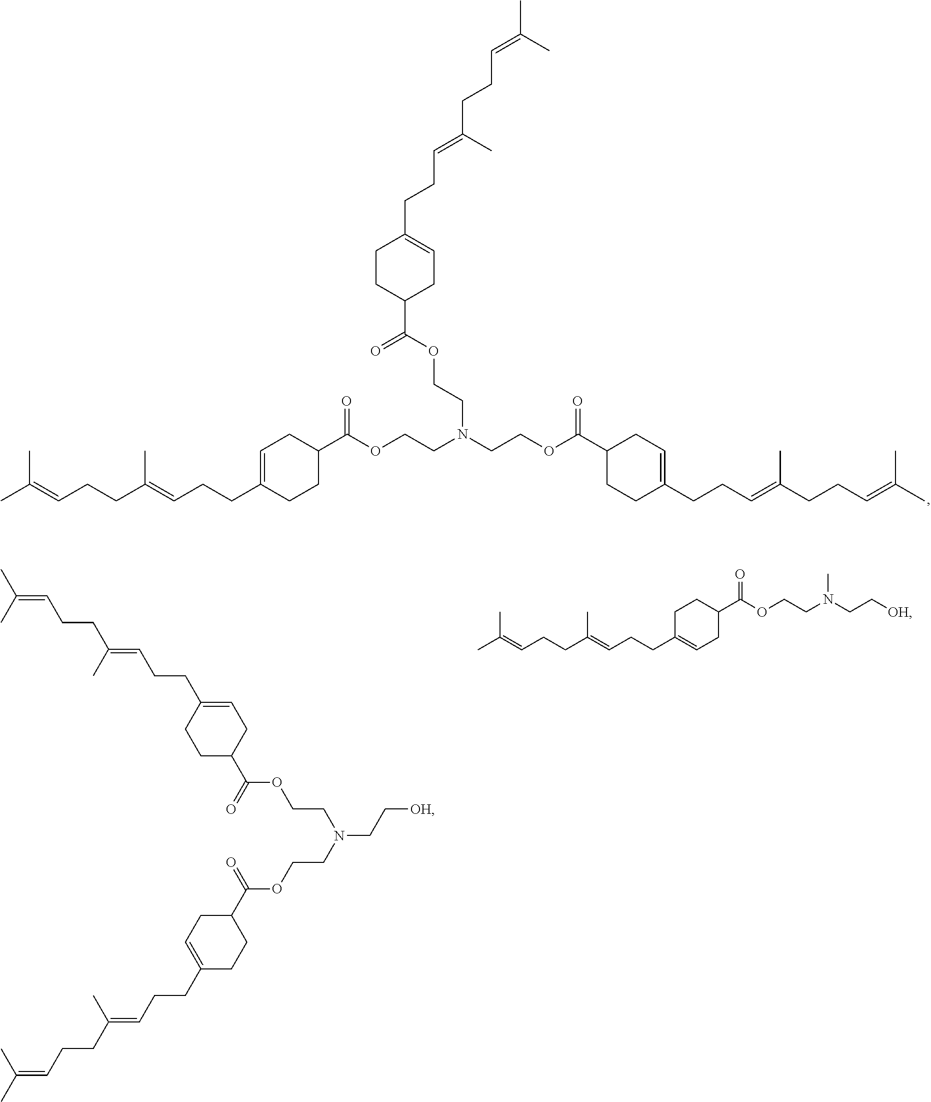

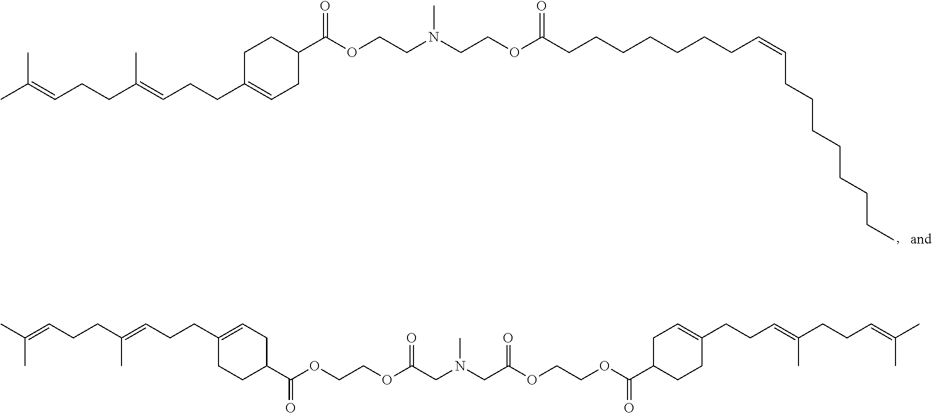

Structures of myrcene lipidoids that can be obtained by such scheme are as follows:

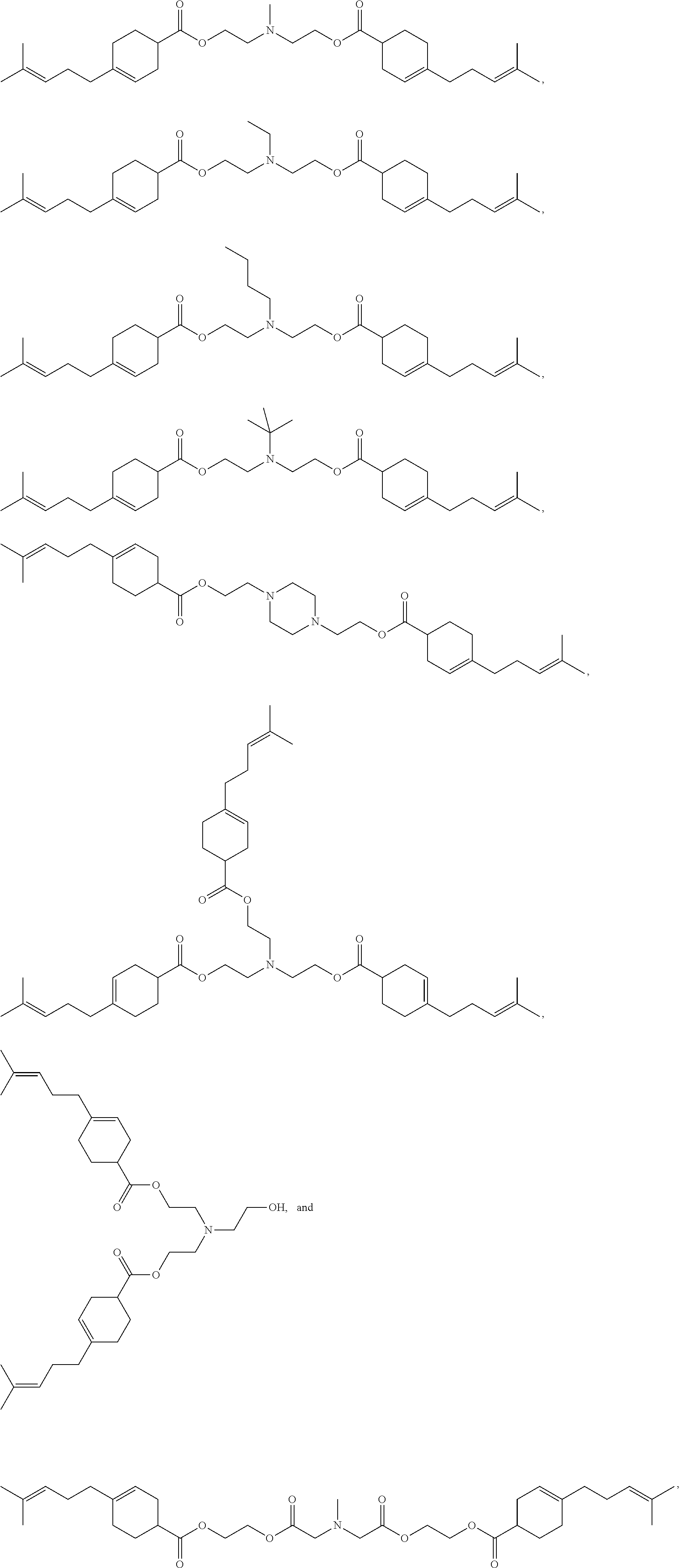

Reaction conditions for the synthesis of second generation lipidoids are generally as follows.

### General Procedure for Diels-Alder Reaction (E)

In a 4 mL sealed tube, farnesene or myrcene (1.0 eq) is combined with methyl acrylate (1.1 eq). The tube was sealed and the reaction was stirred for 20 h at 130° C. The cooled reaction mixture was purified by silica gel flash column chromatography with 3-5% EtOAc/hexane eluants. This reaction produces an unequal mixture of 1,3- and 1,4-regioisomers that cannot be separated using conventional column chromatographic methods. This mixture of regioisomers was used in subsequent reactions without isomer separation.

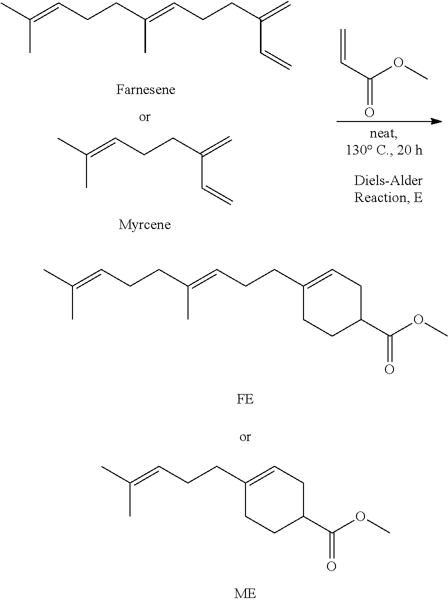

Suitable temperatures are generally between 30° C. and 150° C. Higher temperatures are generally associated with higher yields.

While the compounds in the scheme above are shown as 1,4 isomers, the compounds also include 1,3 isomers.

### General Procedure for Hydrogenation Reaction (F)

In a round-bottomed flask, compounds FE or ME was dissolved in mixed solvents of ethanol and CH₂Cl₂ (4:1). To this solution, 10% Pd/C (10% w/w) was added in one portion and the resulting dark suspension was stirred under a hydrogen atmosphere for 20 h at room temperature. The reaction mixture was filtered through celite and rinsed with CH₂Cl₂ (3×). The filtrate was evaporated to give the hydrogenated compounds. The resulting residues proceeded to the next step without any purification.

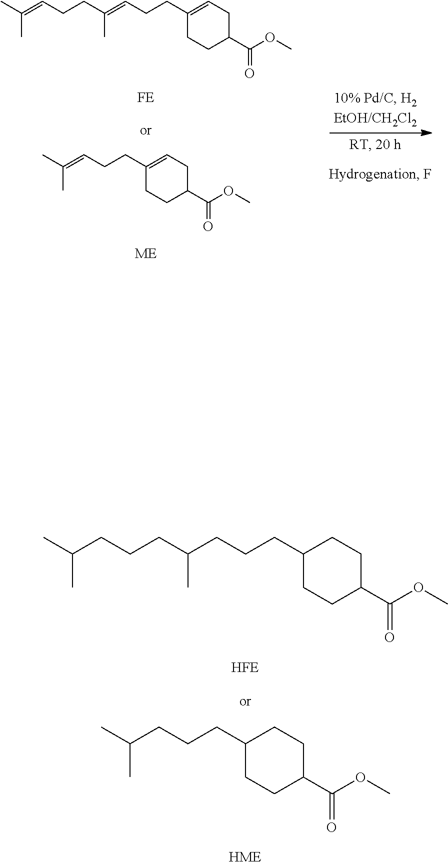

While the compounds in the scheme above are shown as 1,4 isomers, the compounds also include 1,3 isomers.

### General Procedure for Transesterification Reaction (G)

In a 4 mL glass tube, FE or ME (2.5 eq) is combined with an appropriate amine (1.0 eq) and triazabicyclodecene (TBD) (0.5 eq). The tube was sealed and the reaction mixture was stirred for 16 h at 130° C. The cooled reaction mixture was purified by silica gel flash column chromatography with 10-50% EtOAc/hexane or 5-10% MeOH/DCM eluants.

### General Procedure for Synthesis of FE-216 and ME-216 Lipidoids (H)

In a 20 mL scintillation glass vial, methyliminodiacetic acid (216) (1.0 eq), 1-ethyl-3-(3-dimethylamino propyl)carbodiimide (EDC-HCl) (2.2 eq), and dimethyl aminopyridine (DMAP) (2.2 eq) were dissolved in dichloromethane. The mixture was stirred for 15 minutes at room temperature before adding Compound 1 or Compound 3 (2.2 eq) dissolved in dichloromethane. The reaction was then stirred for 20 h at room temperature. The reaction mixture was transferred to a separatory funnel, added 20 mL of distilled water, and extracted with dichloromethane (3×). Combined organic extracts were dried over Na₂SO₄, filtered to a round bottom flask and the solvent was evaporated under reduced pressure. The crude residue was purified by silica gel flash column chromatography with 20% EtOAc/hexane eluant.

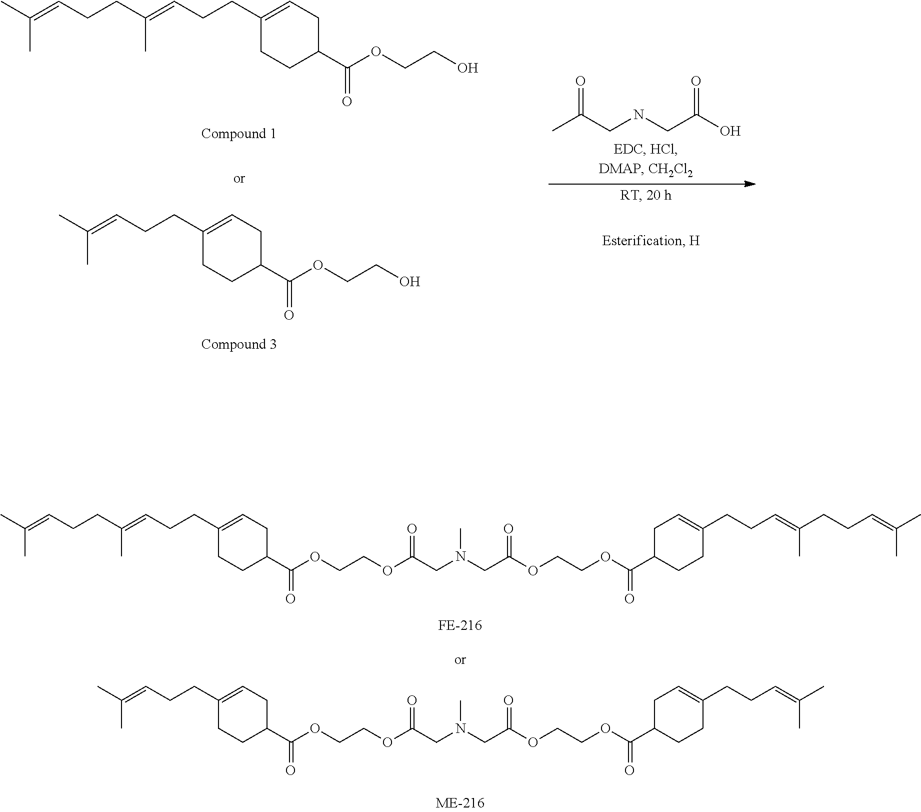

### General Procedure for Mono-transesterification Reaction (I)

In a 4 mL glass tube, FE or ME (1.1 eq) is combined with an appropriate amine (1.0 eq). The tube was sealed and the reaction mixture was stirred for 20 h at 130° C. The cooled reaction mixture was purified by silica gel flash column chromatography with 5-10% MeOH/DCM eluants.

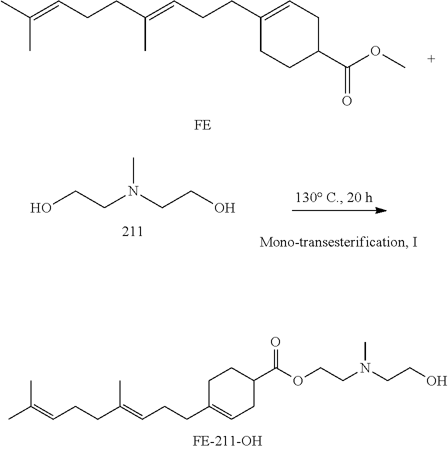

### General Procedure for Synthesis of Unsymmetrical Lipidoids (J)

In a 20 ml, scintillation glass vial, appropriate carboxylic acid (1.0 eq), 1-ethyl-3-(3-dimethyl aminopropyl)carbodiimide (EDC-HCl) (1.1 eq), and dimethyl aminopyridine (DMAP) (1.1 eq) were dissolved in dichloromethane. The mixture was stirred for 15 minutes at room temperature before adding monoester (1.1 eq) dissolved in dichloromethane. The reaction was then stirred for 20 h at room temperature. The reaction mixture was transferred to a separatory funnel, added 20 mL of distilled water, and extracted with dichloromethane (3×). Combined organic extracts were dried over Na₂SO₄, filtered to a round bottom flask and the solvent was evaporated under reduced pressure.

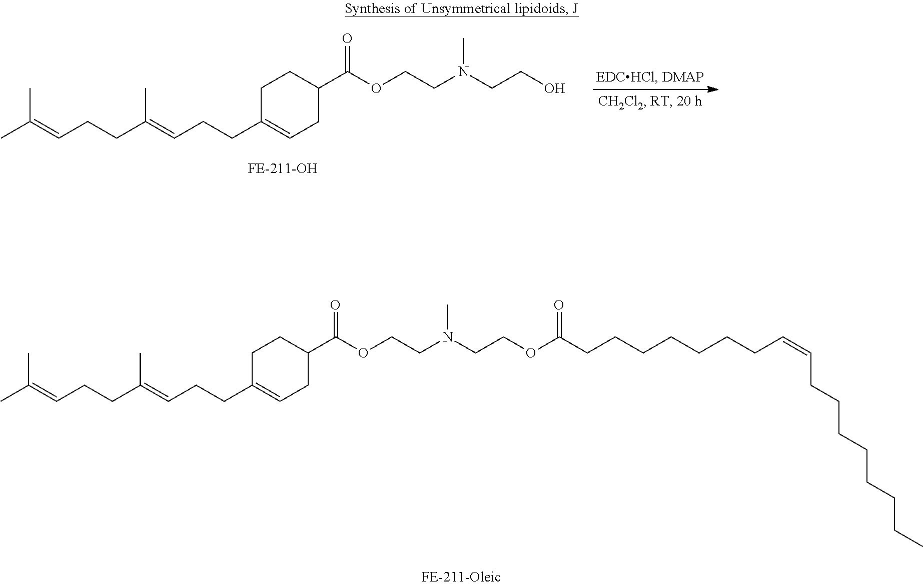 [SMILES: CC(C)=CCC/C(C)=C/CCC1=CCC(C(=O)OCCN(C)CCO)CC1 ; CCCCCCCC/C=C\CCCCCCCC(=O)OCCN(C)CCOC(=O)C1CC=C(CC/C=C(\C)CCC=C(C)C)CC1]

### Alternative General Procedure for Synthesis of Lipidoids (K)

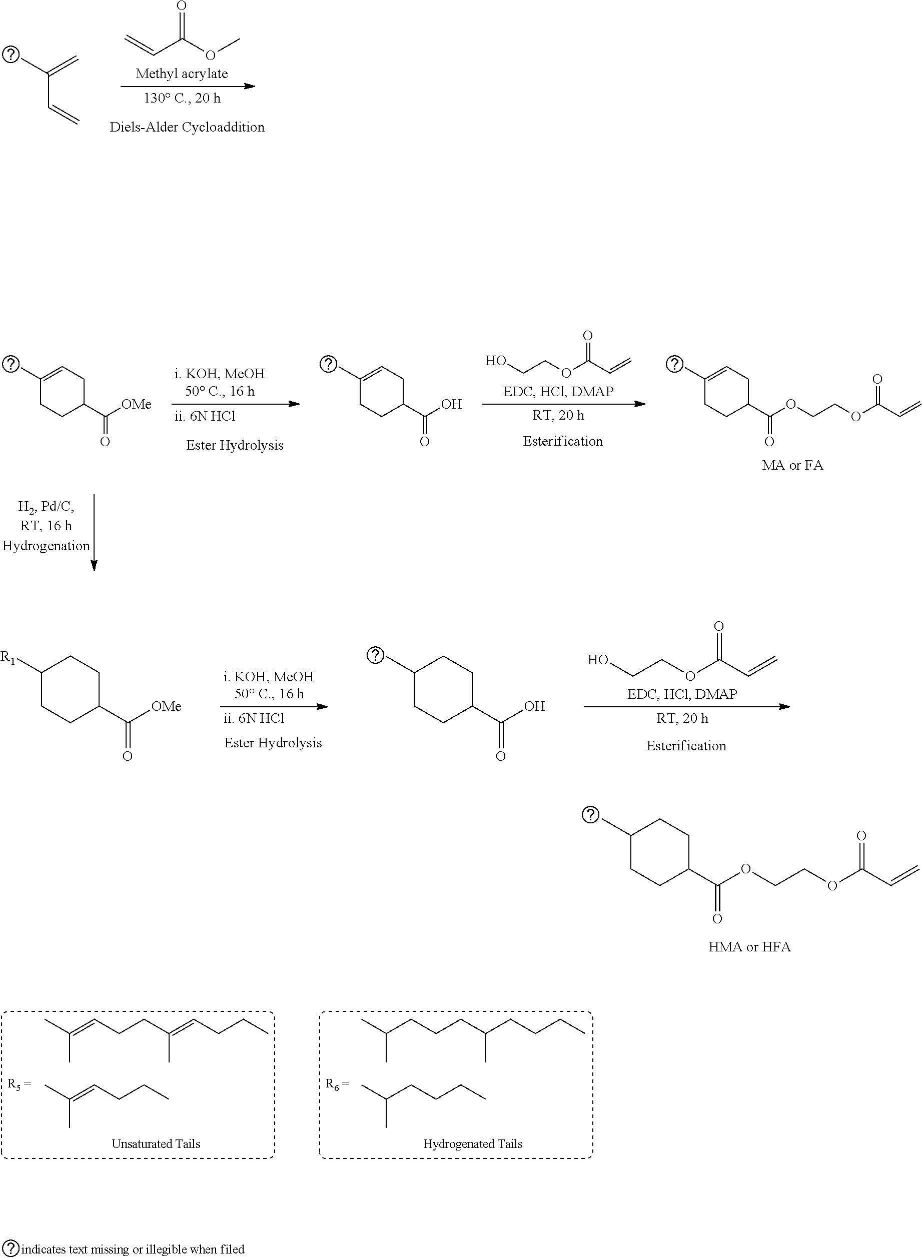

### Synthesis of MA

### Step-1—Diels-Alder Cycloaddition:

Myrcene (7.2 g, 52.8 mmol, 1.0 eq) and methyl acrylate (5.0 g, 58.1 mmol, 1.1 eq) were taken in a sealed tube. The tube was then sealed and stirred for 16 h at 130° C. The cooled reaction mixture was transferred to a round-bottom flask with CH₂Cl₂ and evaporated. The crude residue was purified by silica gel flash column chromatography with 3% EtOAc/hexane as eluants. TLC: 5% EtOAc/hexane, Iodine stain. 9.1 g (77%), colorless oil. ¹H NMR (400 MHz, Chloroform-d) δ 5.42-5.35 (m, 1H), 5.12-5.04 (m, 1H), 3.69-3.66 (m, 3H), 2.60-2.43 (m, 1H), 2.30-1.86 (m, 10H), 1.67 (s, 3H), 1.59 (s, 3H).

Suitable temperatures are generally between 30° C. and 150° C. Higher temperatures are generally associated with higher yields.

Step-2—Ester hydrolysis:

In a 20 mL scintillation vial, the myrcene Diels-Alder adduct (634 mg, 1.0 eq) was dissolved in methanol (10 mL) and was added potassium hydroxide (239 mg, 1.5 eq). The reaction mixture was stirred for 16 h at 50° C. Water (50 mL) was added to the cooled reaction and extracted with diethyl ether (3×50 mL). The aqueous layer was acidified with 6N HCl until pH 2 and extracted with ethyl acetate (4×50 mL). The combined ethyl acetate extracts were washed with brine solution (50 mL); dried over Na₂SO₄; filtered; and evaporated. The resulting residue proceeded to the subsequent reaction without any purification. 561 mg (94%), colorless oil. ¹H NMR (500 MHz, Chloroform-d) δ 5.45-5.37 (m, 1H), 5.14-5.05 (m, 1H), 2.66-2.50 (m, 1H), 2.36-1.93 (m, 10H), 1.69 (s, 3H), 1.61 (s, 3H).

This reaction can be achieved at lower temperatures with longer times for better yields.

### Step-3—Esterification:

In a 20 mL scintillation vial, the myrcene-based carboxylic acid (530 mg, 1.0 eq) from the above reaction was dissolved in CH₂Cl₂ (5 mL). To the solution, N-(3-dimethylaminopropyl)-N′-ethylcarbodiimide hydrochloride (585 mg, 1.2 eq) and 4-dimethylaminopyridine (124 mg, 0.4 eq) were added. The reaction mixture was then stirred for 15 mins at room temperature. To this solution was added 2-hydroxyethyl acrylate (0.35 mL, 1.2 eq) dropwise and the resulting solution was stirred for 20 h at room temperature. Water (50 mL) was added and extracted with CH₂Cl₂ (4×50 mL). The combined organic extracts were washed with brine (50 mL); dried over Na₂SO₄; filtered; and evaporated. The crude product was purified by silica gel flash column chromatography with 5-6% EtOAc/hexane as eluants to give the title product MA as a colorless oil. 498 mg (64%), colorless oil. ¹H NMR (400 MHz, Chloroform-d) δ 6.42 (dt, J=17.3, 1.2 Hz, 1H), 6.12 (ddd, J=17.4, 10.4, 1.0 Hz, 1H), 5.85 (dd, J=10.4, 1.5 Hz, 1H), 5.44-5.31 (m, 1H), 5.14-4.99 (m, 1H), 4.42-4.27 (m, 4H), 2.66-2.42 (m, 1H), 2.28-1.87 (m, 10H), 1.66 (s, 3H), 1.58 (s, 3H).

### Synthesis of HMA

### Step-2—Hydrogenation:

In a 100 mL round bottom flask, the myrcene Diels-Alder adduct (3.86 g) was dissolved in mixed solvents of CH₂Cl₂ and ethanol (30 mL, 1:5 v/v). To this solution, 10% Pd/C (512 mg, 15% w/w to Diels-Alder adduct) was added in one portion and the resulting dark suspension was stirred for 16 h at room temperature under a hydrogen atmosphere. The hydrogen balloon was taken off and the reaction mixture was filtered through celite and rinsed with CH₂Cl₂ (5×50 mL). The filtrate was evaporated to dryness. The compound obtained was proceeded to the next reactions without any purification. TLC: 5% EtOAc/hexane, Iodine stain (disappearance of starting material). 3.76 g (quant.), colorless oil. ¹H NMR (400 MHz, Chloroform-d) δ 3.67-3.64 (m, 3H), 2.55-2.15 (m, 1H), 2.03-1.03 (m, 15H), 0.95-0.81 (m, 7H).

### Step-3—Ester Hydrolysis:

In a 100 mL round bottom flask, the myrcene-based hydrogenated acid (6.5 g, 1.0 eq) was dissolved in methanol (40 mL) and was added potassium hydroxide (2.4 g, 1.5 eq). The reaction mixture was stirred for 16 h at 50° C. Water (100 mL) was added to the cooled reaction and extracted with diethyl ether (3×50 mL). The aqueous layer was acidified with 6N HCl until pH 2 and extracted with ethyl acetate (4×100 mL). The combined ethyl acetate extracts were washed with brine solution (50 mL); dried over Na₂SO₄; filtered; and evaporated. The resulting residue proceeded to the subsequent reaction without any purification. 5.2 g, colorless oil. ¹H NMR (500 MHz, Chloroform-d) δ 2.61-2.19 (m, 1H), 2.10-1.63 (m, 4H), 1.62-1.00 (m, 12H), 0.98-0.81 (m, 7H).

This reaction can be achieved at lower temperatures with longer times for better yields.

### Step-4—Esterification:

In a 100 mL round bottom flask, the myrcene-based carboxylic acid (5.16 g, 1.0 eq) from the above reaction was dissolved in CH₂Cl₂ (50 mL). To the solution, N-(3-dimethylaminopropyl)-N′-ethylcarbodiimide hydrochloride (5.6 g, 1.2 eq) and 4-dimethylaminopyridine (1.2 g, 0.4 eq) were added. The reaction mixture was then stirred for 15 mins at room temperature. To this solution was added 2-hydroxyethyl acrylate (3.35 mL, 1.2 eq) dropwise and the resulting solution was stirred for 20 h at room temperature. Water (50 mL) was added and extracted with CH₂Cl₂ (4×100 mL). The combined organic extracts were washed with brine (50 mL); dried over Na₂SO₄; filtered; and evaporated. The crude product was purified by silica gel flash column chromatography with 5-6% EtOAc/hexane as eluants to give the title product HMA as a colorless oil. 5.7 g (76%), colorless oil. ¹H NMR (500 MHz, Chloroform-d) δ 6.51-6.33 (m, 1H), 6.22-6.05 (m, 1H), 5.92-5.80 (m, 1H), 4.43-4.24 (m, 4H), 2.75-2.16 (m, 1H), 2.02-1.64 (m, 4H), 1.59-0.98 (m, 12H), 0.89-0.84 (m, 6H).

### Alternative General Procedure for Synthesis of Lipidoids (L)

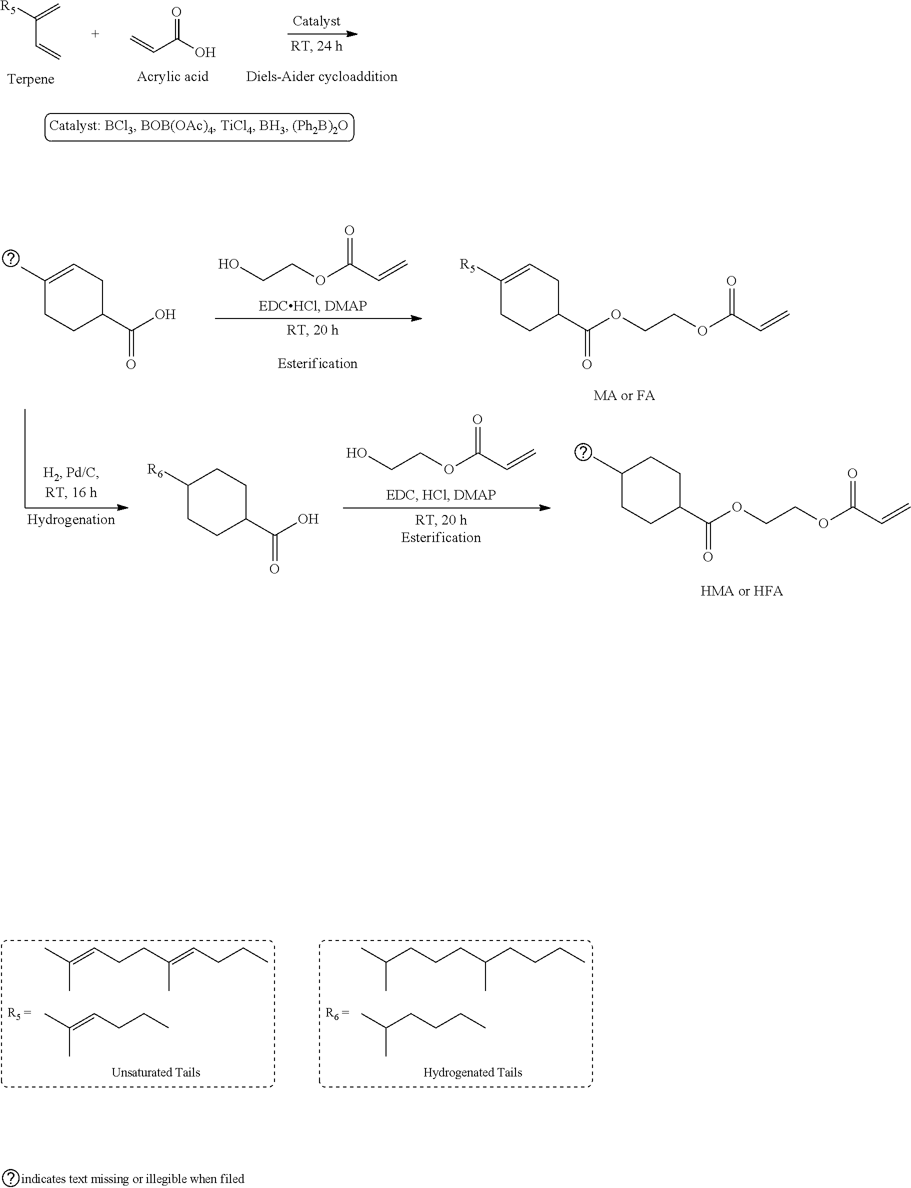

### Synthesis of HMA

### Diels-Alder Cycloaddition:

In a 4 mL sealed tube, acrylic acid (0.5 g, 1.0 eq), catalyst tetraacetyl diborate (BOB(OAc)₄) (40 mg, 2 mol %), and myrcene (1.04 g, 1.1 eq) were taken. The tube was then sealed and stirred for 3 days at room temperature. The reaction was then transferred to a separatory funnel with CH₂Cl₂ (50 mL) and added water (20 mL) followed by brine (20 mL). The separatory funnel was shaken and the organic layer was separated. The aqueous layer was extracted with CH₂Cl₂ (3×50 mL). Combined organic layers were dried over Na₂SO₄, filtered, and evaporated. The crude residue was adsorbed onto silica gel and purified through silica gel flash column chromatography with 10% EtOAc/hexane. 583 mg (40%), colorless semi-solid. ¹H NMR (500 MHz, Chloroform-d) δ 5.45-5.37 (m, 1H), 5.14-5.05 (m, 1H), 2.66-2.50 (m, 1H), 2.36-1.93 (m, 10H), 1.69 (s, 3H), 1.61 (s, 3H).

Suitable temperatures are generally between 30° C. and 150° C. Higher temperatures are generally associated with higher yields.

### Hydrogenation:

In a 100 mL round bottom flask, the myrcene-based Diels-Alder adduct (440 mg) was dissolved in mixed solvents of CH₂Cl₂ and ethanol (30 mL, 1:5 v/v). To this solution, 10% Pd/C (98 mg, 15% w/w to Diels-Alder adduct) was added in one portion and the resulting dark suspension was stirred for 16 h at room temperature under a hydrogen atmosphere. The hydrogen balloon was taken off and the reaction mixture was filtered through celite and rinsed with CH₂Cl₂ (5×50 mL). The filtrate was evaporated to dryness. The compound obtained was proceeded to the next reactions without any purification. TLC: 5% EtOAc/hexane, Iodine stain (disappearance of starting material). 425 mg (quant.), colorless oil. ¹H NMR (500 MHz, Chloroform-d) δ 2.61-2.19 (m, 1H), 2.10-1.63 (m, 4H), 1.62-1.00 (m, 12H), 0.98-0.81 (m, 7H).

### Esterification:

In a 20 mL scintillation vial, the hydrogenated myrcene-based carboxylic acid (425 mg, 1.0 eq) from the above reaction was dissolved in CH₂Cl₂ (5 mL). To the solution, N-(3-dimethylaminopropyl)-N′-ethylcarbodiimide hydrochloride (461 mg, 1.2 eq) and 4-dimethylaminopyridine (98 mg, 0.4 eq) were added. The reaction mixture was then stirred for 15 mins at room temperature. To this solution was added 2-hydroxyethyl acrylate (0.28 mL, 1.2 eq) dropwise and the resulting solution was stirred for 20 h at room temperature. Water (50 mL) was added and extracted with CH₂Cl₂ (4×50 mL). The combined organic extracts were washed with brine (50 mL); dried over Na₂SO₄; filtered; and evaporated. The crude product was purified by silica gel flash column chromatography with 5-6% EtOAc/hexane as eluants to give the title product HMA as a colorless oil. 538 mg (86%), colorless oil. ¹H NMR (500 MHz, Chloroform-d) S 6.51-6.33 (m, 1H), 6.22-6.05 (m, 1H), 5.92-5.80 (m, 1H), 4.43-4.24 (m, 4H), 2.75-2.16 (m, 1H), 2.02-1.64 (m, 4H), 1.59-0.98 (m, 12H), 0.89-0.84 (m, 6H).

### Alternative General Procedure for Synthesis of Lipidoids (M)

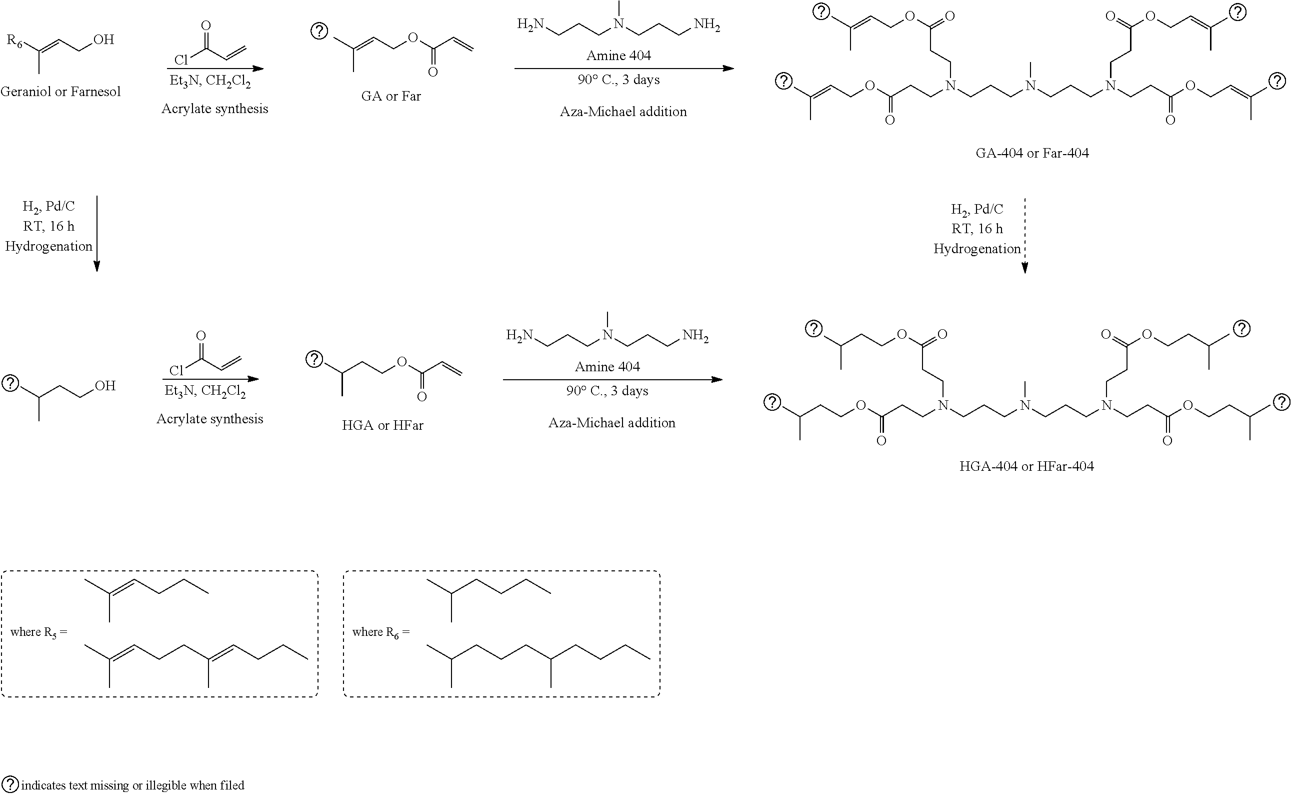

### Synthesis of GA-404

### Acrylate Synthesis:

In a 100 mL round bottom flask, geraniol (1.0 eq) was dissolved in anhydrous CH₂Cl₂ and was added triethylamine (1.5 eq). The reaction solution was cooled to 0° C. with ice/water bath. Acryloyl chloride (1.2 eq) was added to the reaction dropwise for 15 mins and the resulting pale-yellow solution was stirred for 16 h allowing the temperature to raise to room temperature (25° C.). The solvent in the reaction was evaporated and purified by silica gel flash column chromatography with 4-5% EtOAc/hexane as eluants. Colorless oil. ¹H NMR (400 MHz, Chloroform-d) δ 6.40 (dd, J=17.3, 1.5 Hz, 1H), 6.13 (dd, J=17.3, 10.4 Hz, 1H), 5.81 (dd, J=10.4, 1.5 Hz, 1H), 5.37 (tq, J=7.1, 1.3 Hz, 1H), 5.12-5.04 (m, 1H), 4.68 (dd, J=7.2, 0.9 Hz, 2H), 2.15-2.01 (m, 4H), 1.72 (s, 3H), 1.69-1.66 (m, 3H), 1.60 (s, 3H).

### Aza-Michael Addition:

In a 4 mL scintillation vial, the amine-404 (1.0 eq) and acrylate (4.5 eq) from the above reaction was taken. The capped vial was then stirred for 3 days at 90° C. The cooled reaction mixture was purified by silica gel flash column chromatography with 4-5% MeOH/CH₂Cl₂ as eluants. Pale-yellow oil. ¹H NMR (400 MHz, Chloroform-d) δ 5.31 (tq, J=7.0, 1.4 Hz, 411), 5.06 (tp, J=6.8, 1.5 Hz, 4H), 4.56 (d, J=7.1 Hz, 8H), 2.73 (t, J=7.1 Hz, 8H), 2.50-2.36 (m, 15H), 2.19-1.90 (m, 18H), 1.87-1.70 (m, 6H), 1.70-1.67 (m, 12H), 1.66 (s, 12H), 1.58 (s, 12H).

Suitable temperatures are generally between 30° C. and 150° C.

### Alternative General Procedure for Synthesis of Lipidoids (N)

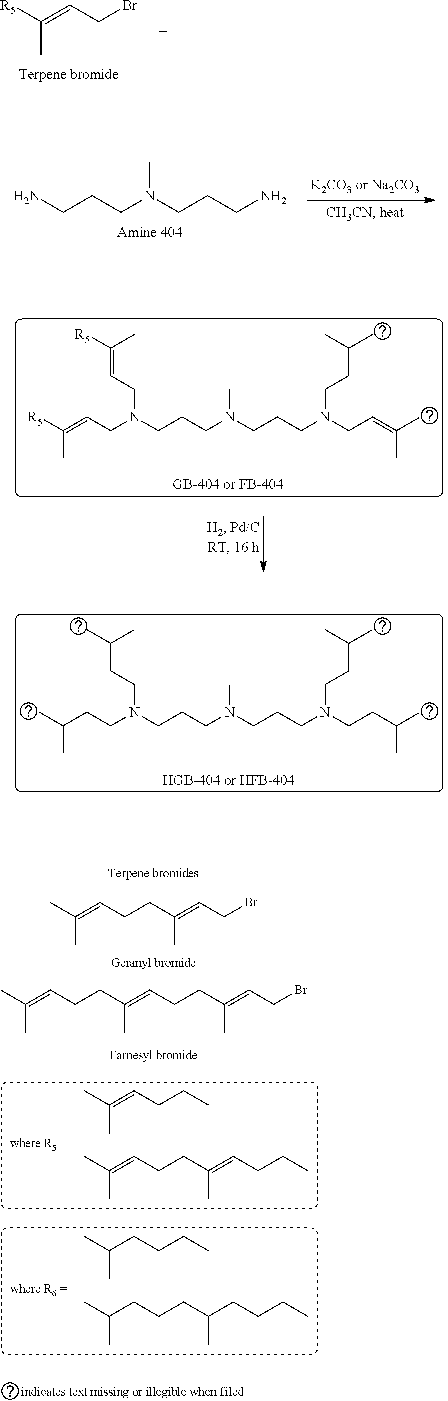

For all of the schemes listed above, similar solvents can be used instead of the solvents depicted in the schemes.

Further, all hydrogenated lipids (HMA-series, HFA-series, HGA-series, and HFar-series) can be prepared from their unsaturated analogs (MA-series, FA-series, GA-series, and Far-series) by direct hydrogenation with higher catalyst loading and longer times. HMA-404, for example, can be synthesized directly from MA-404 using any catalytic hydrogenation method (including the hydrogenation protocol given in the application with reaction conditions modified).

The compounds of Formula (I), Formula (II), and Formula (III) can be incorporated into pharmaceutical compositions.

### Pharmaceutical Compositions

The present disclosure provides pharmaceutical compositions and methods for administration of the compounds of Formula (I), Formula (II) or Formula (III), described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of such auxiliaries and methods of preparing sterile solutions of the present compositions are well known in the art, such as from (but not limited to) Gennaro, Ed., Remington's Pharmaceutical Sciences, 18th Edition, Mack Publishing Co. (Easton, Pa.) 1990 and the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

The compositions can also include a buffer or a pH-adjusting agent.

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intra-arterial, intravesical, oral, parenteral, rectal, sublingual, subcutaneous, transdermal or vaginal means.

It can be desirable to deliver the disclosed compounds to a subject over prolonged periods of time, for example, for periods of one week to one year from a single administration. Various slow release, depot or implant dosage forms can be utilized.

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of the compositions or pharmaceutical compositions disclosed herein from about 0.1 to 100 mg/kg or any range, value or fraction thereof per day, on at least one of day 1-40 of a treatment, or, alternatively or additionally, at least one of week 1-52 of treatment, using single, infusion or repeated doses. Alternatively or additionally, treatments can be provided for between 1-20 years, or any combination thereof.

A more detailed description of pharmaceutically acceptable excipients, formulations, dosages and methods of administration of the disclosed compositions and pharmaceutical compositions is disclosed in PCT Publication No. WO 2019/04981.

In an aspect, administration of the present compositions is systemic. Systemic administration can be any means known in the art and/or as described in detail herein.

Preferably, systemic administration is by an intravenous injection or an intravenous infusion. In one aspect, the administration is local. Local administration can be any means known in the art and/or as described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously.

### Methods of Use

The multivalent cationic terpene-derived cationic lipidoid compositions of the present disclosure can be used for gene delivery.

The disclosure provides a method for delivering a nucleic acid to a cell, tissue, organ, animal or subject. For example, provided are methods of using a compound of Formula (I), Formula (II) or Formula (III) complexed with DNA or RNA, or pharmaceutical compositions thereof, for delivery of RNA or DNA to a cell, tissue, organ, animal, or subject, as known in the art or as described herein, using the disclosed compositions and pharmaceutical compositions, e.g., administering or contacting the cell, tissue, organ, animal, or subject with an amount, e.g., a therapeutically effective amount, of the composition or pharmaceutical composition. In an aspect, the subject is a mammal. Preferably, the subject is human. The terms “subject” and “patient” are used interchangeably herein.

Any method can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one other agent.

Lipoplex nanoparticles comprising a compound of Formula (I), Formula (II) or Formula (III) and a nucleic acid, e.g., DNA or RNA, can be used for gene delivery following general methods disclosed for monopolar bolaamphiphilic lipids (e.g., see Martin et al., (2005) Current Pharmaceutical Design 11:375-394).

### EXAMPLES

### Protocol A Compounds

### Example 1—Preparation of Compound 1

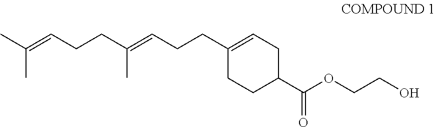 [SMILES: CC(C)=CCC/C(C)=C/CCC1=CCC(C(=O)OCCO)CC1]

### (E)-2-hydroxyethyl 4-(4,8-dimethylnona-3,7-dien-1-yl)cyclohex-3-enecarboxylate

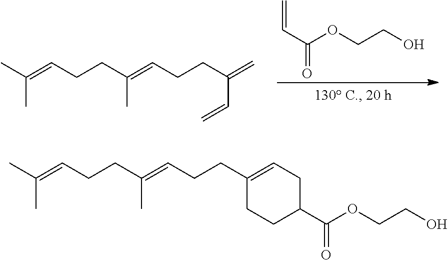

This compound was prepared as follows.

Trans-beta-farnesene (1.02 g, 4.99 mmol) and 2-hydroxyethyl acrylate (0.56 g, 4.99 mmol) were mixed in a glass tube and sealed. The reaction mixture was then stirred for 20 h at 130° C. The reaction mixture was cooled and purified by silica gel flash chromatography (eluent: 30% EtOAc/hexane).

Colorless oil; 1.31 g (86% yield). ¹H NMR (500 MHz, Chloroform-d) δ 5.42-5.31 (m, 1H), 5.12-5.00 (m, 211), 4.23-4.16 (m, 2H), 3.83-3.76 (m, 2H), 2.63-2.32 (m, 2H), 2.26-1.90 (m, 13H), 1.82-1.49 (m, 10H).

### Example 2—Preparation of Compound 3

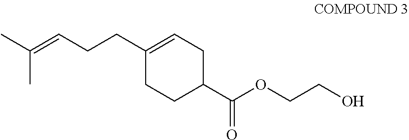 [SMILES: CC(C)=CCCC1=CCC(C(=O)OCCO)CC1]

### 2-hydroxyethyl 4-(4-methylpent-3-en-1-yl)cyclohex-3-enecarboxylate

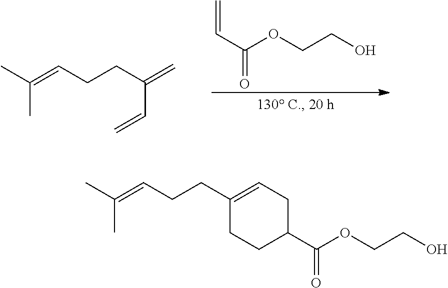

Myrcene (4.17 g, 30.6 mmol) and 2-hydroxyethyl acrylate (3.6 g, 30.61 mmol) were mixed in a glass tube and sealed. The reaction mixture was then stirred for 20 h at 130° C. The reaction mixture was cooled and purified by silica gel flash chromatography (eluent: 20% EtOAc/hexane).

Colorless oil; 5.4 g (77%). ¹H NMR (500 MHz, Chloroform-d) δ 5.40-5.28 (m, 1H), 5.09-4.99 (m, 1H), 4.24-4.11 (m, 2H), 3.84-3.70 (m, 2H), 2.67-2.46 (m, 2H), 2.33-1.83 (m, 9H), 1.72-1.48 (m, 7H).

### Protocol B Compounds

### Example 3—Preparation of Compound 2

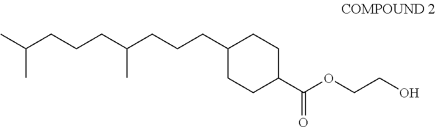 [SMILES: CC(C)CCCC(C)CCCC1CCC(C(=O)OCCO)CC1]

### 2-hydroxyethyl 4-(4,8-dimethylnonyl)cyclohexanecarboxylate

In a 100 mL round-bottom flask, compound 1 (2.1 g) was dissolved in a mixed solvent of ethanol and CH₂Cl₂ (4:1, 20 mL). To this solution, 10% Pd/C (210 mg) was added in one portion and the resulting dark suspension was stirred under a hydrogen atmosphere for 24 h at room temperature. The reaction mixture was filtered through celite and rinsed with CH₂Cl₂ (3×50 mL). The filtrate was evaporated to give compound 2 as a colorless oil.

Colorless oil; 2.06 g (97%), 5.4 g (77%). ¹H NMR (500 MHz, Chloroform-d) δ 4.20-4.06 (m, 2H), 3.80-3.68 (m, 2H), 3.18-2.71 (m, 1H), 2.66-2.13 (m, 1H), 2.01-1.59 (m, 3H), 1.58-0.93 (m, 18H), 0.91-0.57 (m, 11H).

### Example 4—Preparation of Compound 4

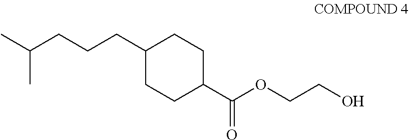 [SMILES: CC(C)CCCC1CCC(C(=O)OCCO)CC1]

### 2-hydroxyethyl 4-(4-methylpentyl)cyclohexanecarboxylate

In a 100 mL round-bottom flask, compound 3 (0.51 g) was dissolved in a mixed solvent of ethanol and CH₂Cl₂ (4:1, 20 mL). To this solution, 10% Pd/C (51 mg) was added in one portion and the resulting dark suspension was stirred under a hydrogen atmosphere for 24 h at room temperature. The reaction mixture was filtered through celite and rinsed with CH₂Cl₂ (3×50 mL). The filtrate was evaporated to give compound 4 as a colorless oil.

Colorless oil; 0.46 g (97%). ¹H NMR (500 MHz, Chloroform-d) δ 4.26-4.11 (m, 2H), 3.78 (s, 2H), 2.71-2.12 (m, 2H), 2.01-1.86 (m, 2H), 1.85-1.65 (m, 1H), 1.62-0.96 (m, 12H), 0.94-0.74 (m, 7H).

### Protocol C Compounds

### Example 5—Preparation of Compound FA

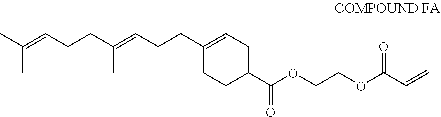 [SMILES: C=CC(=O)OCCOC(=O)C1CC=C(CC/C=C(\C)CCC=C(C)C)CC1]

### (E)-2-(acryloyloxy)ethyl 4-(4,8-dimethylnona-3,7-dien-1-yl)cyclohex-3-enecarboxylate

Compound 1 (112 mg, 0.35 mmol) was mixed with triethylamine (0.1 mL, 0.7 mmol) in anhydrous CH₂Cl₂ (5.0 mL) and the solution was cooled to 0 C with an ice/water bath. Acryloyl chloride (0.06 mL, 0.7 mmol) was added to the cooled solution dropwise over a period of 10 minutes. The resulting solution was stirred for 20 h allowing the temperature to rise to room temperature. Added saturated solution of sodium bicarbonate (20 mL) and CH₂Cl₂ (20 mL). Both layers were separated, the aqueous layer was extracted with CH₂Cl₂ (3×20 mL).

Combined organic extracts were washed with brine (20 mL); dried over sodium sulfate; filtered; and evaporated on rota vapor. The crude was purified by silica gel flash chromatography (eluent: 15% EtOAc/hexane).

Colorless oil; 126 mg (97%). ¹H NMR (500 MHz, Chloroform-d) δ 6.43 (dd, J=17.3, 1.6 Hz, 1H), 6.14 (ddd, J=17.3, 10.4, 1.7 Hz, 1H), 5.86 (dd, J=10.4, 1.5 Hz, 1H), 5.43-5.34 (m, 1H), 5.14-5.04 (m, 2H), 4.43-4.27 (m, 4H), 2.65-2.46 (m, 1H), 2.31-1.91 (m, 13H), 1.74-1.56 (m, 10H).

### Example 6—Preparation of Compound MA

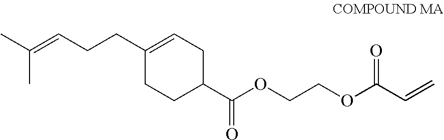 [SMILES: C=CC(=O)OCCOC(=O)C1CC=C(CCC=C(C)C)CC1]

### 2-(acryloyloxy)ethyl 4-(4-methylpent-3-en-1-yl)cyclohex-3-enecarboxylate

Compound 3 (0.46 g, 1.82 mmol) was mixed with triethylamine (0.76 mL, 3.6 mmol) in anhydrous CH₂Cl₂ (10.0 mL) and the solution was cooled to 0 C with an ice/water bath. Acryloyl chloride (0.45 mL, 3.6 mmol) was added to the cooled solution dropwise over a period of 10 minutes. The resulting solution was stirred for 20 h allowing the temperature to rise to room temperature. Added saturated solution of sodium bicarbonate (20 mL) and CH₂Cl₂ (20 mL). Both layers were separated, the aqueous layer was extracted with CH₂Cl₂ (3×20 mL). Combined organic extracts were washed with brine (20 mL); dried over sodium sulfate; filtered; and evaporated on rota vapor. The crude was purified by silica gel flash chromatography (eluent: 10% EtOAc/hexane).

Colorless oil; 417 mg (74%). ¹H NMR (500 MHz, Chloroform-d) δ 6.48-6.37 (m, 1H), 6.19-6.07 (m, 1H), 5.86 (dd, J=10.4, 1.4 Hz, 1H), 5.44-5.33 (m, 1H), 5.15-5.03 (m, 1H), 4.42-4.27 (m, 4H), 2.65-2.48 (m, 1H), 2.33-1.90 (m, 9H), 1.78-1.54 (m, 7H).

### Example 7—Preparation of Compound HFA

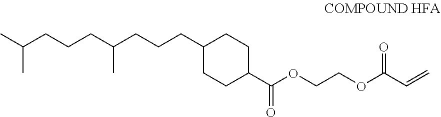 [SMILES: C=CC(=O)OCCOC(=O)C1CCC(CCCC(C)CCCC(C)C)CC1]

### 2-(acryloyloxy)ethyl 4-(4,8-dimethylnonyl)cyclohexanecarboxylate

Compound HFA was prepared in accordance with the General Procedure C. The crude was purified by silica gel flash chromatography (eluent: 15% EtOAc/hexane).

Colorless oil; ¹H NMR (500 MHz, Chloroform-d) δ 6.43 (dt, J=17.3, 1.3 Hz, 1H), 6.20-6.06 (m, 1H), 5.86 (ddd, J=10.6, 2.4, 1.5 Hz, 1H), 4.40-4.25 (m, 4H), 2.73-2.12 (m, 1H), 2.05-1.68 (m, 3H), 1.62-1.47 (m, 3H), 1.47-1.01 (m, 16H), 0.98-0.81 (m, 10H).

### Example 8—Preparation of Compound HMA

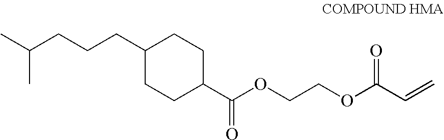 [SMILES: C=CC(=O)OCCOC(=O)C1CCC(CCCC(C)C)CC1]

### 2-(acryloyloxy)ethyl 4-(4-methylpentyl)cyclohexanecarboxylate

Compound HMA was prepared in accordance with the General Procedure C. The crude was purified by silica gel flash chromatography (eluent: 10% EtOAc/hexane).

Colorless oil; ¹H NMR (500 MHz, Chloroform-d) δ 6.42 (dd, J=17.3, 1.4 Hz, 1H), 6.13 (dd, J=17.3, 10.5 Hz, 1H), 5.85 (dt, J=10.4, 1.8 Hz, 1H), 4.41-4.26 (m, 4H), 2.71-2.15 (m, 1H), 2.03-1.86 (m, 2H), 1.85-1.60 (m, 2H), 1.59-0.96 (m, 11H), 0.95-0.69 (m, 7H).

### Protocol D Compounds

### Example 9—Preparation of Compound MA-404

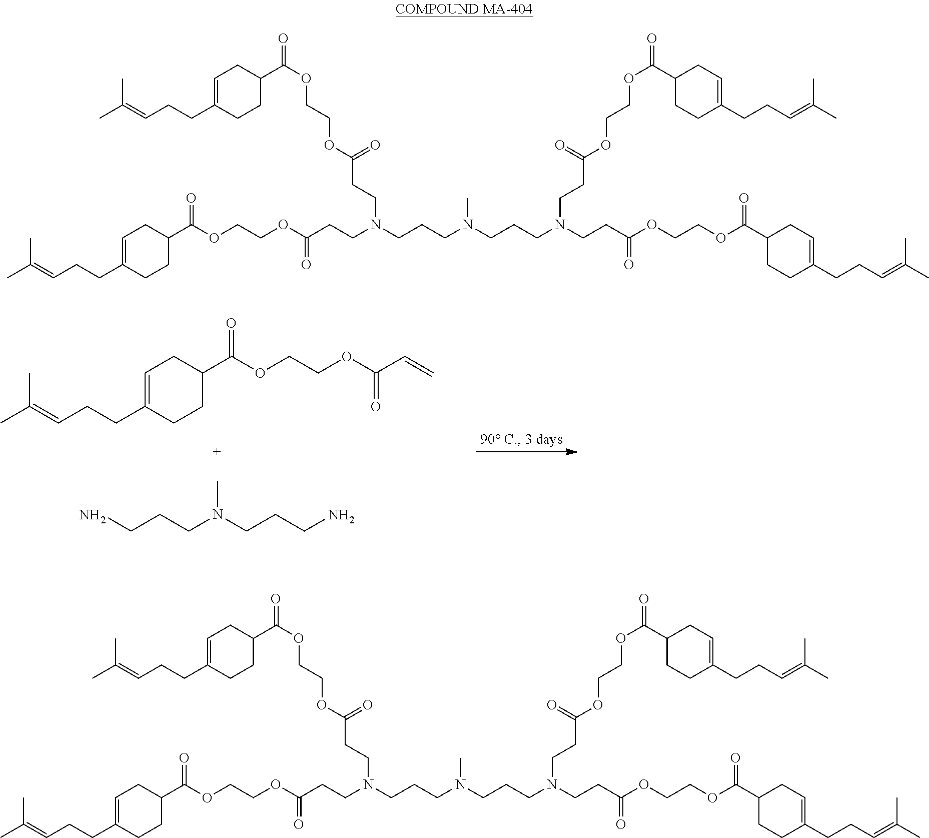

Compound MA (337 mg, 1.0 mmol) was mixed with amine 404 (28.8 mg, 0.2 mmol) in a 4-dram glass vial. The capped reaction vial was stirred at 90° C. for 3 days. The cooled reaction mixture was purified by silica gel flash chromatography (eluent: 5% MeOH/CH₂Cl₂).

Pale-yellow oil; 270 mg (99%). ¹H NMR (400 MHz, Chloroform-d) δ 5.41-5.32 (m, 4H), 5.13-5.01 (m, 4H), 4.36-4.21 (m, 16H), 2.93-2.78 (m, 3H), 2.72 (t, J=6.8 Hz, 7H), 2.66-2.32 (m, 17H), 2.30-1.75 (m, 43H), 1.70-1.50 (m, 29H).

### Example 10—Preparation of Compound MA-201

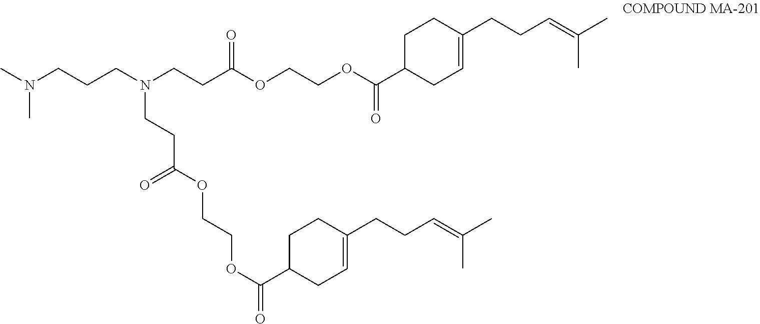 [SMILES: CC(C)=CCCC1=CCC(C(=O)OCCOC(=O)CCN(CCCN(C)C)CCC(=O)OCCOC(=O)C2CC=C(CCC=C(C)C)CC2)CC1]

### ((3,3′-((3-(dimethylamino)propyl)azanediyl)bis(propanoyl))bis(oxy))bis(ethane-2,1-diyl) bis(4-(4-methylpent-3-en-1-yl)cyclohex-3-enecarboxylate)

Compound MA-201 was prepared in accordance with the General Procedure D. The crude was purified by silica gel flash chromatography (eluent: 50% EtOAc/hexane). ¹H NMR (400 MHz, Chloroform-d) δ 5.45-5.31 (m, 2H), 5.14-5.00 (m, 2H), 4.40-4.15 (m, 8H), 3.01-2.39 (m, 14H), 2.36-1.84 (m, 24H), 1.78-1.46 (m, 16H).

### Example 11—Preparation of Compound MA-202

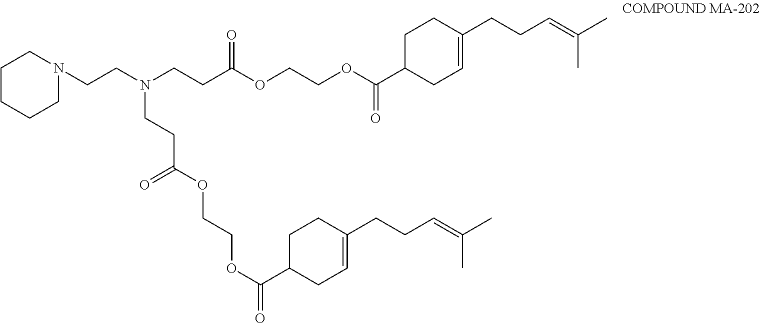 [SMILES: CC(C)=CCCC1=CCC(C(=O)OCCOC(=O)CCN(CCC(=O)OCCOC(=O)C2CC=C(CCC=C(C)C)CC2)CCN2CCCCC2)CC1]

### ((3,3′-((2-(piperidin-1-yl)ethyl)azanediyl)bis(propanoyl))bis(oxy))bis(ethane-2,1-diyl) bis(4-(4-methylpent-3-en-1-yl)cyclohex-3-enecarboxylate)

Compound MA-202 may be prepared in accordance with the General Procedure D.

### Example 12—Preparation of Compound MA-401

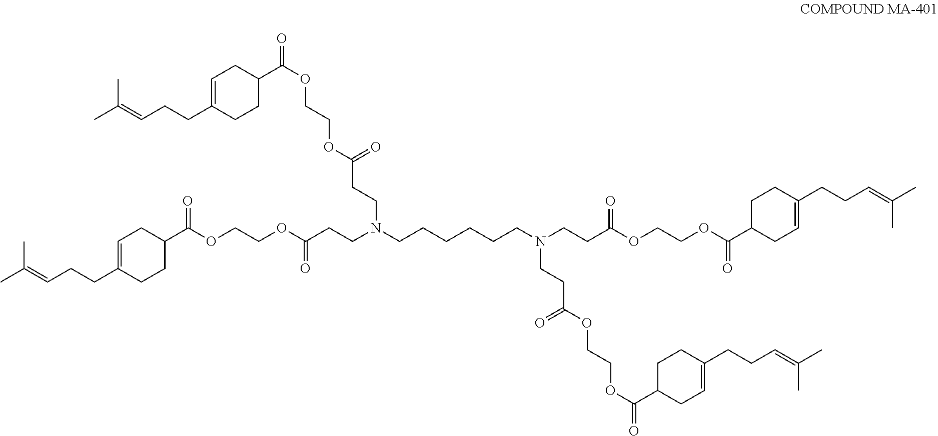 [SMILES: CC(C)=CCCC1=CCC(C(=O)OCCOC(=O)CCN(CCCCCCN(CCC(=O)OCCOC(=O)C2CC=C(CCC=C(C)C)CC2)CCC(=O)OCCOC(=O)C2CC=C(CCC=C(C)C)CC2)CCC(=O)OCCOC(=O)C2CC=C(CCC=C(C)C)CC2)CC1]

Compound MA-401 was prepared in accordance with the General Procedure D. The crude was purified by silica gel flash chromatography (eluent: 5% MeOH/DCM). ¹H NMR (400 MHz, Chloroform-d) δ 5.41-5.32 (m, 4H), 5.10-5.02 (m, 4H), 4.40-4.18 (m, 16H), 3.04-2.67 (m, 6H), 2.64-2.34 (m, 14H), 2.27-1.87 (m, 40H), 1.67-1.20 (m, 36H).

### Example 13—Preparation of Compound MA-402

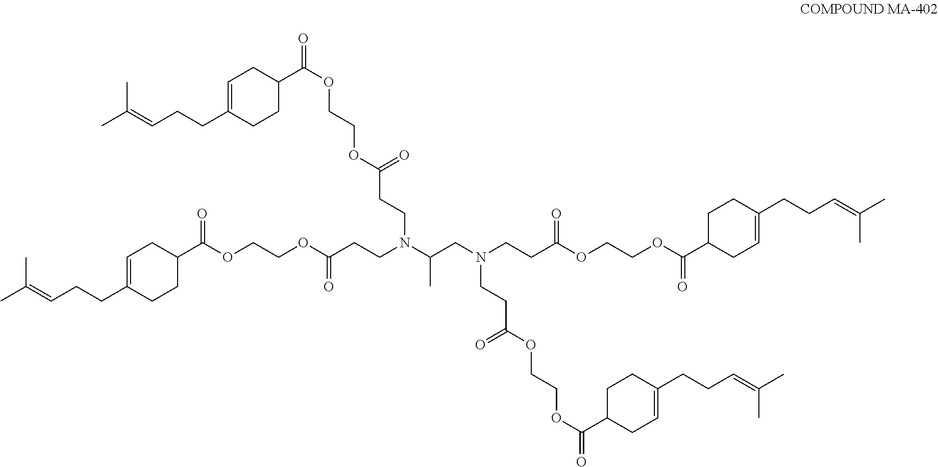 [SMILES: CC(C)=CCCC1=CCC(C(=O)OCCOC(=O)CCN(CCC(=O)OCCOC(=O)C2CC=C(CCC=C(C)C)CC2)CC(C)N(CCC(=O)OCCOC(=O)C2CC=C(CCC=C(C)C)CC2)CCC(=O)OCCOC(=O)C2CC=C(CCC=C(C)C)CC2)CC1]

Compound MA-402 was prepared in accordance with the General Procedure D. The crude was purified by silica gel flash chromatography (eluent: 5% MeOH/DCM). ¹H NMR (400 MHz, Chloroform-d) δ 5.47-5.28 (m, 4H), 5.14-4.99 (m, 4H), 4.33-4.20 (m, 16H), 2.87-2.35 (m, 20H), 2.26-1.88 (m, 42H), 1.69-1.54 (m, 28H).

### Example 14—Preparation of Compound MA-403

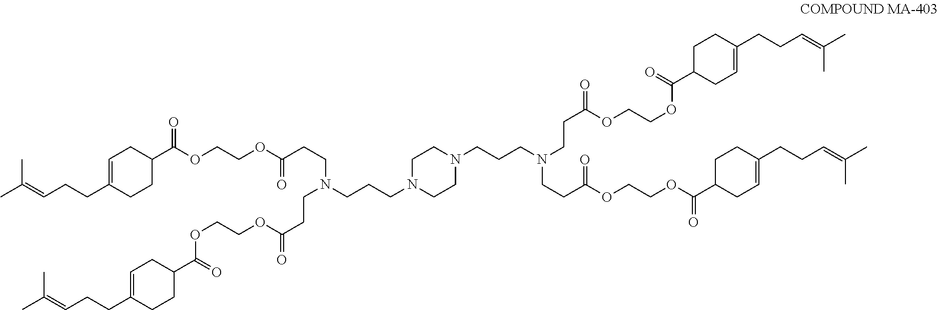 [SMILES: CC(C)=CCCC1=CCC(C(=O)OCCOC(=O)CCN(CCCN2CCN(CCCN(CCC(=O)OCCOC(=O)C3CC=C(CCC=C(C)C)CC3)CCC(=O)OCCOC(=O)C3CC=C(CCC=C(C)C)CC3)CC2)CCC(=O)OCCOC(=O)C2CC=C(CCC=C(C)C)CC2)CC1]

Compound MA-403 was prepared in accordance with the General Procedure D. The crude was purified by silica gel flash chromatography (eluent: 5% MeOH/DCM). ¹H NMR (400 MHz, Chloroform-d) δ 5.41-5.28 (m, 4H), 5.11-4.95 (m, 4H), 4.36-4.17 (m, 16H), 2.71 (t, J=7.0 Hz, 8H), 2.61-2.26 (m, 24H), 2.23-1.76 (m, 40H), 1.69-1.46 (m, 32H).

### Example 15—Preparation of Compound MA-601

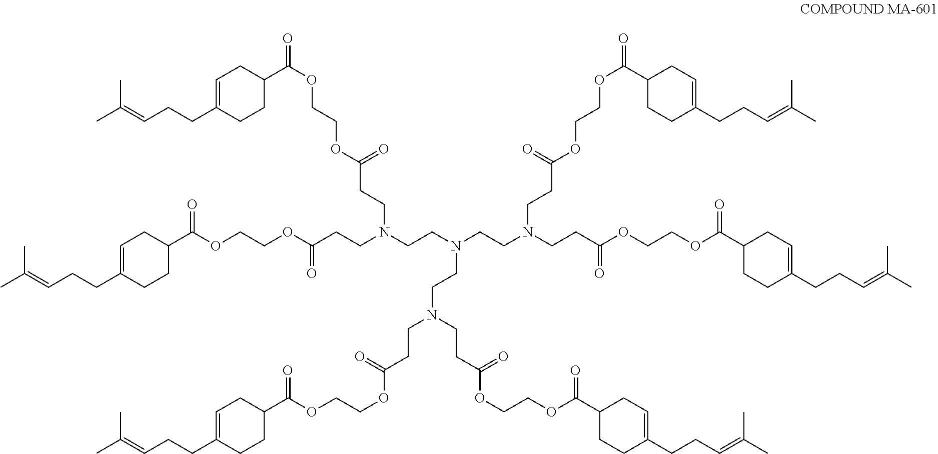 [SMILES: CC(C)=CCCC1=CCC(C(=O)OCCOC(=O)CCN(CCC(=O)OCCOC(=O)C2CC=C(CCC=C(C)C)CC2)CCN(CCN(CCC(=O)OCCOC(=O)C2CC=C(CCC=C(C)C)CC2)CCC(=O)OCCOC(=O)C2CC=C(CCC=C(C)C)CC2)CCN(CCC(=O)OCCOC(=O)C2CC=C(CCC=C(C)C)CC2)CCC(=O)OCCOC(=O)C2CC=C(CCC=C(C)C)CC2)CC1]

Compound MA-601 was prepared in accordance with the General Procedure D. The crude was purified by silica gel flash chromatography (eluent: 10% MeOH/DCM). ¹H NMR (400 MHz, Chloroform-d) δ 5.43-5.29 (m, 6H), 5.14-4.97 (m, 6H), 4.37-4.12 (m, 24H), 2.75 (t, J=9.6, 4.5 Hz, 12H), 2.67-2.32 (m, 24H), 2.29-1.76 (m, 58H), 1.69-1.46 (m, 44H).

### Example 16—Preparation of Compound HMA-201

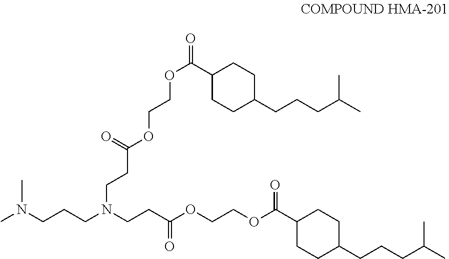 [SMILES: CC(C)CCCC1CCC(C(=O)OCCOC(=O)CCN(CCCN(C)C)CCC(=O)OCCOC(=O)C2CCC(CCCC(C)C)CC2)CC1]

Compound HMA-201 was prepared in accordance with the General Procedure D. The crude was purified by silica gel flash chromatography (eluent: 50% EtOAc/hexane). ¹H NMR (400 MHz, Chloroform-d) δ 4.37-4.19 (m, 8H), 2.83-2.75 (m, 3H), 2.75-2.62 (m, 3H), 2.50-2.40 (m, 2H), 2.37-2.15 (m, 2H), 2.04-1.67 (m, 10H), 1.60-1.33 (m, 8H), 1.29-1.07 (m, 20H), 0.87-0.77 (m, 18H).

### Example 17—Preparation of Compound HMA-202

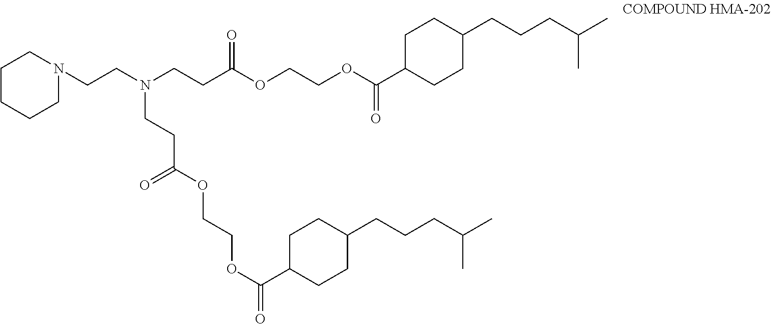 [SMILES: CC(C)CCCC1CCC(C(=O)OCCOC(=O)CCN(CCC(=O)OCCOC(=O)C2CCC(CCCC(C)C)CC2)CCN2CCCCC2)CC1]

Compound HMA-202 was prepared in accordance with the General Procedure D. The crude was purified by silica gel flash chromatography (eluent: 30% EtOAc/hexane). ¹H NMR (400 MHz, Chloroform-d) δ 4.33-4.16 (m, 8H), 3.21-2.89 (m, 6H), 2.75 (t, J=6.5 Hz, 4H), 2.54-2.43 (m, 4H), 2.37-2.14 (m, 2H), 2.09-1.73 (m, 10H), 1.69-0.96 (m, 28H), 0.83 (dd, J=6.6, 1.1 Hz, 16H).

### Example 18—Preparation of Compound HMA-401

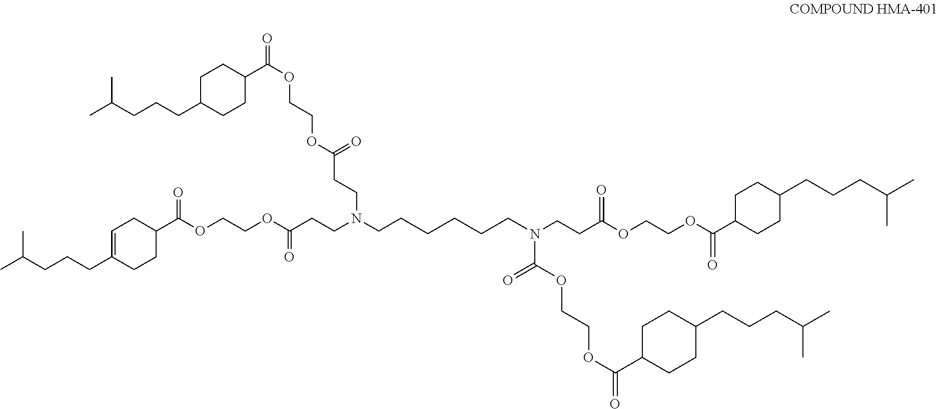 [SMILES: CC(C)CCCC1=CCC(C(=O)OCCOC(=O)CCN(CCCCCCN(CCC(=O)OCCOC(=O)C2CCC(CCCC(C)C)CC2)C(=O)OCCOC(=O)C2CCC(CCCC(C)C)CC2)CCC(=O)OCCOC(=O)C2CCC(CCCC(C)C)CC2)CC1]

Compound HMA-401 was prepared in accordance with the General Procedure D. The crude was purified by silica gel flash chromatography (eluent: 5% MeOH/DCM). ¹H NMR (400 MHz, Chloroform-d) δ 4.36-4.14 (m, 16H), 2.75 (t, J=7.3 Hz, 6H), 2.66-2.14 (m, 16H), 1.98-1.68 (m, 14H), 1.58-1.05 (m, 54H), 0.83 (dd, J=6.7, 1.3 Hz, 30H).

### Example 19—Preparation of Compound HMA-402

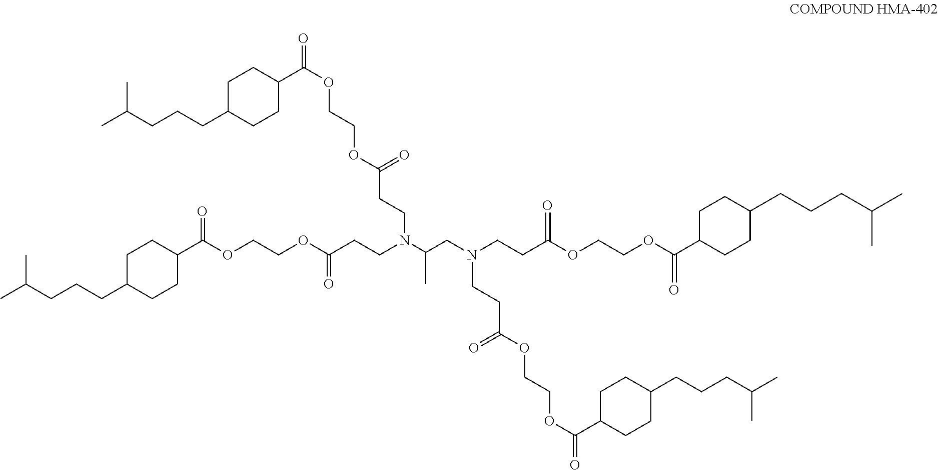 [SMILES: CC(C)CCCC1CCC(C(=O)OCCOC(=O)CCN(CCC(=O)OCCOC(=O)C2CCC(CCCC(C)C)CC2)CC(C)N(CCC(=O)OCCOC(=O)C2CCC(CCCC(C)C)CC2)CCC(=O)OCCOC(=O)C2CCC(CCCC(C)C)CC2)CC1]

Compound HMA-402 was prepared in accordance with the General Procedure D. The crude was purified by silica gel flash chromatography (eluent: 5% MeOH/DCM). ¹H NMR (400 MHz, Chloroform-d) δ 4.40-4.15 (m, 16H), 2.97-2.40 (m, 12H), 2.39-2.15 (m, 5H), 2.03-1.67 (m, 15H), 1.63-0.98 (m, 51H), 0.91-0.74 (m, 31H).

### Example 20—Preparation of Compound HMA-403

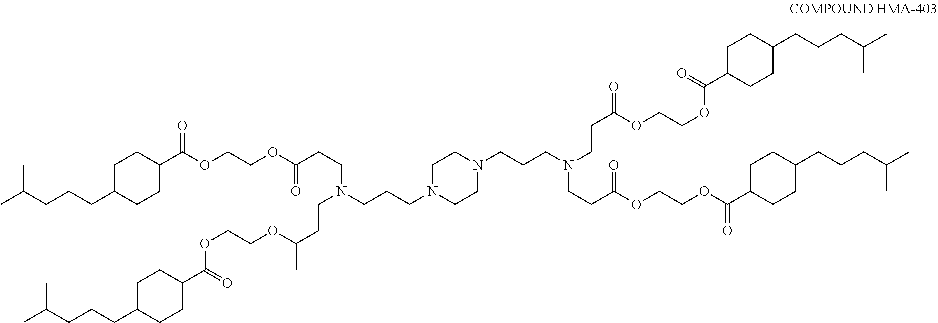 [SMILES: CC(C)CCCC1CCC(C(=O)OCCOC(=O)CCN(CCCN2CCN(CCCN(CCC(=O)OCCOC(=O)C3CCC(CCCC(C)C)CC3)CCC(C)OCCOC(=O)C3CCC(CCCC(C)C)CC3)CC2)CCC(=O)OCCOC(=O)C2CCC(CCCC(C)C)CC2)CC1]

Compound HMA-403 was prepared in accordance with the General Procedure D. The crude was purified by silica gel flash chromatography (eluent: 5% MeOH/DCM). ¹H NMR (400 M Hz, Chloroform-d) δ 4.35-4.15 (m, 16H), 2.71 (t, J=7.1 Hz, 8H), 2.62-2.05 (m, 26H), 1.97-0.95 (m, 62H), 0.93-0.61 (m, 3211).

### Example 21—Preparation of Compound HMA-404

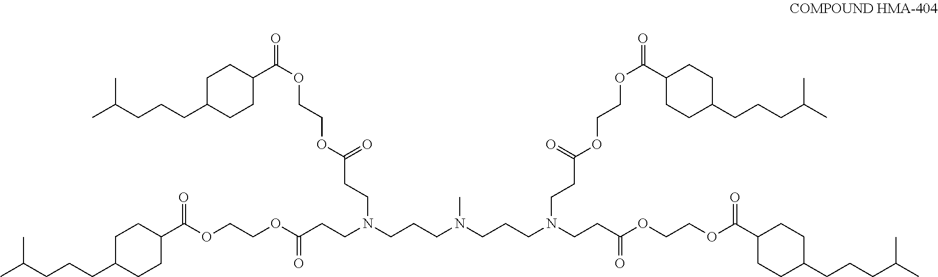 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

Compound HMA-404 was prepared in accordance with the General Procedure D. The crude was purified by silica gel flash chromatography (eluent: 5% MeOH/DCM). ¹H NMR (400 MHz, Chloroform-d) δ 4.38-4.12 (m, 16H), 2.90-2.78 (m, 4H), 2.71 (t, J=6.8 Hz, 8H), 2.61 (s, 3H), 2.57-2.36 (m, 12H), 2.35-2.04 (m, 4H), 2.01-1.62 (m, 17H), 1.60-0.96 (m, 45H), 0.94-0.67 (m, 30H).

### Example 22—Preparation of Compound HMA-601

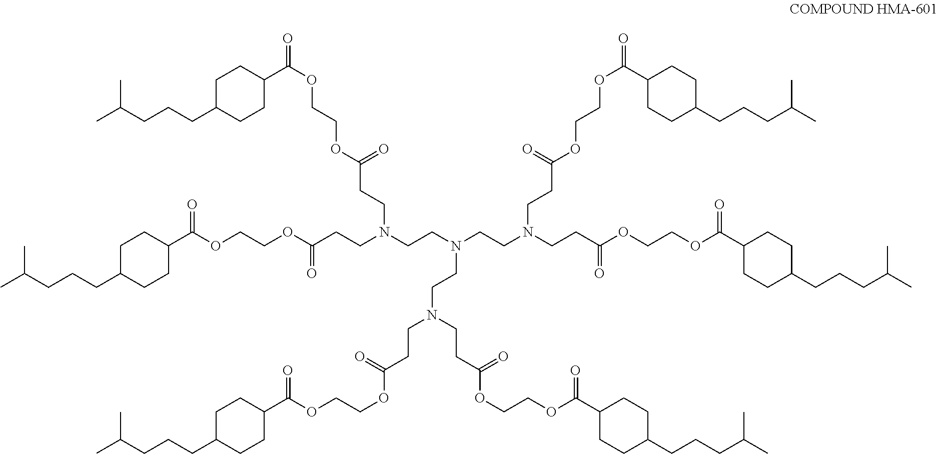 [SMILES: CC(C)CCCC1CCC(C(=O)OCCOC(=O)CCN(CCC(=O)OCCOC(=O)C2CCC(CCCC(C)C)CC2)CCN(CCN(CCC(=O)OCCOC(=O)C2CCC(CCCC(C)C)CC2)CCC(=O)OCCOC(=O)C2CCC(CCCC(C)C)CC2)CCN(CCC(=O)OCCOC(=O)C2CCC(CCCC(C)C)CC2)CCC(=O)OCCOC(=O)C2CCC(CCCC(C)C)CC2)CC1]

Compound HMA-601 was prepared in accordance with the General Procedure D. The crude was purified by silica gel flash chromatography (eluent: 10% MeOH/DCM). ¹H NMR (400 MHz, Chloroform-d) δ 4.36-4.13 (m, 24H), 2.76 (t, J=7.1 Hz, 12H), 2.66-2.37 (m, 24H), 2.35-2.13 (m, 6H), 1.98-1.65 (m, 22H), 1.58-1.00 (m, 68H), 0.83 (dd, J=6.6, 1.1 Hz, 42H).

### Example 23—Preparation of Compound FA-201

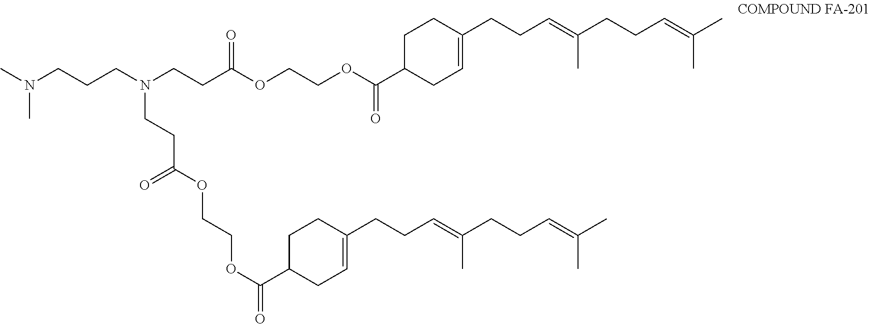 [SMILES: CC(C)=CCC/C(C)=C/CCC1=CCC(C(=O)OCCOC(=O)CCN(CCCN(C)C)CCC(=O)OCCOC(=O)C2CC=C(CC/C=C(\C)CCC=C(C)C)CC2)CC1]

### (E)-((3,3′-((3-(dimethylamino)propyl)azanediy)bis(propanoyl))bis(oxy))bis(ethane-2,1-diyl) bis(4-((E)-4,8-dimethylnona-3,7-dien-1-yl)cyclohex-3-enecarboxylate)

Compound FA-201 was prepared in accordance with the General Procedure D. The crude was purified by silica gel flash chromatography (eluent: 50% EtOAc/hexane). ¹H NMR (400 MHz, Chloroform-d) δ 5.43-5.30 (m, 2H), 5.17-4.95 (m, 4H), 4.38-4.15 (m, 8H), 3.93-3.75 (m, 1H), 3.64-3.03 (m, 2H), 2.97-2.79 (m, 2H), 2.76-2.62 (m, 8H), 2.59-2.31 (m, 6H), 2.29-1.76 (m, 28H), 1.75-1.35 (m, 21H).

### Example 24—Preparation of Compound FA-202

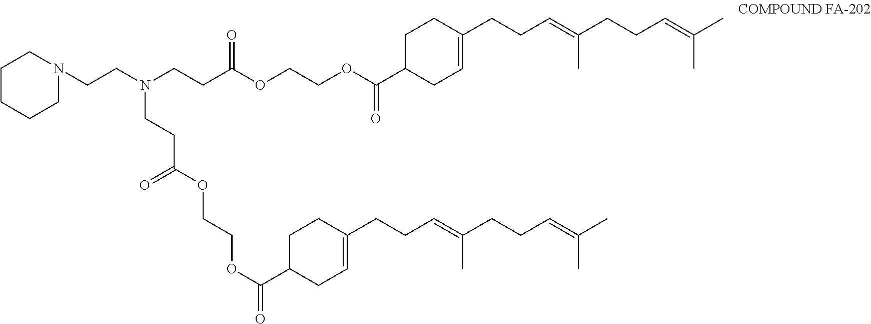 [SMILES: CC(C)=CCC/C(C)=C/CCC1=CCC(C(=O)OCCOC(=O)CCN(CCC(=O)OCCOC(=O)C2CC=C(CC/C=C(\C)CCC=C(C)C)CC2)CCN2CCCCC2)CC1]

### (E)-((3,3′-((2-(piperidin-1-yl)ethyl)azanediyl)bis(propanoyl))bis(oxy))bis(ethane-2,1-diyl) bis(4-((E)-4,8-dimethylnona-3,7-dien-1-yl)cyclohex-3-enecarboxylate)

Compound FA-202 was prepared in accordance with the General Procedure D. The crude was purified by silica gel flash chromatography (eluent: 30% EtOAc/hexane). ¹H NMR (400 MHz, Chloroform-d) δ 5.45-5.31 (m, 2H), 5.16-5.01 (m, 4H), 4.26 (dt, J=5.1, 3.0 Hz, 8H), 3.04 (dt, J=10.5, 5.3 Hz, 6H), 2.77 (t, J=6.5 Hz, 4H), 2.50 (t, J=6.4 Hz, 6H), 2.31-1.75 (m, 32H), 1.74-1.28 (m, 24H).

### Example 25—Preparation of Compound FA-401

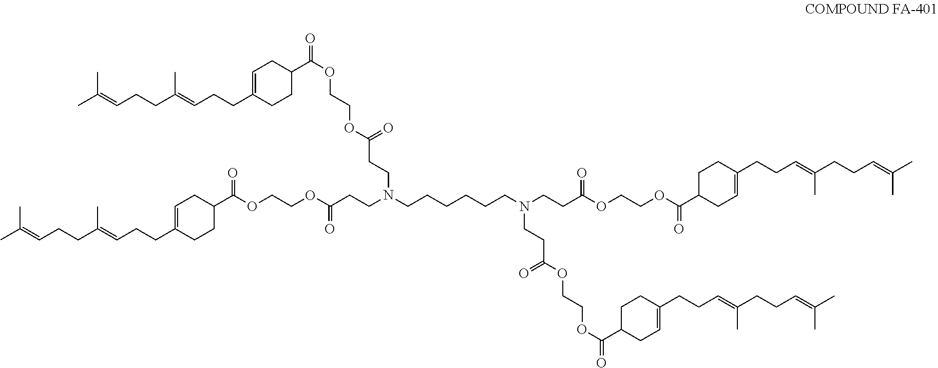 [SMILES: CC(C)=CCC/C(C)=C/CCC1=CCC(C(=O)OCCOC(=O)CCN(CCCCCCN(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CC1]

Compound FA-401 was prepared in accordance with the General Procedure D. The crude was purified by silica gel flash chromatography (eluent: 5% MeOH/DCM). ¹H NMR (400 MHz, Chloroform-d) δ 5.44-5.31 (m, 4H), 5.15-5.02 (m, 8H), 4.27 (s, 16H), 2.76 (t, J=7.3 Hz, 6H), 2.66-2.35 (m, 15H), 2.31-1.86 (m, 55H), 1.69-1.18 (m, 48H).

### Example 26—Preparation of Compound FA-402

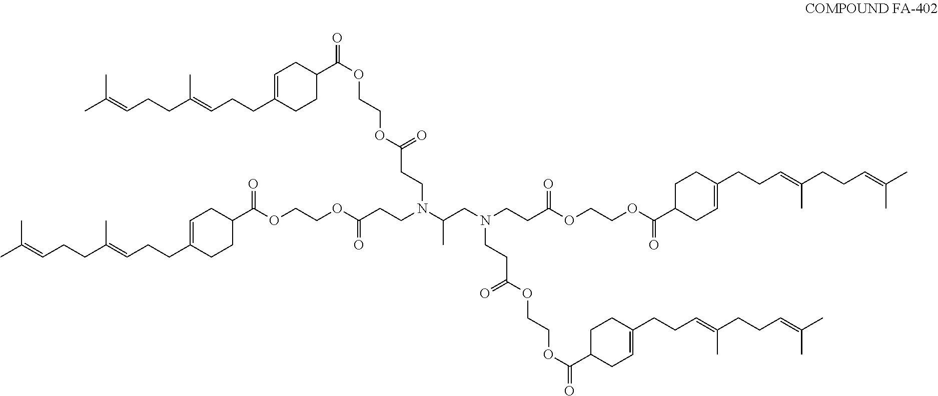 [SMILES: CC(C)=CCC/C(C)=C/CCC1=CCC(C(=O)OCCOC(=O)CCN(CCC(=O)OCCOC(=O)C2CC=C(CC/C=C(\C)CCC=C(C)C)CC2)CC(C)N(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CC1]

Compound FA-402 was prepared in accordance with the General Procedure D. The crude was purified by silica gel flash chromatography (eluent: 5% MeOH/DCM). H NMR (400 MHz, Chloroform-d) δ 5.45-5.31 (m, 4H), 5.16-5.01 (m, 8H), 4.43-4.15 (m, 16H), 3.03-2.31 (m, 21H), 2.30-1.78 (m, 56H), 1.77-1.46 (m, 41H).

### Example 27—Preparation of Compound FA-403

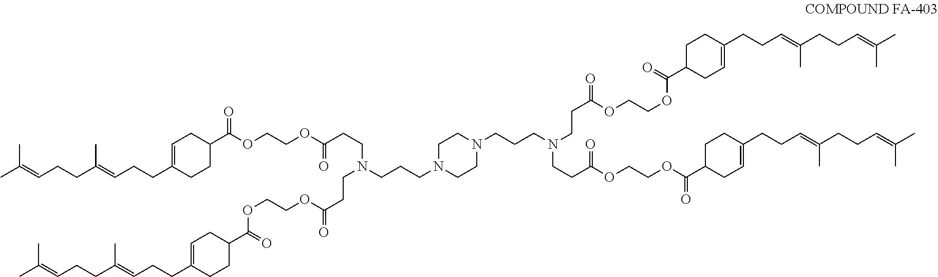 [SMILES: CC(C)=CCC/C(C)=C/CCC1=CCC(C(=O)OCCOC(=O)CCN(CCCN2CCN(CCCN(CCC(=O)OCCOC(=O)C3CC=C(CC/C=C(\C)CCC=C(C)C)CC3)CCC(=O)OCCOC(=O)C3CC=C(CC/C=C(\C)CCC=C(C)C)CC3)CC2)CCC(=O)OCCOC(=O)C2CC=C(CC/C=C(\C)CCC=C(C)C)CC2)CC1]

### ((3,3′,3″,3′″-((piperazine-1,4-diylbis(propane-3,1-diyl))bis(azanetriyl))tetrakis(propanoyl))tetrakis(oxy))tetrakis(ethane-2,1-diyl) tetrakis(4-((E)-4,8-dimethylnona-3,7-dien-1-yl)cyclohex-3-enecarboxylate)

Compound FA-403 was prepared in accordance with the General Procedure D. The crude was purified by silica gel flash chromatography (eluent: 5% MeOH/DCM). ¹H NMR (400 MHz, Chloroform-d) δ 5.44-5.30 (m, 4H), 5.15-4.98 (m, 8H), 4.26 (s, 16H), 2.73 (t, J=7.0 Hz, 8H), 2.68-2.29 (m, 25H), 2.28-1.81 (m, 55H), 1.80-1.44 (m, 44H).

### Example 28—Preparation of Compound FA-404

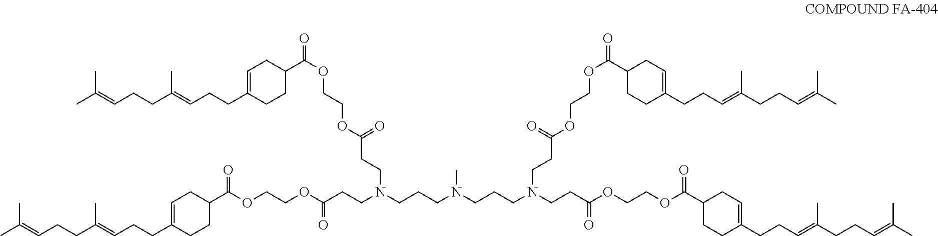 [SMILES: CC(C)=CCC/C(C)=C/CCC1=CCC(C(=O)OCCOC(=O)CCN(CCCN(C)CCCN(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CC1]

Compound FA-404 was prepared in accordance with the General Procedure D. The crude was purified by silica gel flash chromatography (eluent: 5% MeOH/DCM). ¹H NMR (400 MHz, Chloroform-d) δ 5.47-5.29 (m, 4H), 5.15-5.01 (m, 8H), 4.39-4.15 (m, 16H), 2.97-2.64 (m, 8H), 2.63-2.34 (m, 14H), 2.32-1.74 (m, 61H), 1.72-1.31 (m, 44H).

### Example 29—Preparation of Compound FA-601

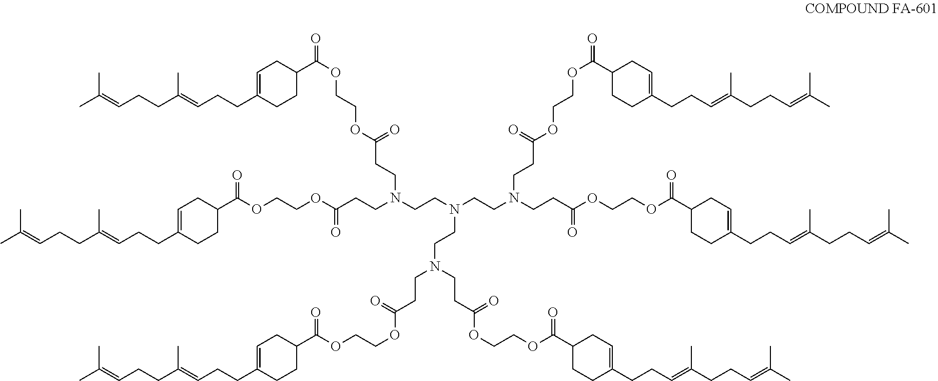 [SMILES: CC(C)=CCC/C(C)=C/CCC1=CCC(C(=O)OCCOC(=O)CCN(CCC(=O)OCCOC(=O)C2CC=C(CC/C=C(\C)CCC=C(C)C)CC2)CCN(CCN(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CCN(CCC(=O)OCCOC(=O)C2CC=C(CC/C=C(\C)CCC=C(C)C)CC2)CCC(=O)OCCOC(=O)C2CC=C(CC/C=C(\C)CCC=C(C)C)CC2)CC1]

Compound FA-601 was prepared in accordance with the General Procedure D. The crude was purified by silica gel flash chromatography (eluent: 10% MeOH/DCM). ¹H NMR (400 MHz, Chloroform-d) δ 5.45-5.30 (m, 6H), 5.15-4.97 (m, 12H), 4.26 (s, 24H), 2.77 (t, J=7.1 Hz, 12H), 2.69-2.30 (m, 27H), 2.28-1.77 (m, 80H), 1.74-1.28 (m, 61H).

### Example 30—Preparation of Compound HFA-201

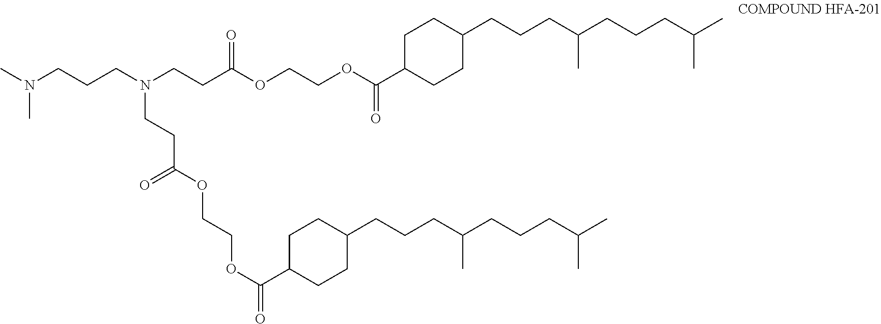 [SMILES: CC(C)CCCC(C)CCCC1CCC(C(=O)OCCOC(=O)CCN(CCCN(C)C)CCC(=O)OCCOC(=O)C2CCC(CCCC(C)CCCC(C)C)CC2)CC1]

### ((3,3′-((3-(dimethylamino)propyl)azanediyl)bis(propanoyl))bis(oxy))bis(ethane-2,1-diyl) bis(4-(4,8-dimethylnonyl)cyclohexanecarboxylate)

Compound HFA-201 was prepared in accordance with the General Procedure D. The crude was purified by silica gel flash chromatography (eluent: 50% EtOAc/hexane). ¹H NMR (400 MHz, Chloroform-d) δ 4.46-4.12 (m, 8H), 2.98-2.32 (m, 12H), 2.01-1.68 (m, 8H), 1.60-0.96 (m, 43H), 0.92-0.73 (m, 23H).

### Example 31—Preparation of Compound HFA-202

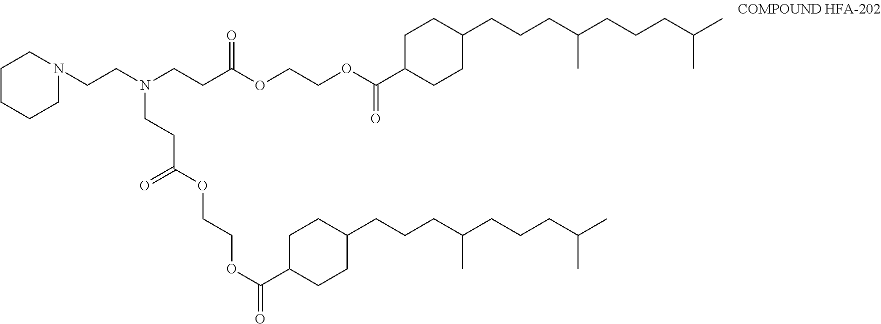 [SMILES: CC(C)CCCC(C)CCCC1CCC(C(=O)OCCOC(=O)CCN(CCC(=O)OCCOC(=O)C2CCC(CCCC(C)CCCC(C)C)CC2)CCN2CCCCC2)CC1]

### ((3,3′-((2-(piperidin-1-yl)ethyl)azanediyl)bis(propanoyl))bis(oxy))bis(ethane-2,1-diyl) bis(4-(4,8-dimethylnonyl)cyclohexanecarboxylate)

Compound HFA-202 was prepared in accordance with the General Procedure D. The crude was purified by silica gel flash chromatography (eluent: 30% EtOAc/hexane). ¹H NMR (400 MHz, Chloroform-d) δ 4.31-4.20 (m, 8H), 3.04 (dd, J=11.6, 6.1, 5.1 Hz, 8H), 2.76 (t, J=6.5 Hz, 4H), 2.49 (t, J=6.6, 2.0 Hz, 4H), 2.11-1.68 (m, 12H), 1.67-0.95 (m, 40H), 0.92-0.64 (m, 22H).

### Example 32—Preparation of Compound HFA-401

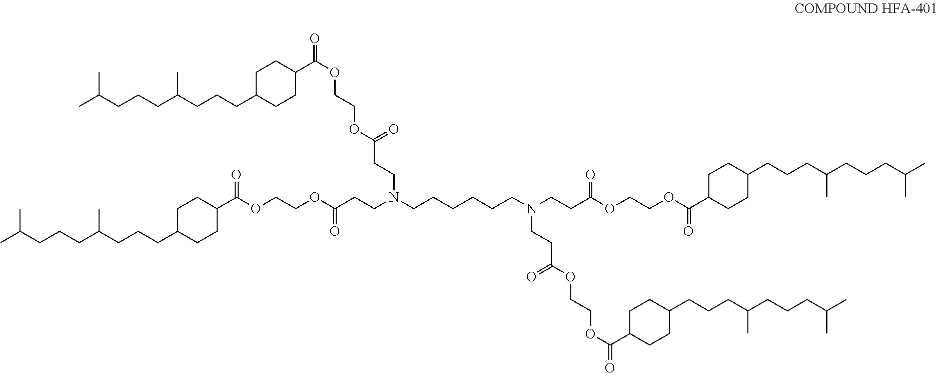 [SMILES: CC(C)CCCC(C)CCCC1CCC(C(=O)OCCOC(=O)CCN(CCCCCCN(CCC(=O)OCCOC(=O)C2CCC(CCCC(C)CCCC(C)C)CC2)CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-401 was prepared in accordance with the General Procedure D. The crude was purified by silica gel flash chromatography (eluent: 5% MeOH/DCM). ¹H NMR (400 MHz, Chloroform-d) δ 4.37-4.21 (m, 16H), 2.75 (t, J=7.2 Hz, 6H), 2.51-2.33 (m, 10H), 2.00-1.67 (m, 16H), 1.59-0.97 (m, 88H), 0.88-0.76 (m, 40H).

### Example 33—Preparation of Compound HFA-402

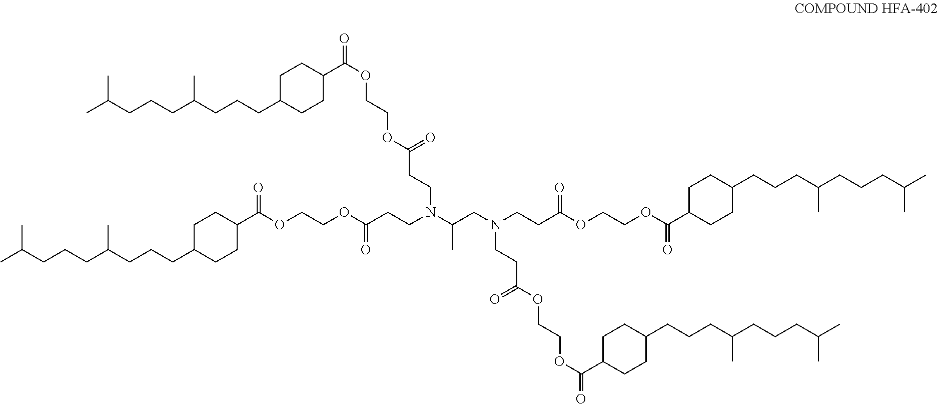 [SMILES: CC(C)CCCC(C)CCCC1CCC(C(=O)OCCOC(=O)CCN(CCC(=O)OCCOC(=O)C2CCC(CCCC(C)CCCC(C)C)CC2)CC(C)N(CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-402 was prepared in accordance with the General Procedure D. The crude was purified by silica gel flash chromatography (eluent: 5% MeOH/DCM). ¹H NMR (400 MHz, Chloroform-d) δ 4.39-4.13 (m, 16H), 2.51-2.28 (m, 8H), 2.00-1.73 (m, 14H), 1.58-0.96 (m, 88H), 0.83 (dd, J=10.4, 6.6 Hz, 44H).

### Example 34—Preparation of Compound HFA-403

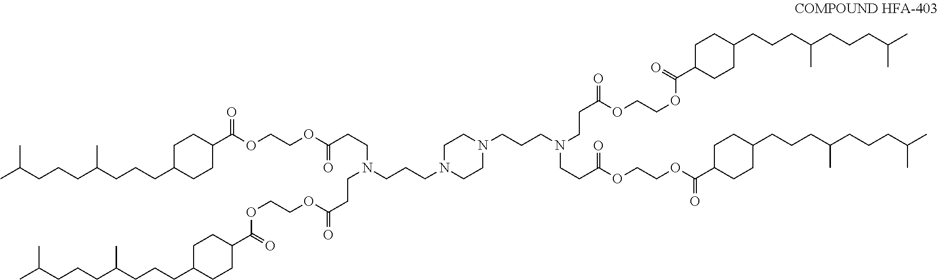 [SMILES: CC(C)CCCC(C)CCCC1CCC(C(=O)OCCOC(=O)CCN(CCCN2CCN(CCCN(CCC(=O)OCCOC(=O)C3CCC(CCCC(C)CCCC(C)C)CC3)CCC(=O)OCCOC(=O)C3CCC(CCCC(C)CCCC(C)C)CC3)CC2)CCC(=O)OCCOC(=O)C2CCC(CCCC(C)CCCC(C)C)CC2)CC1]

### ((3,3′,3″,3′″-((piperazine-1,4-diylbis(propane-3,1-diyl))bis(azanetriyl))tetrakis(propanoyl))tetrakis(oxy))tetrakis(ethane-2,1-diyl) tetrakis(4-(4,8-dimethylnonyl)cyclohexanecarboxylate)

Compound HFA-403 was prepared in accordance with the General Procedure D. The crude was purified by silica gel flash chromatography (eluent: 5% MeOH/DCM). ¹H NMR (400 MHz, Chloroform-d) δ 4.34-4.14 (m, 16H), 2.80-2.68 (m, 8H), 2.65-2.15 (m, 28H), 2.02-1.67 (m, 13H), 1.66-0.95 (m, 78H), 0.93-0.72 (m, 41H).

### Example 35—Preparation of Compound HFA-404

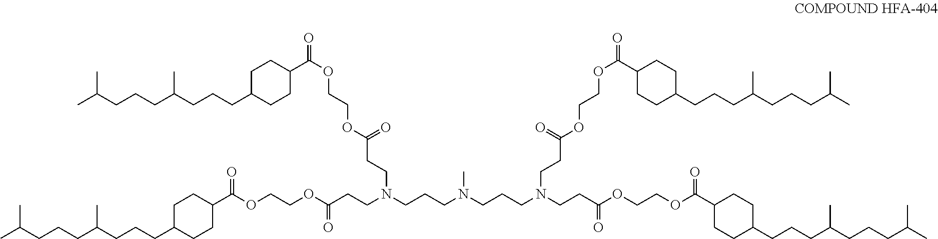 [SMILES: CC(C)CCCC(C)CCCC1CCC(C(=O)OCCOC(=O)CCN(CCCN(C)CCCN(CCC(=O)OCCOC(=O)C2CCC(CCCC(C)CCCC(C)C)CC2)CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-404 was prepared in accordance with the General Procedure D. The crude was purified by silica gel flash chromatography (eluent: 5% MeOH/DCM). ¹H NMR (400 MHz, Chloroform-d) δ 4.37-4.13 (m, 16H), 2.92-2.15 (m, 26H), 2.04-1.65 (m, 15H), 1.63-0.96 (m, 78H), 0.91-0.71 (m, 44H).

### Example 36—Preparation of Compound HFA-601

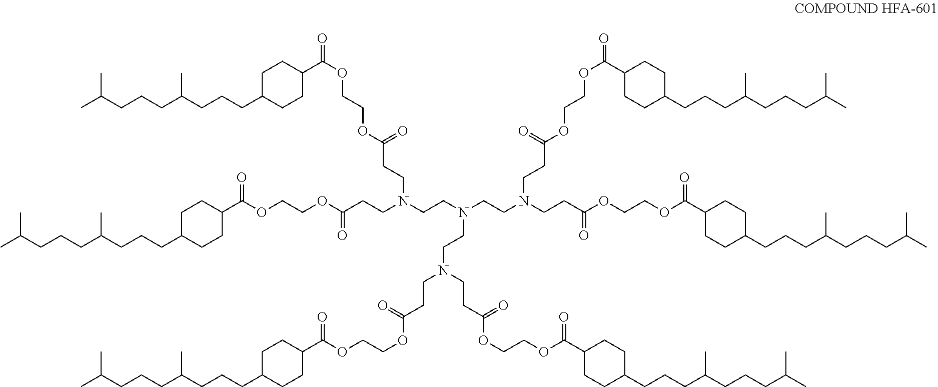 [SMILES: CC(C)CCCC(C)CCCC1CCC(C(=O)OCCOC(=O)CCN(CCC(=O)OCCOC(=O)C2CCC(CCCC(C)CCCC(C)C)CC2)CCN(CCN(CCC(=O)OCCOC(=O)C2CCC(CCCC(C)CCCC(C)C)CC2)CCC(=O)OCCOC(=O)C2CCC(CCCC(C)CCCC(C)C)CC2)CCN(CCC(=O)OCCOC(=O)C2CCC(CCCC(C)CCCC(C)C)CC2)CCC(=O)OCCOC(=O)C2CCC(CCCC(C)CCCC(C)C)CC2)CC1]

Compound HFA-601 was prepared in accordance with the General Procedure D. The crude was purified by silica gel flash chromatography (eluent: 10% MeOH/DCM). ¹H NMR (400 MHz, Chloroform-d) δ 4.31-4.13 (m, 24H), 2.76 (t, J=7.1 Hz, 10H), 2.66-2.37 (m, 22H), 2.00-1.66 (m, 21H), 1.61-0.94 (m, 118H), 0.91-0.72 (m, 63H).

### Example 37—Preparation of Compound FE (Diels-Alder Reaction, E)

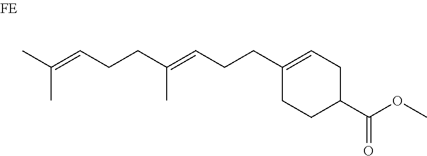 [SMILES: COC(=O)C1CC=C(CC/C=C(\C)CCC=C(C)C)CC1]

In a 4 mL sealed tube, farnesene (2.0 g, 9.8 mmol) is combined with methyl acrylate (0.93 g, 10.8 mmol). The tube was sealed and the reaction was stirred for 20 h at 130° C. The cooled reaction mixture was purified by silica gel flash column chromatography with 3% EtOAc/hexane eluants.

Colorless oil; 2.01 g, yield 72%;

1H NMR (400 MHz, Chloroform-d) δ 5.43-5.31 (m, 1H), 5.14-5.01 (m, 2H), 3.67-3.64 (m, 3H), 2.58-2.41 (m, 1H), 2.24-1.90 (m, 14H), 1.67-1.64 (m, 3H), 1.59-1.55 (m, 6H). 13C NMR (101 MHz, Chloroform-d) δ 176.56, 176.52, 137.39, 136.01, 135.17, 135.14, 131.30, 124.41, 124.40, 124.11, 124.08, 120.36, 118.96, 51.67, 51.64, 39.87, 39.78, 39.43, 37.75, 37.57, 30.72, 27.76, 27.75, 26.79, 26.30, 26.29, 25.76, 25.61, 25.21, 24.63, 17.75, 16.07, 16.06.

### Example 38—Preparation of Compound ME (Diels-Alder Reaction, E)

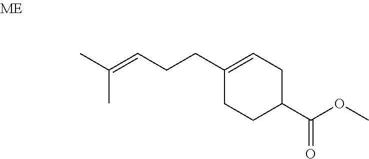 [SMILES: COC(=O)C1CC=C(CCC=C(C)C)CC1]

In a 4 mL sealed tube, myrcene (2.0 g, 14.7 mmol) is combined with methyl acrylate (1.4 g, 16.1 mmol). The tube was sealed and the reaction was stirred for 20 h at 130° C. The cooled reaction mixture was purified by silica gel flash column chromatography with 5% EtOAc/hexane eluants.

Colorless oil; 2.6 g, yield 72%;

1H NMR (400 MHz, Chloroform-d) δ 5.40-5.32 (m, 1H), 5.10-5.01 (m, 1H), 3.67-3.62 (m, 3H), 2.57-2.40 (m, 1H), 2.24-1.86 (m, 9H), 1.70-1.58 (m, 4H), 1.56 (s, 3H). 13C NMR (101 MHz, Chloroform-d) δ 176.51, 176.47, 137.38, 136.02, 131.51, 131.47, 124.21, 124.18, 120.29, 118.90, 51.63, 51.61, 39.84, 39.40, 37.75, 37.58, 30.70, 27.73, 26.41, 26.39, 25.74, 25.58, 25.19, 24.61, 17.73, 17.72.

### Example 39—Preparation of Compound FE-211 (Transesterification Reaction, G)

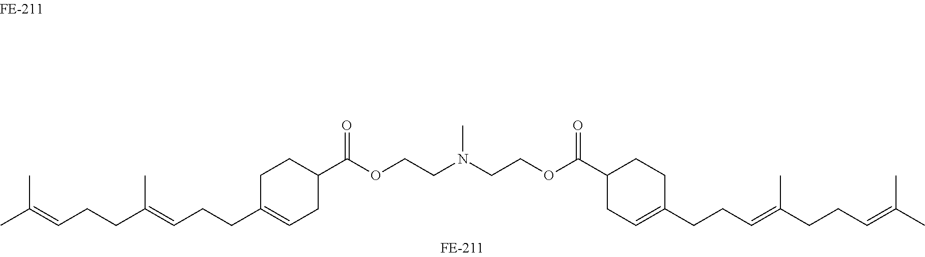 [SMILES: CC(C)=CCC/C(C)=C/CCC1=CCC(C(=O)OCCN(C)CCOC(=O)C2CC=C(CC/C=C(\C)CCC=C(C)C)CC2)CC1]

In a 4 mL glass tube, FE (304 mg, 1.0 mmol) is combined with an amine 211 (50 mg, 0.4 mmol) and triazabicyclodecene (TBD) (24 mg, 0.2 mmol). The tube was sealed and the reaction mixture was stirred for 16 h at 130° C. The cooled reaction mixture was purified by silica gel flash column chromatography with 20% EtOAc/hexane.

Colorless oil; 170 mg, yield 64%;

1H NMR (400 MHz, Chloroform-d) δ 5.35 (s, 2H), 5.07 (s, 4H), 4.16 (t, J=5.7 Hz, 4H), 2.70 (t, J=5.7 Hz, 4H), 2.58-2.40 (m, 2H), 2.33 (s, 3H), 2.25-1.85 (m, 27H), 1.68-1.61 (m, 7H), 1.57 (s, 12H). 13C NMR (101 MHz, Chloroform-d) δ 175.91, 175.89, 137.36, 135.92, 135.11, 135.08, 131.23, 124.41, 124.37, 124.07, 124.04, 120.29, 118.88, 62.03, 61.99, 55.91, 42.87, 42.84, 39.86, 39.74, 39.43, 37.71, 37.55, 30.65, 27.71, 26.75, 26.28, 26.24, 25.74, 25.55, 25.17, 24.57, 17.73, 16.05, 16.03.

### Example 40—Preparation of Compound FE-212 (Transesterification Reaction, G)

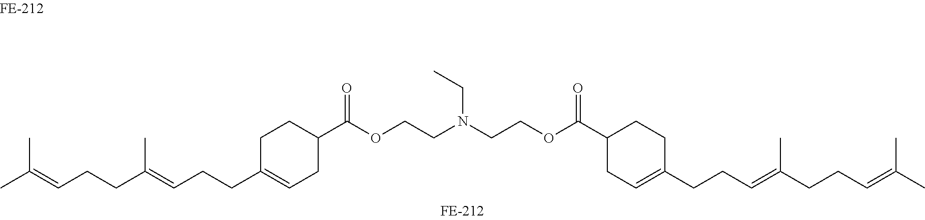 [SMILES: CCN(CCOC(=O)C1CC=C(CC/C=C(\C)CCC=C(C)C)CC1)CCOC(=O)C1CC=C(CC/C=C(\C)CCC=C(C)C)CC1]

In a 4 mL glass tube, FE (272 mg, 0.9 mmol) is combined with an amine 212 (50 mg, 0.4 mmol) and triazabicyclodecene (TBD) (11 mg, 0.1 mmol). The tube was sealed and the reaction mixture was stirred for 16 h at 130° C. The cooled reaction mixture was purified by silica gel flash column chromatography with 20% EtOAc/hexane.

Colorless oil; 144 mg, yield 59%; 1H NMR (400 MHz, Chloroform-d) δ 5.41-5.33 (m, 2H), 5.13-5.02 (m, 4H), 4.17-4.08 (m, 4H), 2.79-2.72 (m, 4H), 2.61 (qd, J=7.1, 1.8 Hz, 2H), 2.56-2.41 (m, 2H), 2.25-2.11 (m, 4H), 2.08-1.90 (m, 23H), 1.69-1.62 (m, 7H), 1.59-1.54 (m, 12H), 1.01 (td, J=7.1, 1.5 Hz, 3H). 13C NMR (101 MHz, Chloroform-d) δ 175.99, 175.96, 137.42, 136.00, 135.17, 135.14, 131.31, 124.41, 124.12, 124.08, 120.34, 118.94, 62.59, 62.55, 52.32, 48.70, 48.68, 39.95, 39.78, 39.52, 37.76, 37.59, 30.71, 27.77, 27.75, 26.79, 26.33, 26.29, 25.79, 25.60, 25.21, 24.64, 17.77, 16.09, 16.07, 12.26.

### Example 41—Preparation of Compound FE-213 (Transesterification Reaction, G)

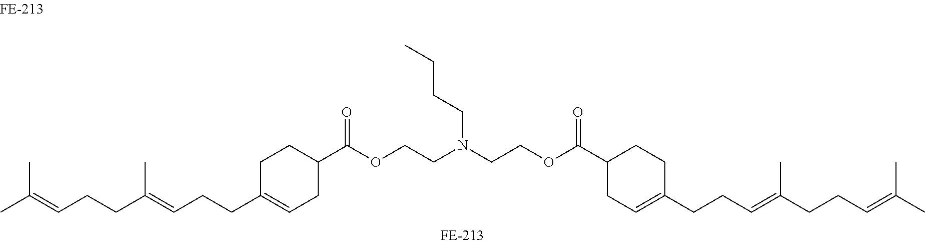 [SMILES: CCCCN(CCOC(=O)C1CC=C(CC/C=C(\C)CCC=C(C)C)CC1)CCOC(=O)C1CC=C(CC/C=C(\C)CCC=C(C)C)CC1]

In a 4 mL glass tube, FE (225 mg, 0.8 mmol) is combined with an amine 213 (50 mg, 0.3 mmol) and triazabicyclodecene (TBD) (9 mg, 0.1 mmol). The tube was sealed and the reaction mixture was stirred for 16 h at 130° C. The cooled reaction mixture was purified by silica gel flash column chromatography with 15% EtOAc/hexane.

Colorless oil; 178 mg, yield 85%; 1H NMR (400 MHz, Chloroform-d) δ 5.36 (s, 2H), 5.13-5.02 (m, 4H), 4.17-4.06 (m, 4H), 2.74 (t, J=6.0 Hz, 4H), 2.58-2.38 (m, 4H), 2.24-1.87 (m, 27H), 1.69-1.61 (m, 7H), 1.60-1.53 (m, 12H), 1.44-1.34 (m, 2H), 1.33-1.22 (m, 2H), 0.88 (t, J=7.3 Hz, 3H). 13C NMR (101 MHz, Chloroform-d) δ 175.97, 175.94, 137.39, 135.99, 135.14, 135.11, 131.26, 124.40, 124.10, 124.07, 120.33, 118.94, 62.61, 62.58, 54.83, 54.81, 52.90, 39.96, 39.77, 39.52, 37.75, 37.58, 30.70, 29.71, 29.70, 27.76, 27.74, 26.78, 26.32, 26.28, 25.77, 25.59, 25.20, 24.63, 20.45, 17.75, 16.07, 16.06, 14.11.

### Example 42—Preparation of Compound FE-214 (Transesterification Reaction, G)

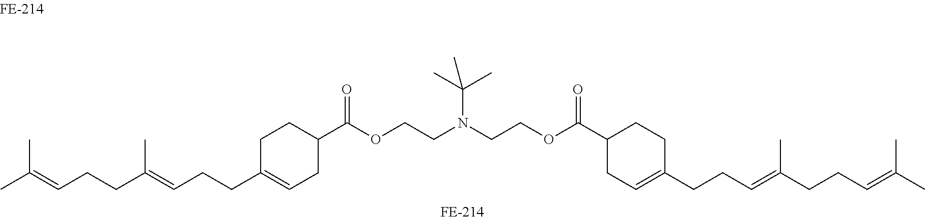 [SMILES: CC(C)=CCC/C(C)=C/CCC1=CCC(C(=O)OCCN(CCOC(=O)C2CC=C(CC/C=C(\C)CCC=C(C)C)CC2)C(C)(C)C)CC1]

In a 4 mL glass tube, FE (225 mg, 0.8 mmol) is combined with an amine 214 (50 mg, 0.3 mmol) and triazabicyclodecene (TBD) (9 mg, 0.1 mmol). The tube was sealed and the reaction mixture was stirred for 16 h at 130° C. The cooled reaction mixture was purified by silica gel flash column chromatography with 15% EtOAc/hexane.

Colorless oil; 144 mg, yield 69%; 1H NMR (400 MHz, Chloroform-d) δ 5.41-5.34 (m, 21H), 5.12-5.04 (m, 4H), 4.05 (td, J=6.8, 2.0 Hz, 4H), 2.75 (t, J=6.9 Hz, 4H), 2.59-2.40 (m, 2H), 2.26-1.88 (m, 27H), 1.72-1.61 (m, 7H), 1.61-1.54 (m, 12H), 1.05 (s, 9H). 13C NMR (101 MHz, Chloroform-d) δ 176.05, 176.02, 137.41, 136.07, 135.17, 135.15, 131.32, 131.31, 124.44, 124.43, 124.15, 124.11, 120.35, 119.01, 64.93, 54.87, 49.56, 49.53, 40.00, 39.80, 39.56, 37.79, 37.61, 30.77, 27.82, 27.80, 27.23, 26.81, 26.35, 26.32, 25.80, 25.66, 25.27, 24.69, 17.79, 16.11, 16.09.

### Example 43—Preparation of Compound FE-415 (Transesterification Reaction, G)

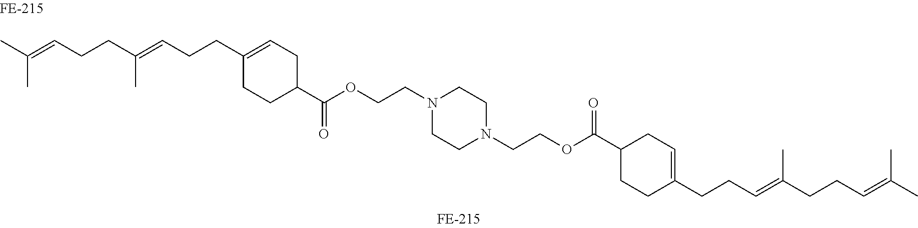 [SMILES: CC(C)=CCC/C(C)=C/CCC1=CCC(C(=O)OCCN2CCN(CCOC(=O)C3CC=C(CC/C=C(\C)CCC=C(C)C)CC3)CC2)CC1]

In a 4 mL glass tube, FE (417 mg, 1.4 mmol) is combined with an amine 215 (100 mg, 0.6 mmol) and triazabicyclodecene (TBD) (40 mg, 0.3 mmol). The tube was sealed and the reaction mixture was stirred for 16 h at 130° C. The cooled reaction mixture was purified by silica gel flash column chromatography with 50% EtOAc/hexane.

Brown oil; 200 mg, yield 50%; 1H NMR (400 MHz, Chloroform-d) δ 5.35 (s, 2H), 5.06 (s, 4H), 4.18 (t, J=5.6 Hz, 4H), 2.66-2.36 (m, 13H), 2.27-1.76 (m, 27H), 1.71-1.37 (m, 20H). 13C NMR (101 MHz, Chloroform-d) δ 175.79, 175.76, 137.30, 135.87, 135.04, 135.02, 131.16, 124.33, 124.02, 124.00, 120.26, 118.86, 61.67, 56.63, 53.26, 39.80, 39.69, 39.38, 37.67, 37.50, 30.61, 27.67, 27.65, 26.70, 26.23, 26.20, 25.71, 25.51, 25.13, 24.50, 17.69, 16.02, 16.00.

### Example 44—Preparation of Compound FE-301 (Transesterification Reaction, (G)

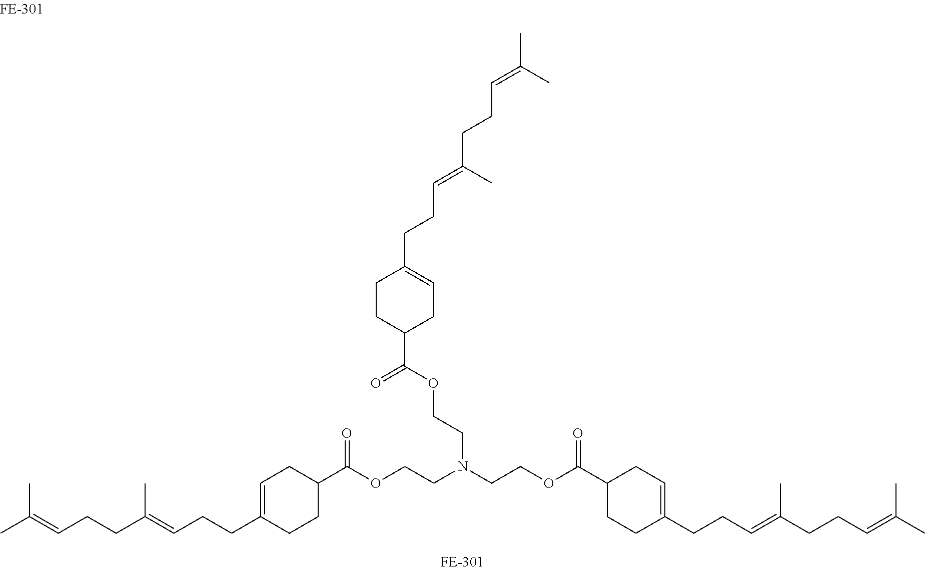 [SMILES: CC(C)=CCC/C(C)=C/CCC1=CCC(C(=O)OCCN(CCOC(=O)C2CC=C(CC/C=C(\C)CCC=C(C)C)CC2)CCOC(=O)C2CC=C(CC/C=C(\C)CCC=C(C)C)CC2)CC1]

In a 4 mL glass tube, FE (343 mg, 1.2 mmol) is combined with an amine 301 (50 mg, 0.3 mmol) and triazabicyclodecene (TBD) (28 mg, 0.2 mmol). The tube was sealed and the reaction mixture was stirred for 16 h at 130° C. The cooled reaction mixture was purified by silica gel flash column chromatography with 20% EtOAc/hexane.

Pale-brown oil; 110 mg, yield 35%; 1H NMR (400 MHz, Chloroform-d) δ 5.36 (s, 3H), 5.14-4.98 (m, 6H), 4.12 (t, J=5.2 Hz, 6H), 2.82 (s, 6H), 2.57-2.39 (m, 3H), 2.28-1.81 (m, 40H), 1.69-1.51 (m, 29H). 13C NMR (101 MHz, Chloroform-d) δ 175.92, 175.89, 137.45, 135.96, 135.17, 135.15, 131.30, 124.41, 124.10, 124.07, 120.32, 118.88, 62.56, 53.33, 39.94, 39.78, 39.51, 37.75, 37.59, 30.70, 27.78, 27.75, 26.79, 26.33, 26.28, 25.79, 25.59, 25.22, 24.63, 17.77, 16.09, 16.07.

### Example 45—Preparation of Compound FE-301-2 (Transesterification Reaction, G)

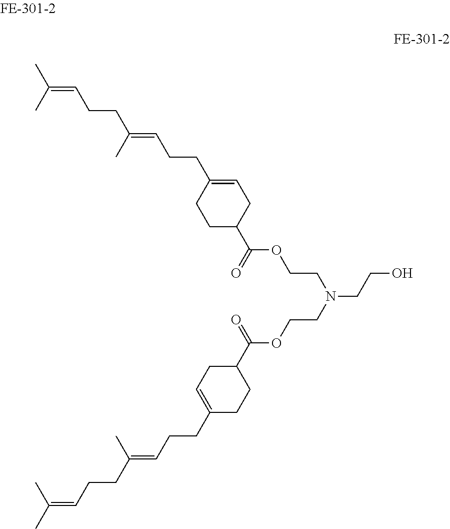 [SMILES: CC(C)=CCC/C(C)=C/CCC1=CCC(C(=O)OCCN(CCO)CCOC(=O)C2CC=C(CC/C=C(\C)CCC=C(C)C)CC2)CC1]

In the above reaction, a polar compound was isolated with 40% EtOAc/hexane eluant which turned out to be FE-301-2.

Pale-brown oil; 145 mg, yield 65%; 1H NMR (400 MHz, Chloroform-d) δ 5.36 (s, 2H), 5.14-5.00 (m, 4H), 4.14 (s, 4H), 3.53 (t, J=4.7 Hz, 3H), 2.83 (s, 4H), 2.72 (s, 2H), 2.61-2.41 (m, 2H), 2.25-1.88 (m, 26H), 1.69-1.51 (m, 20H). 13C NMR (101 MHz, Chloroform-d) δ 176.05, 176.02, 137.46, 135.90, 135.17, 135.15, 131.30, 124.40, 124.09, 124.06, 120.31, 118.81, 62.17, 62.13, 58.83, 56.39, 56.35, 52.98, 39.94, 39.77, 39.51, 37.72, 37.57, 30.68, 27.75, 27.73, 26.78, 26.32, 26.25, 25.78, 25.54, 25.18, 24.60, 17.76, 16.08, 16.06.

### Example 46—Preparation of Compound ME-211 (Transesterification Reaction, G)

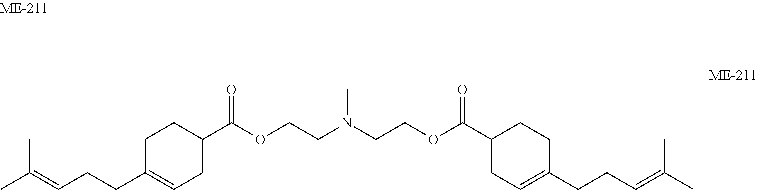 [SMILES: CC(C)=CCCC1=CCC(C(=O)OCCN(C)CCOC(=O)C2CC=C(CCC=C(C)C)CC2)CC1]

In a 4 mL glass tube, ME (233 mg, 1.1 mmol) is combined with an amine 211 (50 mg, 0.4 mmol) and triazabicyclodecene (TBD) (24 mg, 0.2 mmol). The tube was sealed and the reaction mixture was stirred for 16 h at 130° C. The cooled reaction mixture was purified by silica gel flash column chromatography with 20% EtOAc/hexane.

Colorless oil; 155 mg, yield 72%; 1H NMR (400 MHz, Chloroform-d) δ 5.33 (s, 2H), 5.08-4.98 (m, 2H), 4.21-4.05 (m, 4H), 2.69 (t, J 5.7 Hz, 4H), 2.56-2.39 (m, 2H), 2.32 (d, J=1.4 Hz, 3H), 2.22-1.86 (m, 19H), 1.66-1.60 (m, 7H), 1.55 (s, 6H). 13C NMR (101 MHz, Chloroform-d) δ 175.86, 175.84, 137.33, 135.91, 131.43, 131.40, 124.15, 124.13, 120.20, 118.80, 61.90, 61.86, 55.79, 55.77, 42.73, 42.72, 39.81, 39.38, 37.69, 37.53, 30.62, 27.66, 26.34, 25.71, 25.49, 25.12, 24.53, 17.71, 17.69.

### Example 47—Preparation of Compound ME-212 (Transesterification Reaction, G)

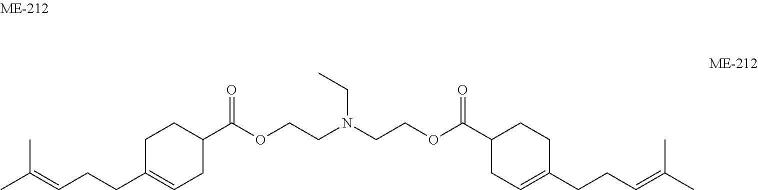 [SMILES: CCN(CCOC(=O)C1CC=C(CCC=C(C)C)CC1)CCOC(=O)C1CC=C(CCC=C(C)C)CC1]

In a 4 mL glass tube, ME (314 mg, 1.4 mmol) is combined with an amine 212 (75 mg, 0.6 mmol) and triazabicyclodecene (TBD) (40 mg, 0.1 mmol). The tube was sealed and the reaction mixture was stirred for 16 h at 130° C. The cooled reaction mixture was purified by silica gel flash column chromatography with 20% EtOAc/hexane.

Colorless oil; 174 mg, yield 60%; 1H NMR (400 MHz, Chloroform-d) δ 5.40-5.30 (m, 2H), 5.12-4.98 (m, 2H), 4.16-4.05 (m, 4H), 2.78-2.69 (m, 4H), 2.59 (qd, J=7.1, 1.8 Hz, 2H), 2.55-2.39 (m, 2H), 2.25-1.85 (m, 18H), 1.71-1.48 (m, 14H), 0.99 (td, J=7.1, 1.4 Hz, 3H). 13C NMR (101 MHz, Chloroform-d) δ 175.92, 175.89, 137.36, 135.98, 131.46, 131.44, 124.19, 124.17, 120.24, 118.87, 62.61, 62.57, 52.35, 48.69, 48.68, 39.90, 39.46, 37.73, 37.56, 30.68, 27.71, 26.38, 25.74, 25.54, 25.16, 24.59, 17.73, 17.72, 12.29.

### Example 48—Preparation of Compound ME-213 (Transesterification Reaction, G)

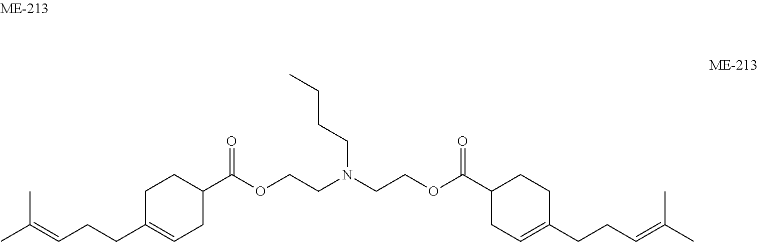 [SMILES: CCCCN(CCOC(=O)C1CC=C(CCC=C(C)C)CC1)CCOC(=O)C1CC=C(CCC=C(C)C)CC1]

In a 4 mL glass tube, ME (259 mg, 1.2 mmol) is combined with an amine 213 (75 mg, 0.5 mmol) and triazabicyclodecene (TBD) (13 mg, 0.1 mmol). The tube was sealed and the reaction mixture was stirred for 16 h at 130° C. The cooled reaction mixture was purified by silica gel flash column chromatography with 10% EtOAc/hexane.

Colorless oil; 156 mg, yield 62%; 1H NMR (400 MHz, Chloroform-d) δ 5.40-5.29 (m, 2H), 5.12-4.97 (m, 2H), 4.16-4.05 (m, 4H), 2.74 (t, J=6.0 Hz, 4H), 2.55-2.40 (m, 4H), 2.26-1.87 (m, 19H), 1.68-1.60 (m, 7H), 1.56 (s, 6H), 1.43-1.34 (m, 2H), 1.32-1.21 (m, 2H), 0.87 (t, J=7.3 Hz, 3H). 13C NMR (101 MHz, Chloroform-d) δ 175.95, 175.93, 137.39, 136.01, 131.50, 131.47, 124.21, 124.18, 120.27, 118.89, 62.58, 62.54, 54.82, 54.80, 52.90, 39.94, 39.50, 37.75, 37.58, 30.70, 29.67, 29.66, 27.74, 27.72, 26.40, 25.76, 25.56, 25.19, 24.62, 20.44, 17.76, 17.74, 14.09.

### Example 49—Preparation of Compound ME-214 (Transesterification Reaction, G)

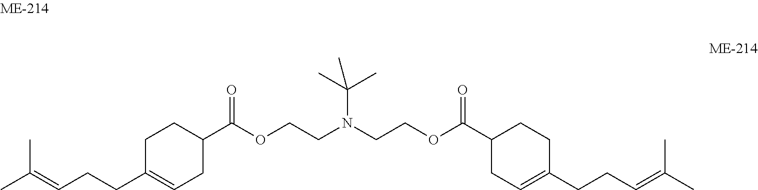 [SMILES: CC(C)=CCCC1=CCC(C(=O)OCCN(CCOC(=O)C2CC=C(CCC=C(C)C)CC2)C(C)(C)C)CC1]

In a 4 mL glass tube, ME (259 mg, 1.2 mmol) is combined with an amine 214 (75 mg, 0.5 mmol) and triazabicyclodecene (TBD) (13 mg, 0.1 mmol). The tube was sealed and the reaction mixture was stirred for 16 h at 130° C. The cooled reaction mixture was purified by silica gel flash column chromatography with 10% EtOAc/hexane.

Colorless oil; 163 mg, yield 65%; 1H NMR (400 MHz, Chloroform-d) δ 5.42-5.30 (m, 2H), 5.13-5.00 (m, 2H), 4.04 (t, J=6.9 Hz, 4H), 2.75 (t, J=6.9 Hz, 4H), 2.61-2.36 (m, 2H), 2.24-1.88 (m, 18H), 1.75-1.48 (m, 14H), 1.05 (s, 9H). 13C NMR (101 MHz, Chloroform-d) δ 176.05, 176.02, 137.42, 136.10, 131.56, 131.53, 124.25, 124.23, 120.30, 118.96, 64.94, 54.88, 49.56, 39.99, 39.55, 37.79, 37.62, 30.77, 27.80, 27.79, 27.24, 26.44, 25.80, 25.64, 25.26, 24.68, 17.80, 17.78.

### Example 50—Preparation of Compound ME-215 (Transesterification Reaction, G)

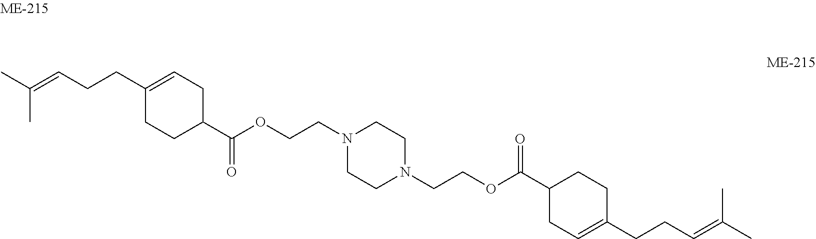 [SMILES: CC(C)=CCCC1=CCC(C(=O)OCCN2CCN(CCOC(=O)C3CC=C(CCC=C(C)C)CC3)CC2)CC1]

In a 4 mL glass tube, ME (239 mg, 1.1 mmol) is combined with an amine 215 (75 mg, 0.4 mmol) and triazabicyclodecene (TBD) (12 mg, 0.1 mmol). The tube was sealed and the reaction mixture was stirred for 16 h at 130° C. The cooled reaction mixture was purified by silica gel flash column chromatography with 40% EtOAc/hexane.

Colorless oil; 200 mg, yield 84%; 1H NMR (400 MHz, Chloroform-d) δ 5.32 (s, 2H), 5.11-4.95 (m, 2H), 4.16 (t, J=5.7 Hz, 4H), 2.63-2.36 (m, 13H), 2.21-1.82 (m, 19H), 1.70-1.48 (m, 14H). 13C NMR (101 MHz, Chloroform-d) δ 175.81, 175.78, 137.32, 135.90, 131.42, 131.39, 124.14, 124.12, 120.21, 118.80, 61.65, 56.60, 53.20, 39.79, 39.36, 37.68, 37.52, 30.62, 27.66, 27.63, 26.33, 25.71, 25.49, 25.11, 24.49, 17.71, 17.69.

### Example 51—Preparation of Compound ME-301 (Transesterification Reaction, G)

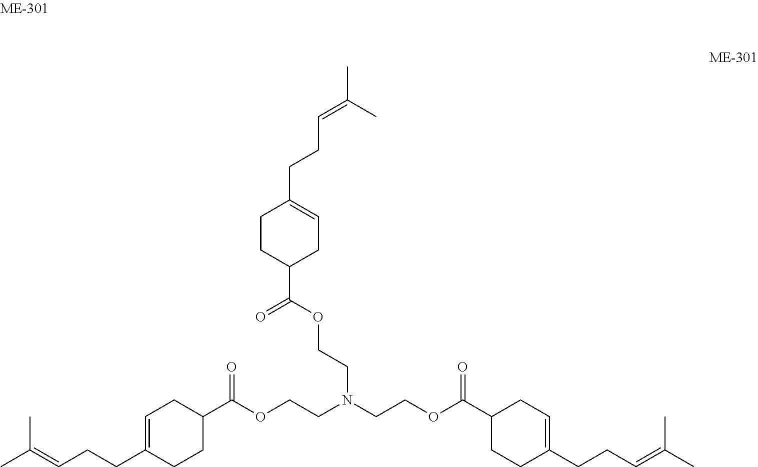 [SMILES: CC(C)=CCCC1=CCC(C(=O)OCCN(CCOC(=O)C2CC=C(CCC=C(C)C)CC2)CCOC(=O)C2CC=C(CCC=C(C)C)CC2)CC1]

In a 4 mL glass tube, ME (260 mg, 1.2 mmol) is combined with an amine 301 (50 mg, 0.3 mmol) and triazabicyclodecene (TBD) (28 mg, 0.2 mmol). The tube was sealed and the reaction mixture was stirred for 16 h at 130° C. The cooled reaction mixture was purified by silica gel flash column chromatography with 20% EtOAc/hexane.

Pale-brown oil; 118 mg, yield 49%; 1H NMR (400 MHz, Chloroform-d) δ 5.43-5.31 (m, 3H), 5.12-5.00 (m, 3H), 4.14 (s, 6H), 2.85 (s, 6H), 2.58-2.40 (m, 3H), 2.24-1.88 (m, 29H), 1.68-1.64 (m, 10H), 1.58 (s, 9H). 13C NMR (101 MHz, Chloroform-d) δ 175.94, 175.91, 137.49, 136.00, 131.60, 131.58, 124.23, 124.20, 120.30, 118.84, 62.47, 53.31, 39.95, 39.52, 37.77, 37.61, 30.73, 27.78, 27.76, 26.44, 26.42, 25.81, 25.58, 25.22, 24.64, 17.81, 17.79.

### Example 52—Preparation of Compound ME-301-2 (Transesterification Reaction, G)

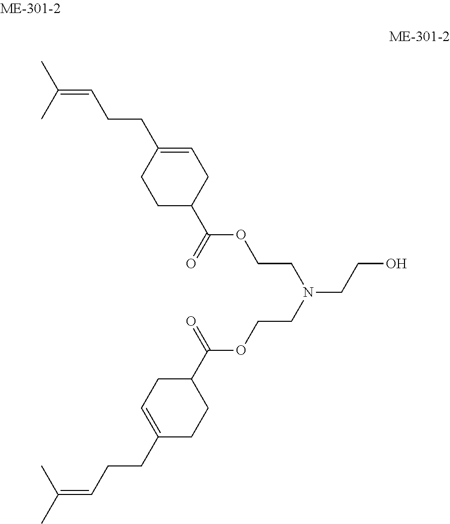 [SMILES: CC(C)=CCCC1=CCC(C(=O)OCCN(CCO)CCOC(=O)C2CC=C(CCC=C(C)C)CC2)CC1]

In the above reaction, a polar compound was isolated with 40% EtOAc/hexane eluant which turned out to be ME-301-2.

Pale-brown oil; 66 mg, yield 37%; 1H NMR (400 MHz, Chloroform-d) δ 5.42-5.33 (m, 2H), 5.14-5.00 (m, 2H), 4.24-4.07 (m, 4H), 3.56 (t, J=5.1 Hz, 2H), 2.86 (t, J=5.5 Hz, 4H), 2.75 (t, J=4.6 Hz, 2H), 2.61-2.42 (m, 2H), 2.25-1.91 (m, 19H), 1.68-1.55 (m, 14H). 13C NMR (101 MHz, Chloroform-d) δ 176.10, 176.07, 137.53, 135.98, 131.65, 131.63, 124.25, 124.22, 120.32, 118.81, 62.12, 62.07, 58.80, 58.79, 56.48, 56.45, 53.05, 53.03, 39.98, 39.56, 37.78, 37.63, 30.74, 27.78, 26.46, 26.43, 25.83, 25.58, 25.22, 24.64, 17.83, 17.82.

### Example 53—Preparation of Compound FE-216 (Synthesis of FE-216 and ME-216 Lipidoids H)

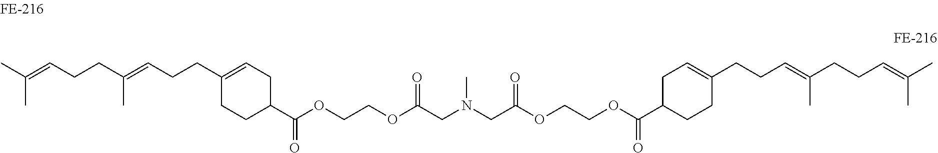 [SMILES: CC(C)=CCC/C(C)=C/CCC1=CCC(C(=O)OCCOC(=O)CN(C)CC(=O)OCCOC(=O)C2CC=C(CC/C=C(\C)CCC=C(C)C)CC2)CC1]

In a 20 mL scintillation glass vial, methyliminodiacetic acid (216) (50 mg, 0.3 mmol, 1.0 eq), 1-ethyl-3-(3-dimethylamino propyl)carbodiimide (EDC-HCl) (143 mg, 0.7 mmol, 2.2 eq), and dimethyl aminopyridine (DMAP) (91 mg, 0.7 mmol, 2.2 eq) were dissolved in dichloromethane and dimethylformamide (1:1, 5 mL). The mixture was stirred for 15 minutes at room temperature before adding Compound 1 (326 mg, 1.0 mmol, 2.2 eq) dissolved in dichloromethane (1.0 mL). The reaction was then stirred for 20 h at room temperature. The reaction mixture was transferred to a separatory funnel, added 20 mL of distilled water, and extracted with dichloromethane (3×40 mL). Combined organic extracts were dried over Na2SO4, filtered to a round bottom flask and the solvent was evaporated under reduced pressure. The crude residue was purified by silica gel flash column chromatography with 20% EtOAc/hexane eluant.

Colorless oil; 137 mg, yield 53%; 1H NMR (400 MHz, Chloroform-d) δ 5.42-5.29 (m, 2H), 5.16-4.96 (m, 4H), 4.39-4.12 (m, 8H), 3.50 (s, 4H), 2.61-2.33 (m, 5H), 2.28-1.70 (m, 27H), 1.68-1.48 (m, 19H). 13C NMR (101 MHz, Chloroform-d) δ 175.69, 175.65, 170.50, 137.38, 135.82, 135.14, 135.11, 131.24, 124.34, 124.02, 123.98, 120.28, 118.75, 62.30, 61.86, 56.89, 42.04, 39.72, 39.31, 37.67, 37.51, 30.53, 27.63, 27.61, 26.73, 26.25, 26.21, 25.73, 25.49, 25.11, 24.48, 17.71, 16.04, 16.02.

### Example 54—Preparation of Compound ME-216 (Synthesis of FE-216 and ME-216 Lipidoids H)

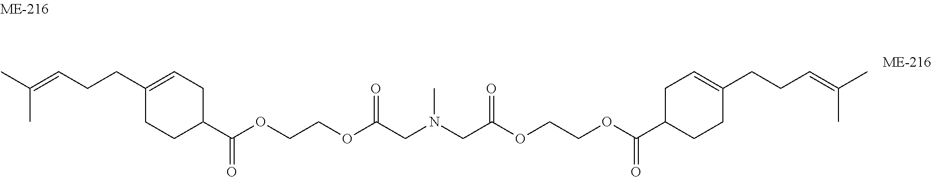 [SMILES: CC(C)=CCCC1=CCC(C(=O)OCCOC(=O)CN(C)CC(=O)OCCOC(=O)C2CC=C(CCC=C(C)C)CC2)CC1]

In a 20 mL scintillation glass vial, methyliminodiacetic acid (216) (50 mg, 0.3 mmol, 1.0 eq), 1-ethyl-3-(3-dimethylamino propyl)carbodiimide (EDC-HCl) (143 mg, 0.7 mmol, 2.2 eq), and dimethyl aminopyridine (DMAP) (91 mg, 0.7 mmol, 2.2 eq) were dissolved in dichloromethane and dimethylformamide (1:1, 5 mL). The mixture was stirred for 15 minutes at room temperature before adding Compound 3 (257 mg, 1.0 mmol, 2.2 eq) dissolved in dichloromethane (1.0 mL). The reaction was then stirred for 20 h at room temperature. The reaction mixture was transferred to a separatory funnel, added 20 mL of distilled water, and extracted with dichloromethane (3×40 mL). Combined organic extracts were dried over Na₂SO₄, filtered to a round bottom flask and the solvent was evaporated under reduced pressure. The crude residue was purified by silica gel flash column chromatography with 20% EtOAc/hexane eluant.

Colorless oil; 160 mg, yield 77%; 1H NMR (400 MHz, Chloroform-d) δ 5.41-5.27 (m, 2H), 5.07-4.94 (m, 2H), 4.32-4.11 (m, 8H), 3.47 (s, 4H), 2.56-2.29 (m, 5H), 2.23-1.69 (m, 19H), 1.67-1.56 (m, 7H), 1.53 (s, 6H). 13C NMR (101 MHz, Chloroform-d) δ 175.60, 175.55, 170.44, 137.30, 135.78, 131.42, 131.38, 124.07, 124.05, 120.17, 118.66, 62.23, 61.84, 61.81, 56.83, 41.96, 39.65, 39.23, 37.61, 37.45, 30.48, 27.54, 26.28, 25.66, 25.40, 25.02, 24.40, 17.67, 17.65.

### Example 55—Preparation of Compound FE-211-OH (Mono-Transesterification Reaction, I)

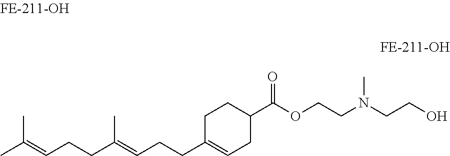 [SMILES: CC(C)=CCC/C(C)=C/CCC1=CCC(C(=O)OCCN(C)CCO)CC1]

In a 4 mL glass tube, FE (304 mg, 1.0 mmol) is combined with an amine 211 (50 mg, 0.4 mmol). The tube was sealed and the reaction mixture was stirred for 20 h at 130° C. The cooled reaction mixture was purified by silica gel flash column chromatography with 5% MeOH/DCM.

Colorless oil; 60 mg, yield 31%; 1H NMR (400 MHz, Chloroform-d) δ 5.42-5.30 (m, 1H), 5.14-4.99 (m, 2H), 4.24-4.09 (m, 2H), 3.56 (t, J=5.3 Hz, 2H), 3.21 (s, 1H), 2.71 (t, J=11.1 Hz, 2H), 2.59 (t, J=5.3 Hz, 2H), 2.55-2.41 (m, 1H), 2.32 (s, 3H), 2.25-1.87 (m, 14H), 1.65 (s, 3H), 1.57 (s, 6H).

### Example 56—Preparation of Compound FE-211-Oleic (Synthesis of Hybrid Lipidoids, J)

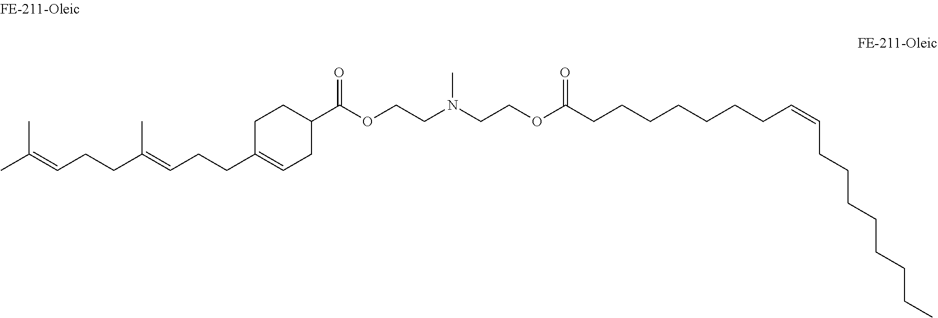 [SMILES: CCCCCCCC/C=C\CCCCCCCC(=O)OCCN(C)CCOC(=O)C1CC=C(CC/C=C(\C)CCC=C(C)C)CC1]

In a 20 mL scintillation glass vial, Oleic acid (50 mg, 0.2 mmol, 1.0 eq), 1-ethyl-3-(3-dimethyl aminopropyl)carbodiimide (EDC-HCl) (37 mg, 0.2 mmol, 1.1 eq), and dimethyl aminopyridine (DMAP) (24 mg, 0.2 mmol, 1.1 eq) were dissolved in dichloromethane (1.0 mL). The mixture was stirred for 15 minutes at room temperature before adding FE-211-OH (73 mg, 0.2 mmol, 1.1 eq) dissolved in dichloromethane (0.5 mL). The reaction was then stirred for 20 h at room temperature. The reaction mixture was transferred to a separatory funnel, added 20 mL of distilled water, and extracted with dichloromethane (3×40 mL). Combined organic extracts were dried over Na2SO4, filtered to a round bottom flask and the solvent was evaporated under reduced pressure. The crude residue was purified by silica gel flash column chromatography with 20% EtOAc/hexane eluant.

Colorless oil; 91 mg, yield 80%; 1H NMR (400 MHz, Chloroform-d) δ 5.41-5.25 (m, 3H), 5.06 (s, 2H), 4.25-4.06 (m, 4H), 2.79-2.63 (m, 4H), 2.59-2.41 (m, 1H), 2.34 (s, 3H), 2.28 (t, J=7.6 Hz, 2H), 2.24-2.11 (m, 2H), 2.10-1.90 (m, 151H), 1.69-1.53 (m, 12H), 1.38-1.17 (m, 20H), 0.91-0.80 (m, 3H). 13C NMR (101 MHz, Chloroform-d) δ 175.98, 175.95, 173.82, 137.43, 135.98, 135.19, 135.16, 131.31, 130.05, 129.79, 124.41, 124.11, 124.07, 120.33, 118.91, 61.95, 61.92, 61.80, 55.84, 42.80, 42.79, 39.91, 39.79, 39.48, 37.76, 37.59, 34.32, 32.00, 30.69, 29.85, 29.78, 29.62, 29.41, 29.27, 29.24, 29.21, 29.20, 27.76, 27.74, 27.30, 27.25, 26.80, 26.33, 26.29, 25.79, 25.59, 25.21, 24.98, 24.62, 22.78, 17.77, 16.08, 14.22.

### Example 57—Preparation of Compound HFE (Hydrogenation Reaction, F)

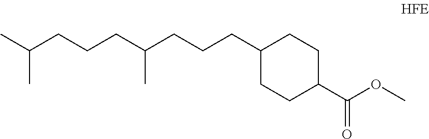 [SMILES: COC(=O)C1CCC(CCCC(C)CCCC(C)C)CC1]

In a 100 mL round-bottomed flask, FE (701 mg) was dissolved in mixed solvents of ethanol and CH₂Cl₂ (4:1) (20 mL). To this solution, 10% Pd/C (72 mg, 10% w/w) was added in one portion and the resulting dark suspension was stirred under a hydrogen atmosphere for 20 h at room temperature (one hydrogen balloon). The reaction mixture was filtered through celite and rinsed with CH₂Cl₂ (3×50 mL). The filtrate was evaporated and the resulting residue proceeded to the next step without any purification.

Colorless oil; 462 mg, yield 65%; 1H NMR (400 MHz, Chloroform-d) δ 3.66 (s, 3H), 2.70-2.15 (m, 1H), 1.99-1.66 (m, 4H), 1.56-1.46 (m, 2H), 1.39-1.04 (m, 16H), 0.93-0.78 (m, 10H).

### Example 58—Preparation of Compound HME (Hydrogenation Reaction, F)

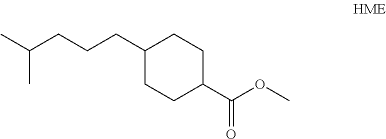 [SMILES: COC(=O)C1CCC(CCCC(C)C)CC1]

In a 100 mL round-bottomed flask, ME (1.2 g) was dissolved in mixed solvents of ethanol and CH₂Cl₂ (4:1) (20 mL). To this solution, 10% Pd/C (126 mg, 10% w/w) was added in one portion and the resulting dark suspension was stirred under a hydrogen atmosphere for 20 h at room temperature (one hydrogen balloon). The reaction mixture was filtered through celite and rinsed with CH₂Cl₂ (3×50 mL). The filtrate was evaporated and the resulting residue proceeded to the next step without any purification.

Colorless oil; 0.88 g, yield 72%; 1H NMR (400 MHz, Chloroform-d) δ 3.65 (s, 3H), 2.71-2.14 (m, 1H), 2.01-1.67 (m, 4H), 1.59-1.04 (m, 11H), 0.94-0.78 (m, 7H).

### Example 59—Preparation of Compound HFE-211 (Transesterification Reaction, G)

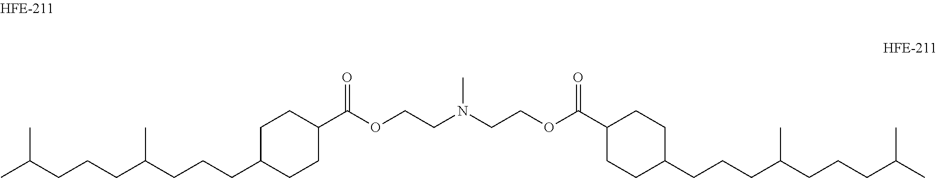 [SMILES: CC(C)CCCC(C)CCCC1CCC(C(=O)OCCN(C)CCOC(=O)C2CCC(CCCC(C)CCCC(C)C)CC2)CC1]

In a 4 mL glass tube, HFE (155 mg, 0.5 mmol) is combined with an amine 211 (25 mg, 0.2 mmol) and triazabicyclodecene (TBD) (12 mg, 0.1 mmol). The tube was sealed and the reaction mixture was stirred for 16 h at 130° C. The cooled reaction mixture was purified by silica gel flash column chromatography with 20% EtOAc/hexane.

Colorless oil; 106 mg, yield 78%; 1H NMR (400 MHz, Chloroform-d) δ 4.16-3.98 (m, 4H), 2.70-2.57 (m, 4H), 2.25 (s, 3H), 2.21-2.02 (m, 2H), 1.91-1.55 (m, 8H), 1.46-1.34 (m, 4H), 1.31-0.89 (m, 34H), 0.76-0.67 (m, 18H). 13C NMR (101 MHz, Chloroform-d) δ 176.21, 176.15, 61.72, 55.90, 55.87, 43.57, 43.51, 39.45, 37.74, 37.63, 37.43, 37.41, 37.38, 37.10, 37.04, 32.87, 32.85, 32.46, 32.37, 29.12, 28.07, 25.59, 24.90, 24.35, 24.26, 22.83, 22.73, 19.79.

### Example 60—Preparation of Compound HFE-215 (Transesterification Reaction, G)

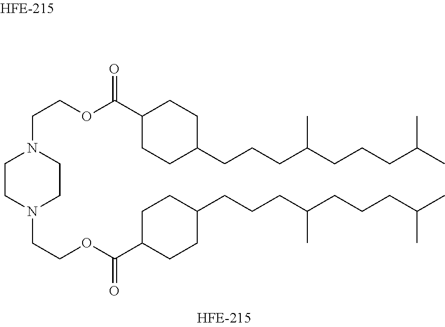 [SMILES: CC(C)CCCC(C)CCCC1CCC(C(=O)OCCN2CCN(CCOC(=O)C3CCC(CCCC(C)CCCC(C)C)CC3)CC2)CC1]

In a 4 mL glass tube, HFE (149 mg, 0.5 mmol) is combined with an amine 215 (35 mg, 0.2 mmol) and triazabicyclodecene (TBD) (12 mg, 0.1 mmol). The tube was sealed and the reaction mixture was stirred for 16 h at 130° C. The cooled reaction mixture was purified by silica gel flash column chromatography with 50% EtOAc/hexane.

Colorless oil; 92 mg, yield 65%; 1H NMR (400 MHz, Chloroform-d) δ 4.22-4.13 (m, 4H), 2.65-2.44 (m, 12H), 2.32-2.12 (m, 2H), 1.97-1.65 (m, 8H), 1.56-1.43 (m, 4H), 1.39-0.95 (m, 34H), 0.86-0.77 (m, 18H).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of Formula (I), Formula (II) or Formula (III): 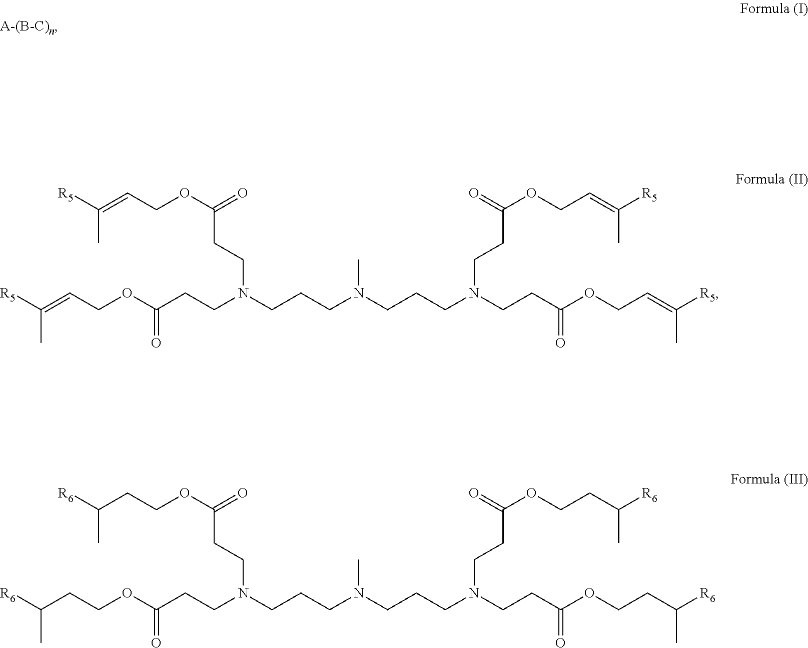
wherein:
A is A′ or A″,
A′ is: 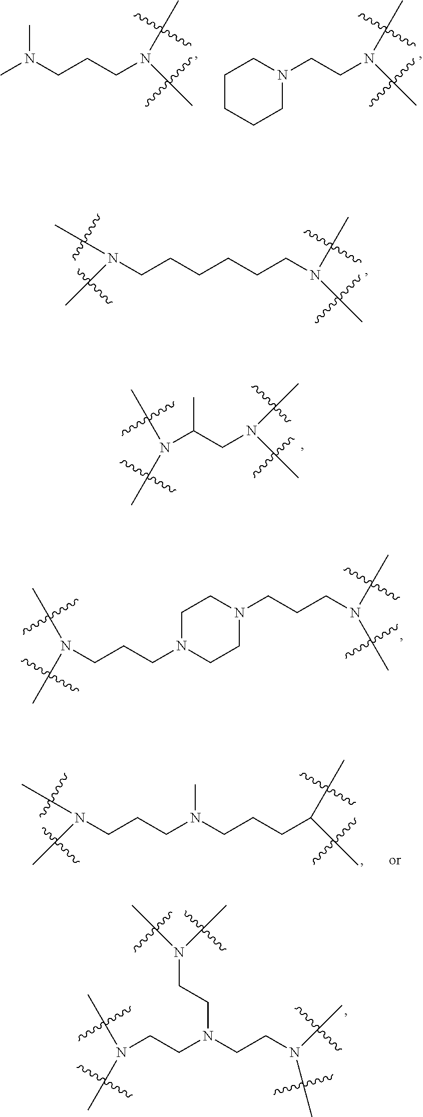
and
A″ is: 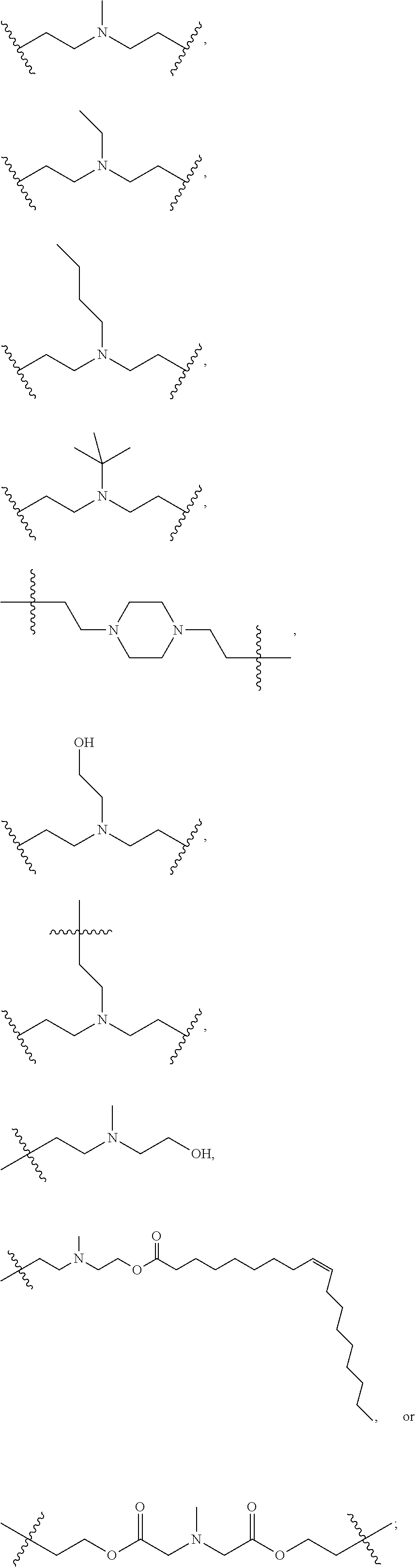
B is B′ or B″,
B′ is: 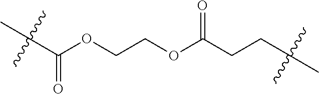 [SMILES: *CCC(=O)OCCOC(*)=O]
and
B″ is: 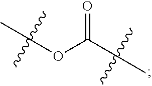 [SMILES: *OC(*)=O]
wherein when A is A′, then B is B′, and wherein when A is A″, then B is B″;
C is: 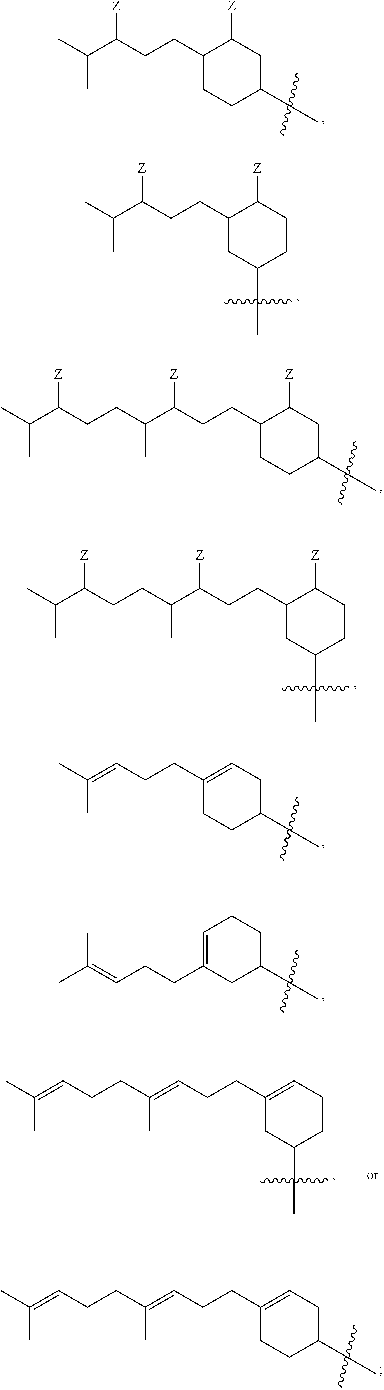
n is an integer between 1 to 6;
each Z is independently H or —S—R₁;
each R₁ is independently selected from: alkyl, 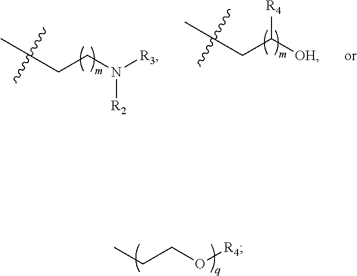
each R₂ or R₃ is independently selected from hydrogen, C₁-C₆ alkyl, C₁-C₆ alkenyl, aralkyl, or hydroxyalkyl;
each R₄ is hydrogen or C₁-C₃ alkyl;
each R₅ is 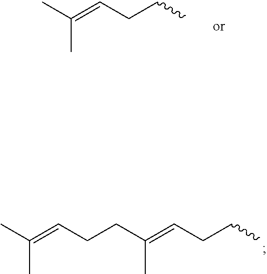
each R₆ is 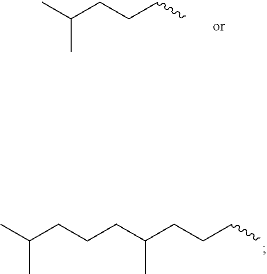
each m is an integer independently selected from 1-10; and
each q is an integer independently selected from 1-200.

2. The compound of claim 1, wherein each C is:

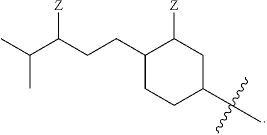

3. The compound of claim 1, wherein each C is:

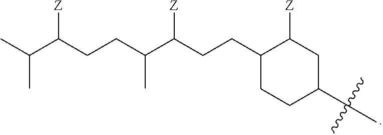

4. The compound of claim 1, wherein each C is:

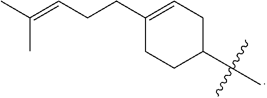 [SMILES: *C1CC=C(CCC=C(C)C)CC1]

5. The compound of claim 1, wherein each C is:

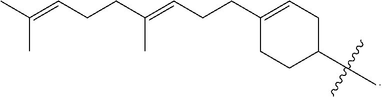 [SMILES: *C1CC=C(CC/C=C(\C)CCC=C(C)C)CC1]

6. The compound according to claim 2, wherein Z is H.

7. The compound according to claim 2, wherein Z is —S—R₁, and wherein at least one R₁ is alkyl.

8. The compound of claim 7, wherein the at least one R₁ is C₁₀H₂₁.

9. The compound of claim 7, wherein the at least one R₁ is C₂H₄(CH)(CH₃)₂.

10. The compound according to claim 1, wherein A is A′ and B is B′.

11. The compound according to claim 1, wherein A′

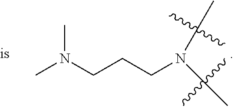 [SMILES: *N(*)CCCN(C)C]

12. The compound according to claim 1, wherein A′ is

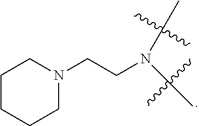 [SMILES: *N(*)CCN1CCCCC1]

13. The compound according to claim 1, wherein A′ is

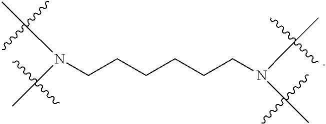 [SMILES: *N(*)CCCCCCN(*)* ; "."]

14. The compound according to claim 1, wherein A′ is

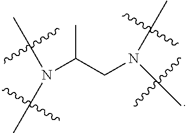 [SMILES: *N(*)CC(C)N(*)* ; "."]

15. The compound according to claim 1, wherein A′ is

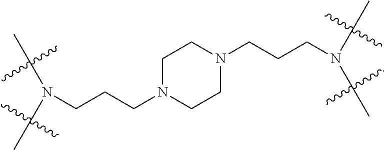 [SMILES: *N(*)CCCN1CCN(CCCN(*)*)CC1 ; "."]

16. The compound according to claim 1, wherein A′ is

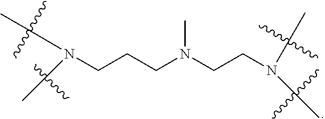 [SMILES: *N(*)CCCN(C)CCN(*)* ; "."]

17. The compound according to claim 1, wherein A′ is

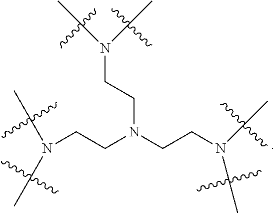 [SMILES: *N(*)CCN(CCN(*)*)CCN(*)* ; "."]

18. The compound according to claim 1, wherein A is A″ and B is B″.

19. The compound according to claim 1, wherein A″ is

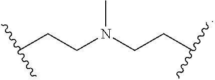 [SMILES: *CCN(C)CC* ; "."]

20. The compound according to claim 1, wherein A″ is

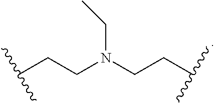 [SMILES: *CCN(CC)CC* ; "."]

21. The compound according to claim 1, wherein A″ is

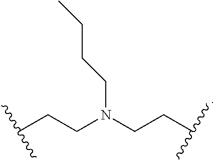 [SMILES: *CCN(CC*)CCCC ; "."]

22. The compound according to claim 1, wherein A″ is

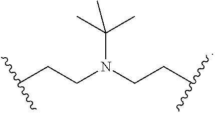 [SMILES: *CCN(CC*)C(C)(C)C ; "."]

23. The compound according to claim 1, wherein A″ is

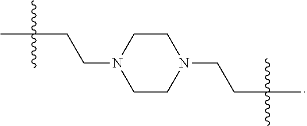 [SMILES: *CCN1CCN(CC*)CC1 ; "."]

24. The compound according to claim 1, wherein A″ is

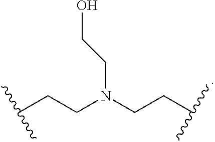 [SMILES: *CCN(CC*)CCO ; "."]

25. The compound according to claim 1, wherein A″ is

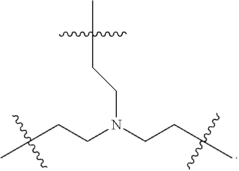 [SMILES: *CCN(CC*)CC* ; "."]

26. The compound according to claim 1, wherein A″ is

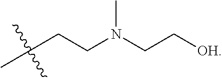 [SMILES: *CCN(C)CCO]

27. The compound according to claim 1, wherein A″ is

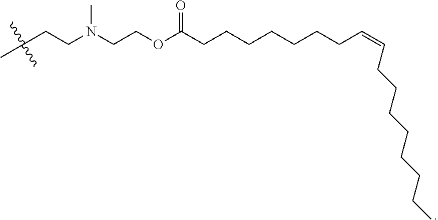 [SMILES: *CCN(C)CCOC(=O)CCCCCCC/C=C\CCCCCCCC ; "."]

28. The compound according to claim 1, wherein A″ is

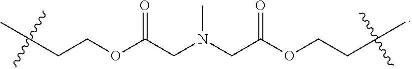 [SMILES: *CCOC(=O)CN(C)CC(=O)OCC* ; "."]

29. The compound of claim 1, wherein the compound is:

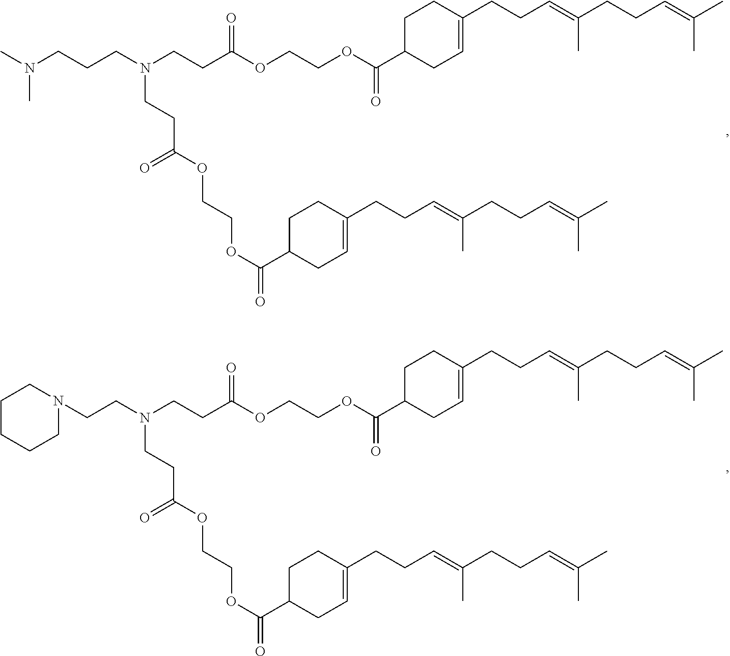 [SMILES: CC(C)=CCC/C(C)=C/CCC1=CCC(C(=O)OCCOC(=O)CCN(CCCN(C)C)CCC(=O)OCCOC(=O)C2CC=C(CC/C=C(\C)CCC=C(C)C)CC2)CC1 ; CC(C)=CCC/C(C)=C/CCC1=CCC(C(=O)OCCOC(=O)CCN(CCC(=O)OCCOC(=O)C2CC=C(CC/C=C(\C)CCC=C(C)C)CC2)CCN2CCCCC2)CC1]

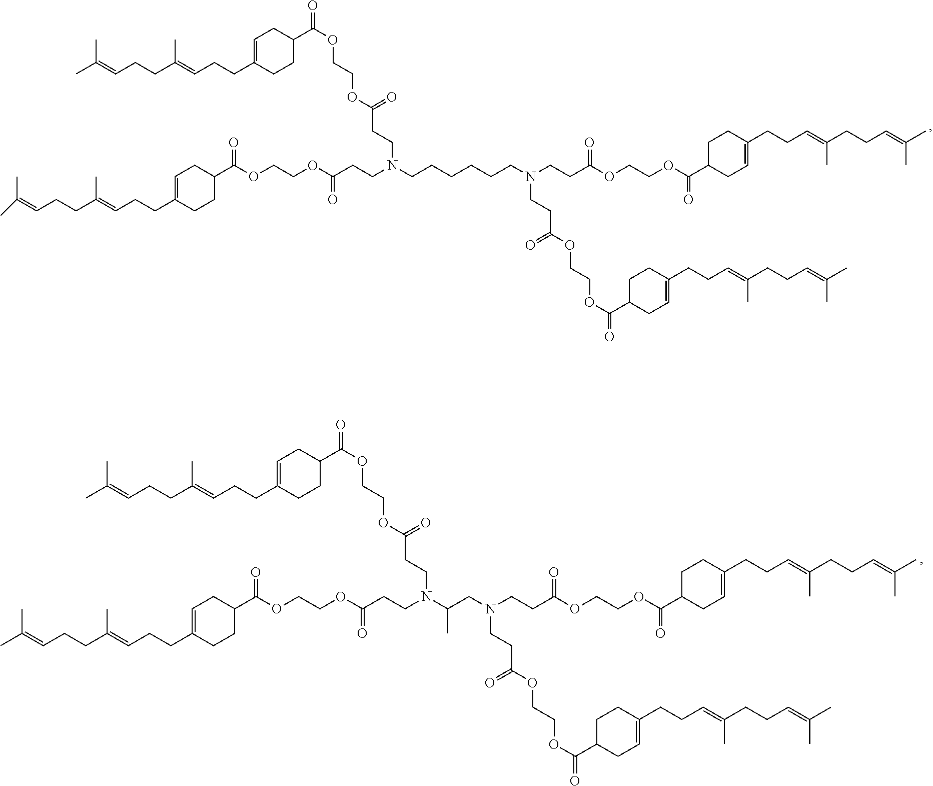

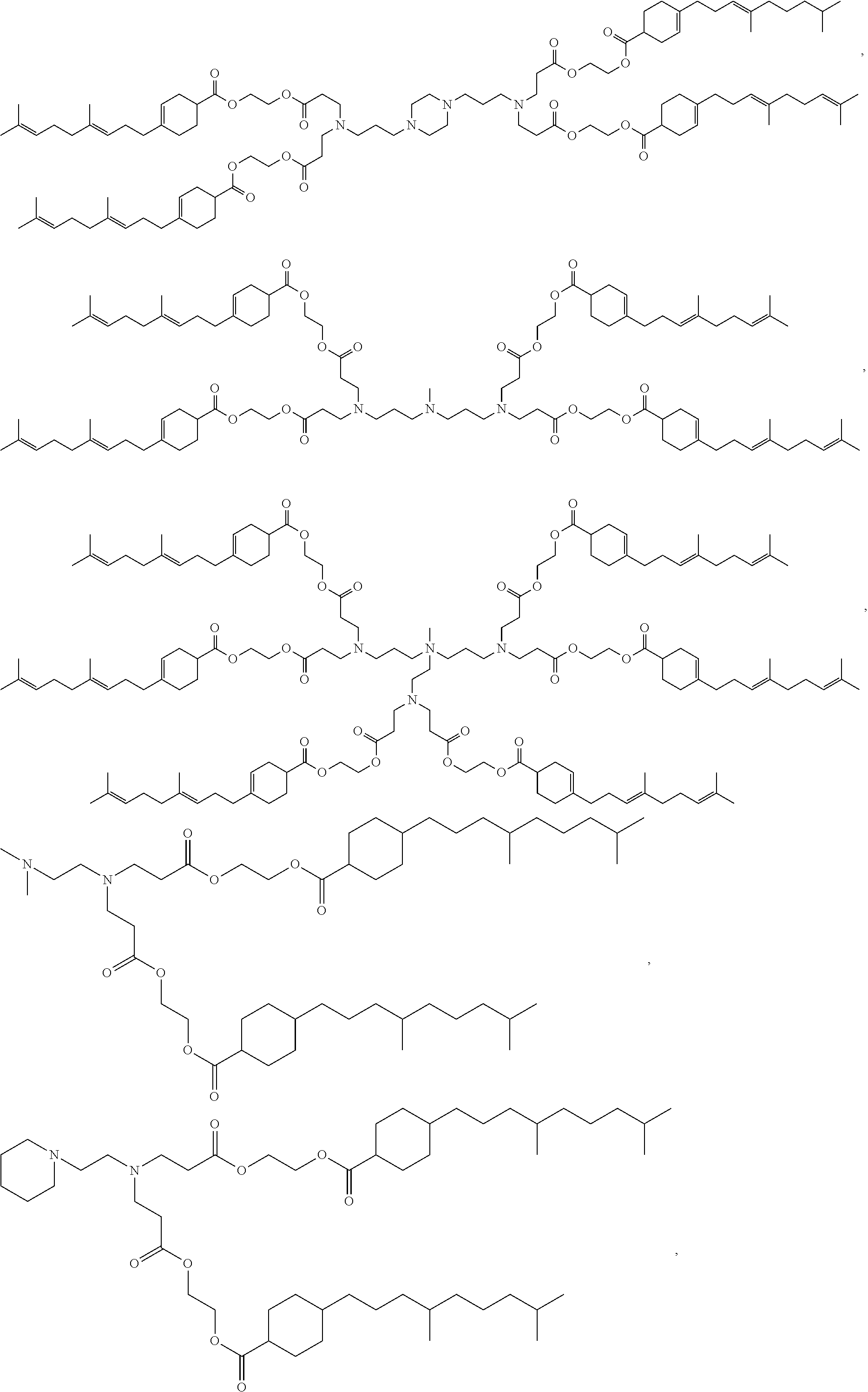

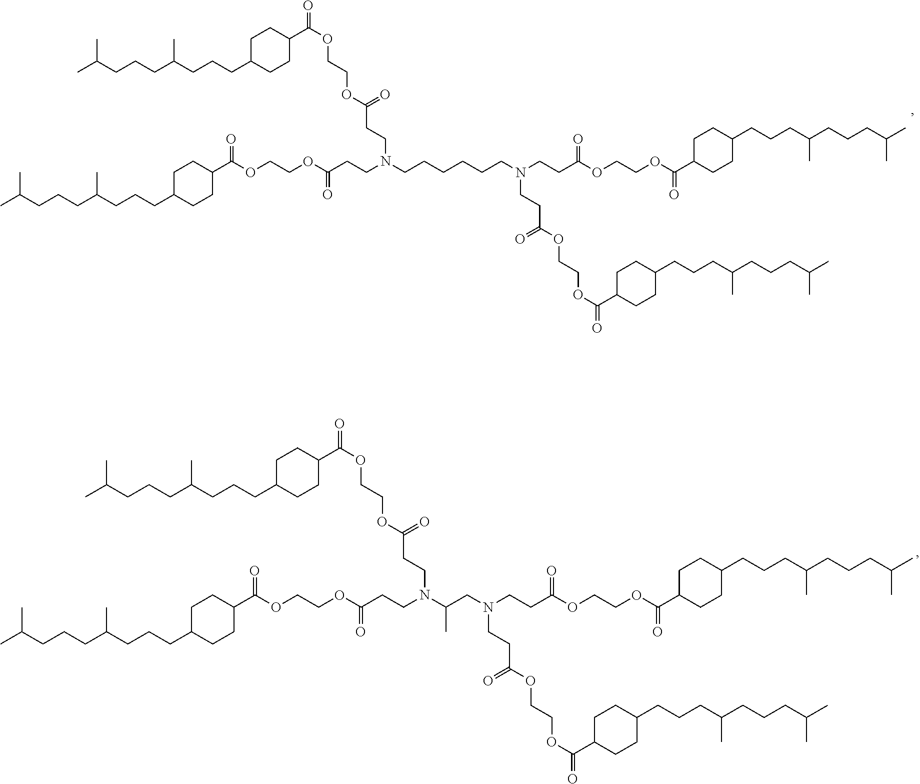

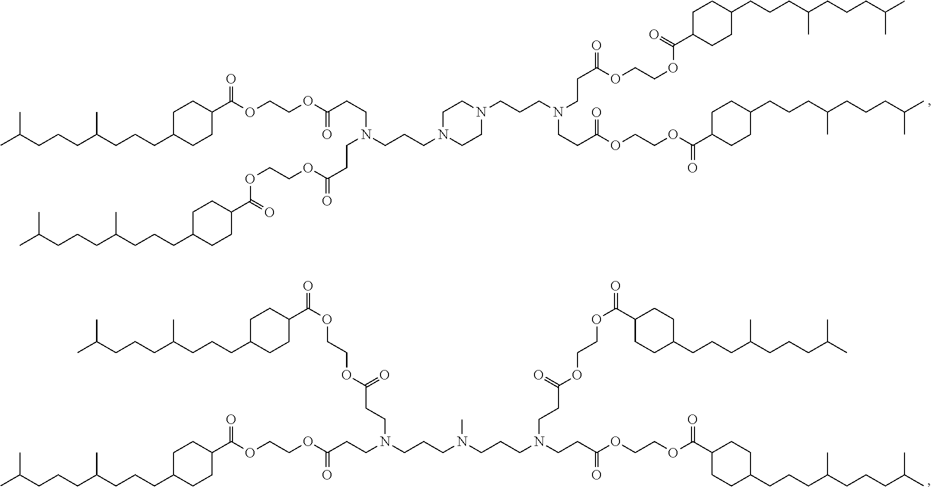

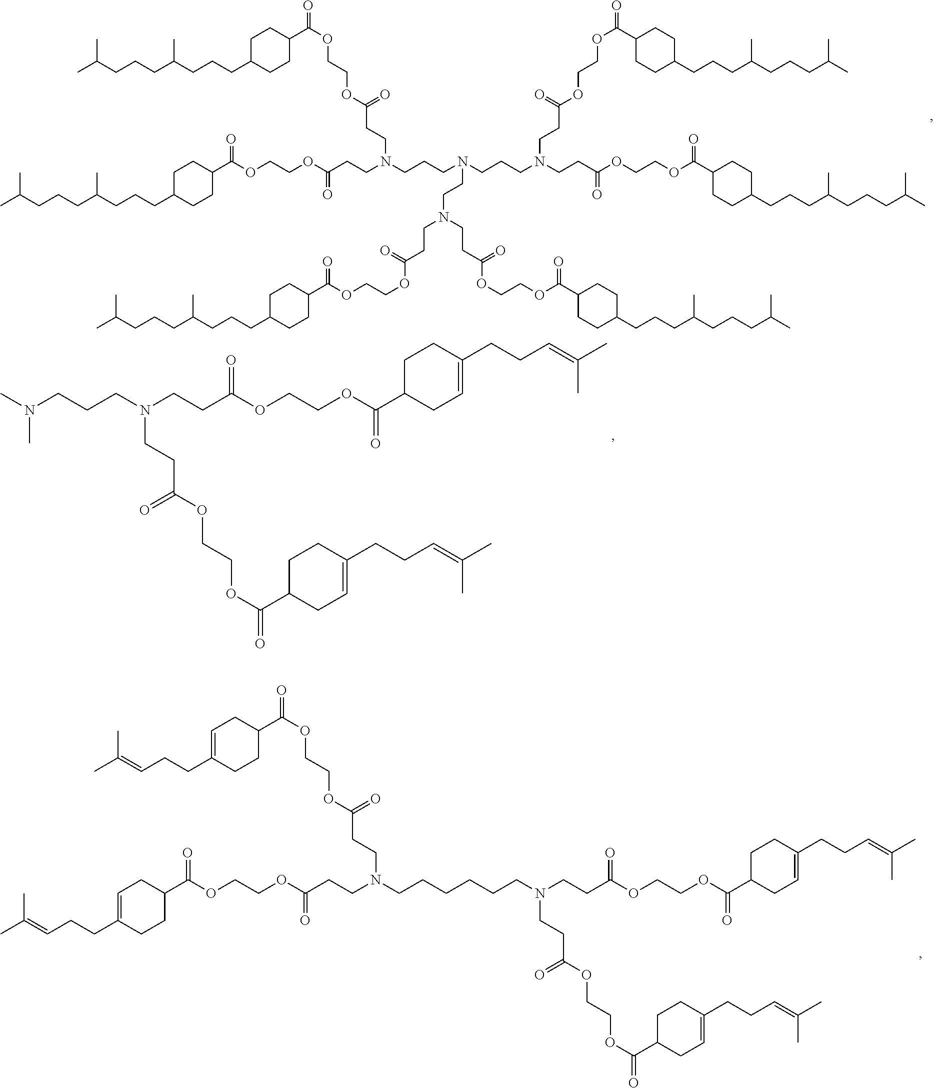

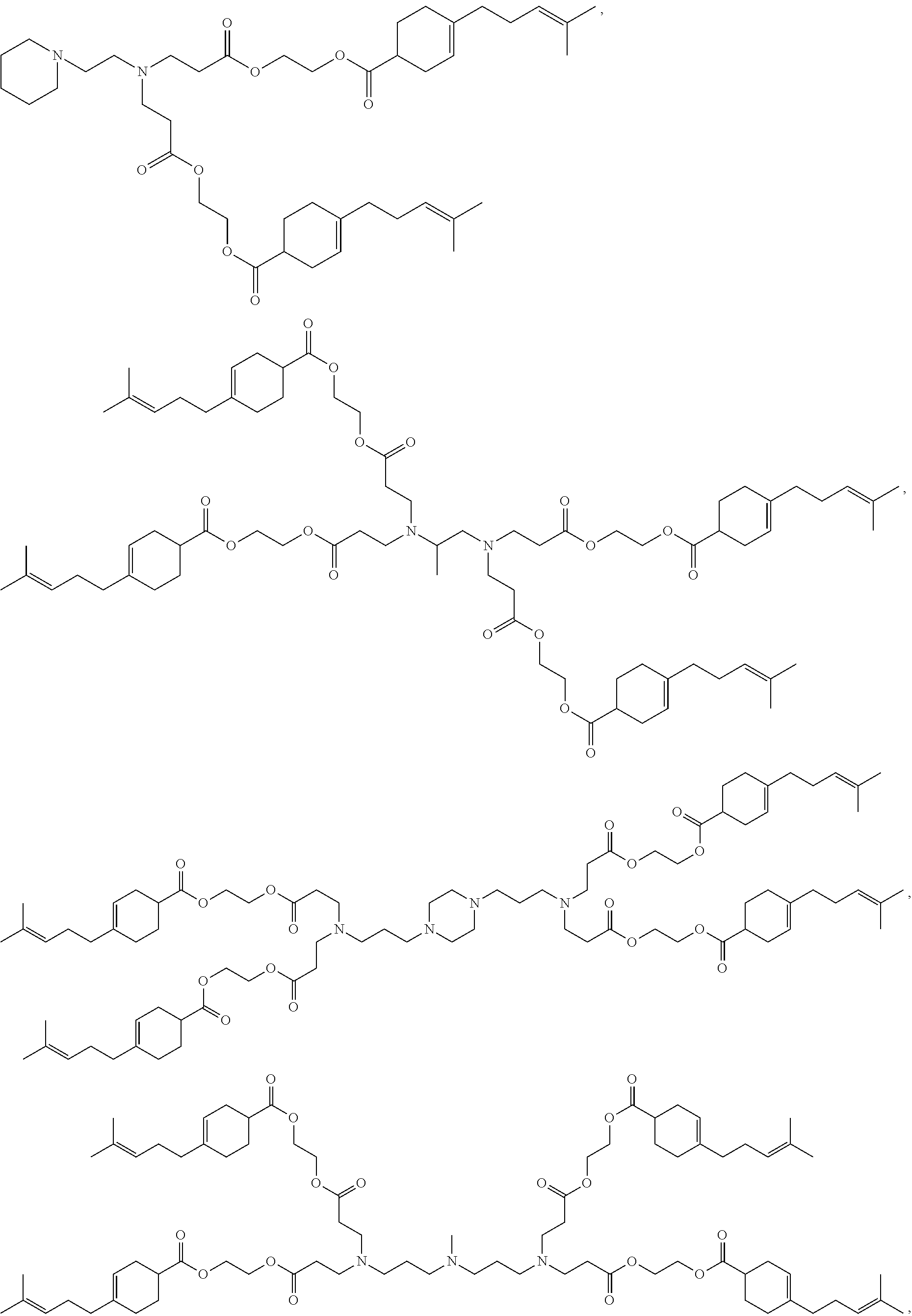

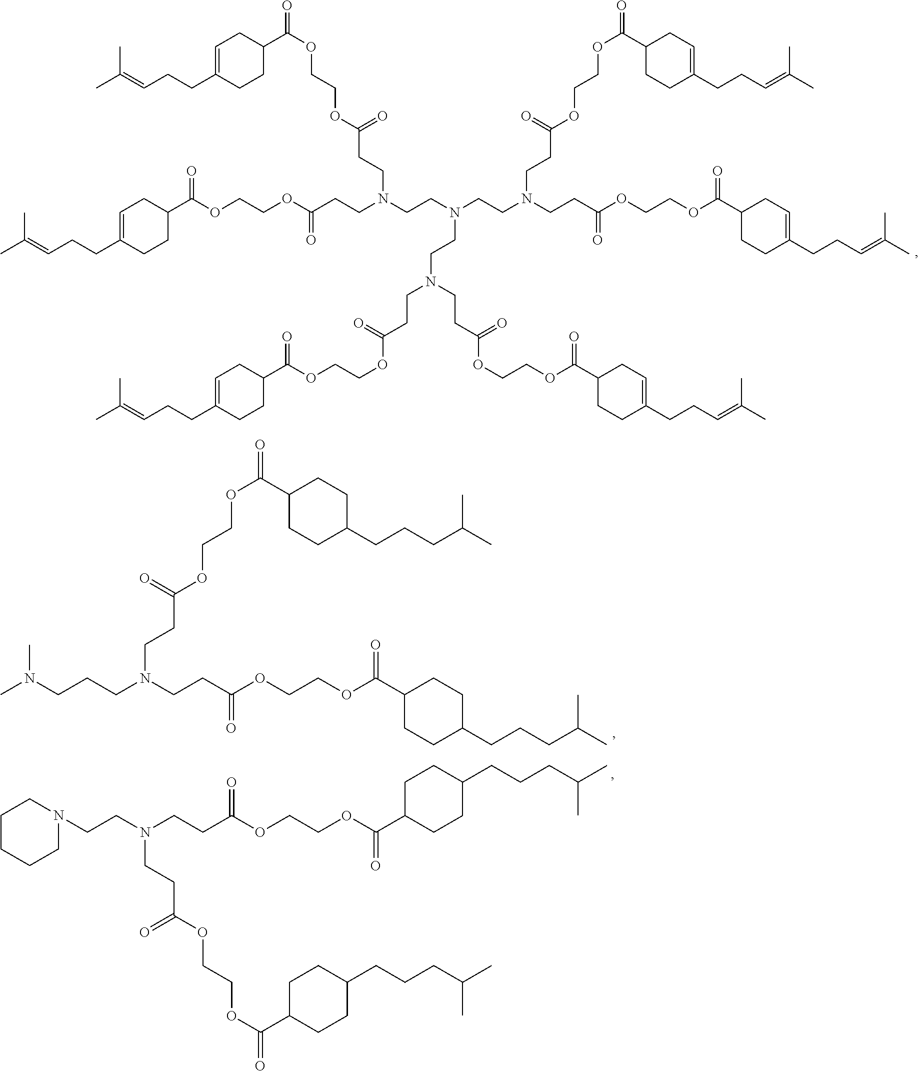

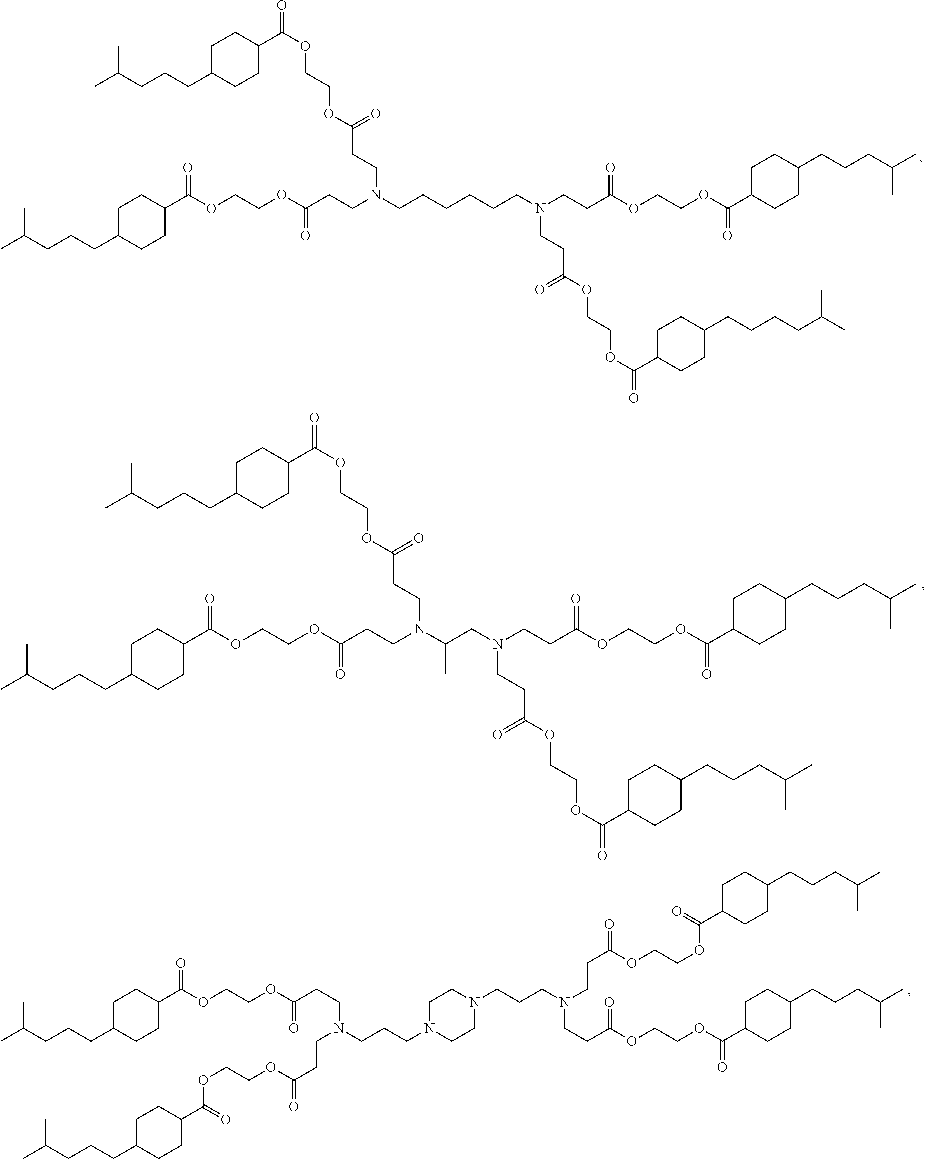

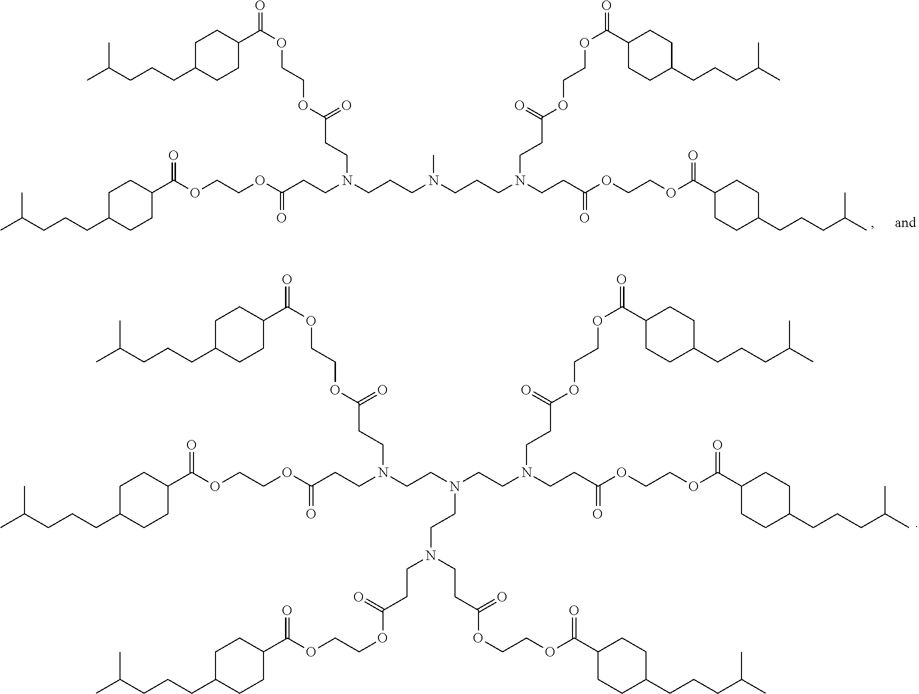

30. The compound of claim 1, wherein the compound is:

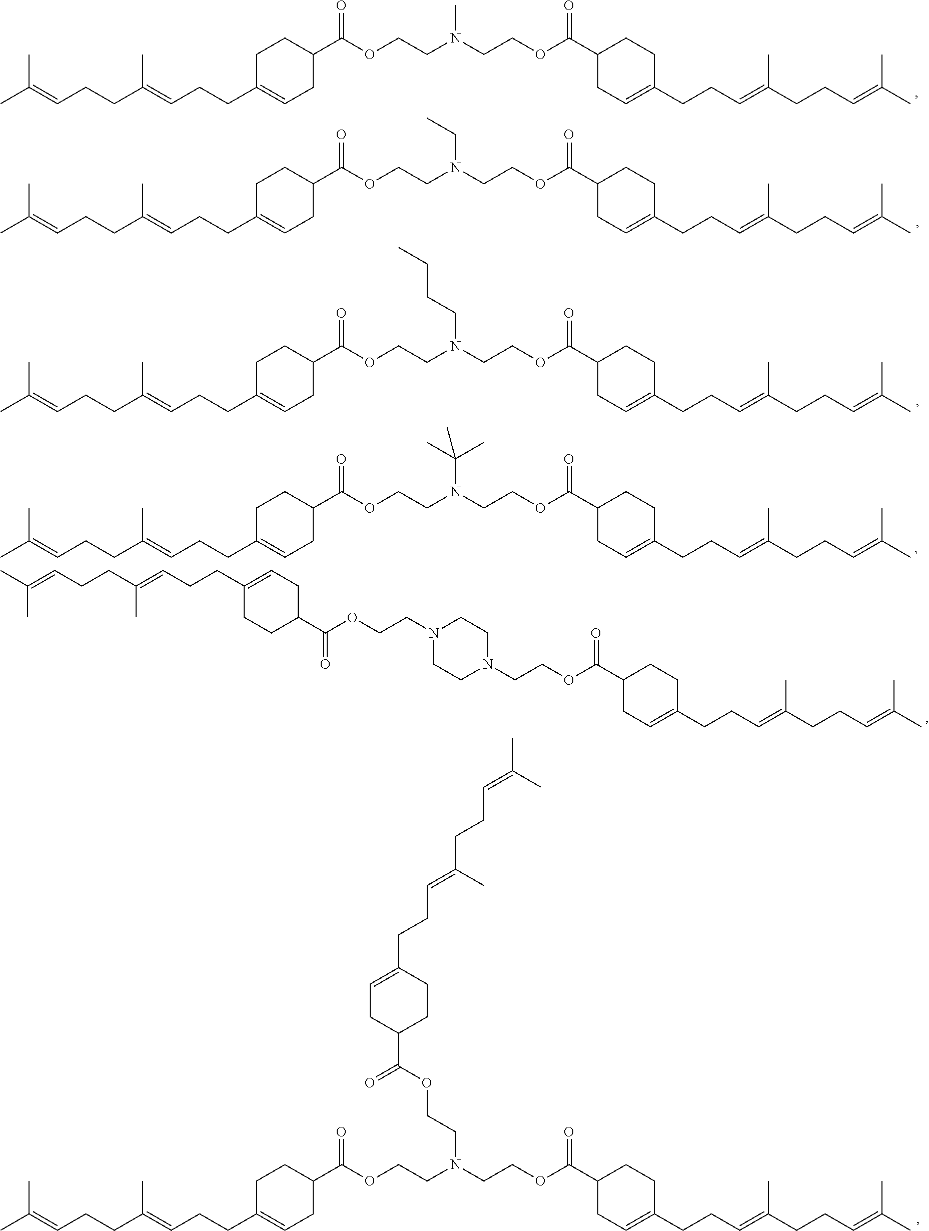

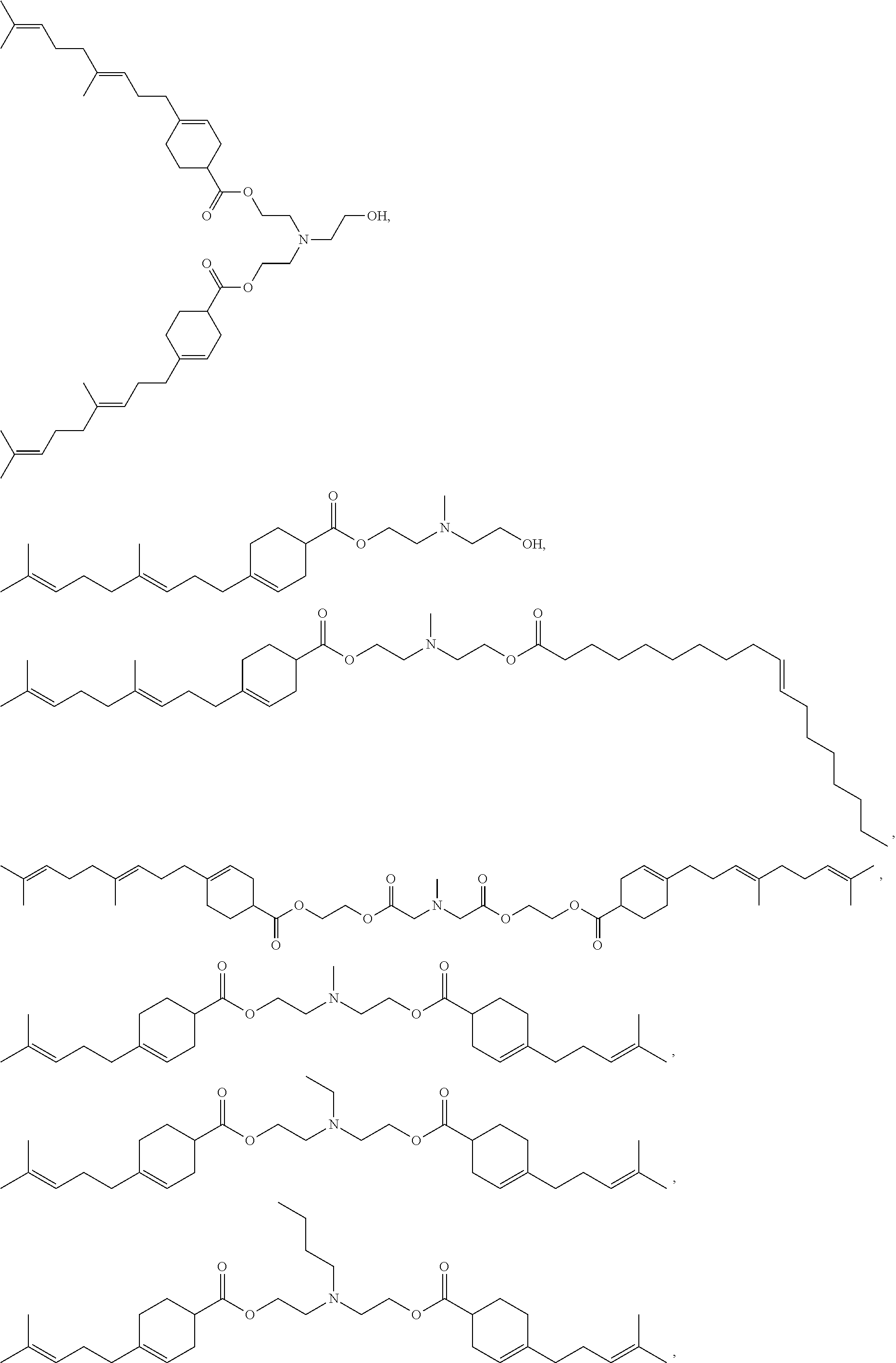

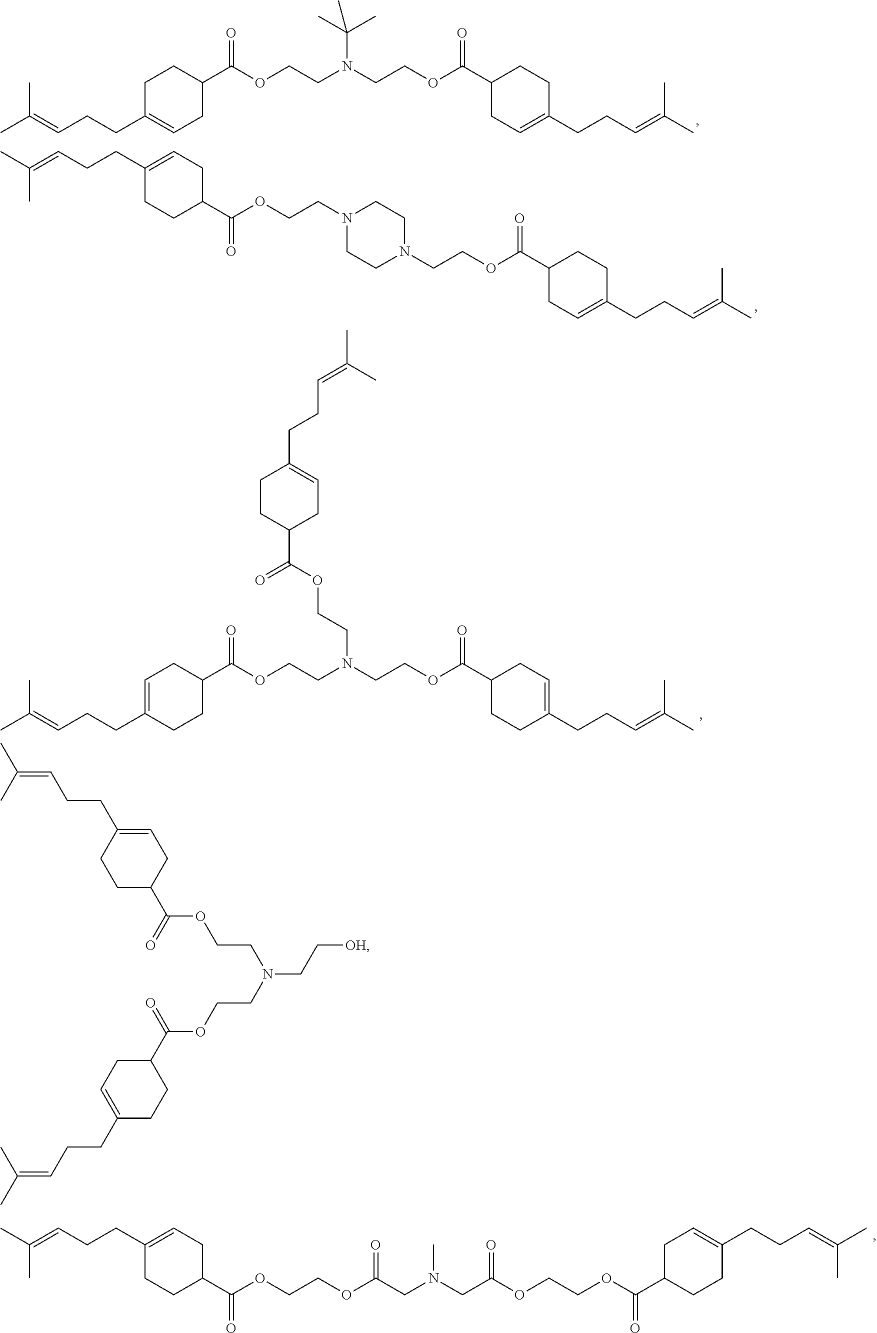

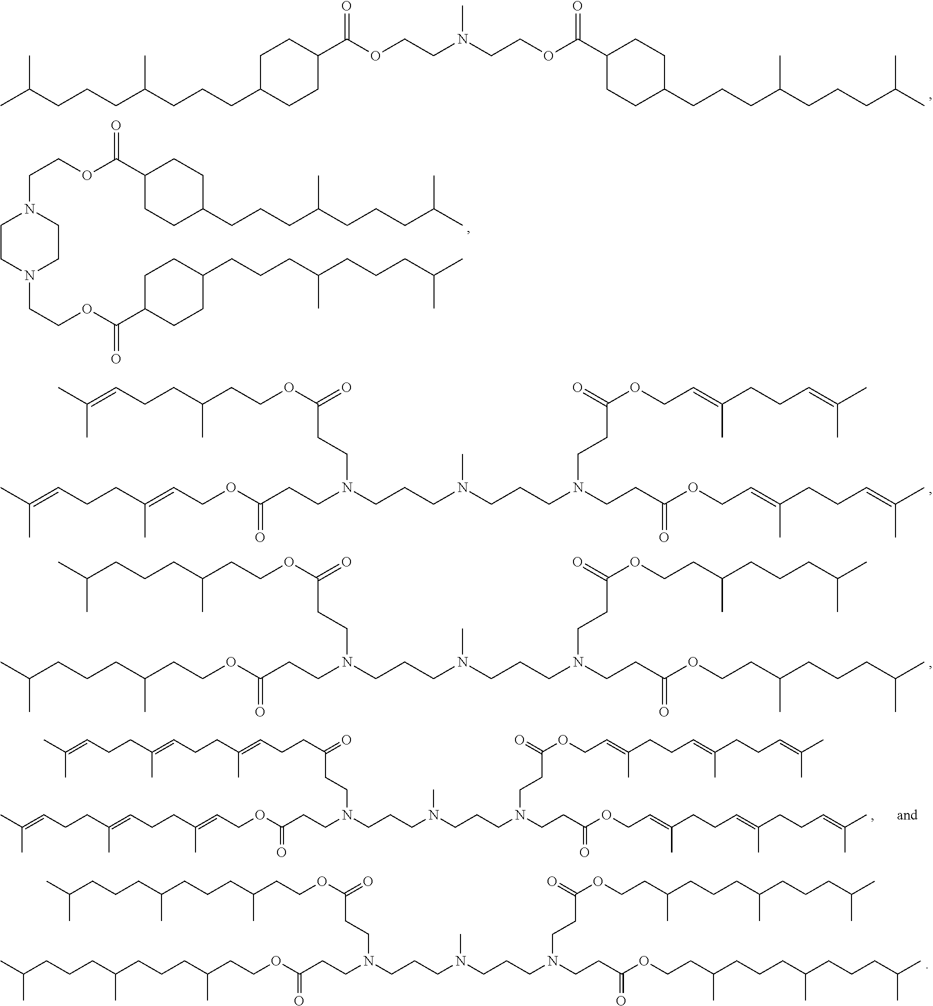

31. A pharmaceutical composition, comprising at least one nucleic acid, a compound of claim 1, and at least pharmaceutically acceptable excipient or diluent.

32. A lipoplex nanoparticle, comprising at least one nucleic acid and a compound of claim 1.

33. A method for delivering a nucleic acid to a cell, tissue, organ, animal or subject, comprising contacting the cell, the tissue, the organ, the animal or the subject with an effective amount of the pharmaceutical composition of claim 31.
